SEQUENCE LISTING (24 sequences)

SEQ ID NO: 1 - protein, 8 aa, synthetic construct
SIINFEKL

SEQ ID NO: 2 - RNA, 36 nt, synthetic construct
  source 1..36: mol_type = other RNA
  modified_base 1: mod_base = OTHER; note = AltR1 proprietary end-modification - proprietary/trademark name of IDT
  modified_base 36: mod_base = OTHER; note = AltR2 proprietary end-modification - proprietary/trademark name of IDT
ctcgagcactggaccaatacgttttagagctatgct

SEQ ID NO: 3 - DNA, 24 bp, synthetic construct
  source 1..24: mol_type = other DNA
aggatgaccaagtcaaaaagattg

SEQ ID NO: 4 - DNA, 20 bp, synthetic construct
  source 1..20: mol_type = other DNA
ggaatggaccggacaaaggt

SEQ ID NO: 5 - DNA, 21 bp, synthetic construct
  source 1..21: mol_type = other DNA
gtttctctatttggccccgac

SEQ ID NO: 6 - DNA, 23 bp, synthetic construct
  source 1..23: mol_type = other DNA
agagatactggtcaaccttctgc

SEQ ID NO: 7 - DNA, 21 bp, synthetic construct
  source 1..21: mol_type = other DNA
aactcttgccacacaaggtct

SEQ ID NO: 8 - DNA, 20 bp, synthetic construct
  source 1..20: mol_type = other DNA
tcccacatgatgccctgaac

SEQ ID NO: 9 - DNA, 22 bp, synthetic construct
  source 1..22: mol_type = other DNA
atggttcctgctactgtctcca

SEQ ID NO: 10 - DNA, 21 bp, synthetic construct
  source 1..21: mol_type = other DNA
ttaatgcggtcgcacgtgtct

SEQ ID NO: 11 - DNA, 21 bp, synthetic construct
  source 1..21: mol_type = other DNA
cttagtcctttcgcctaccca

SEQ ID NO: 12 - DNA, 19 bp, synthetic construct
  source 1..19: mol_type = other DNA
gcgggtcatgttgaagctg

SEQ ID NO: 13 - DNA, 23 bp, synthetic construct
  source 1..23: mol_type = other DNA
acccggatcatttatggaggttc

SEQ ID NO: 14 - DNA, 20 bp, synthetic construct
  source 1..20: mol_type = other DNA
ggcagtgctcattgtttggc

SEQ ID NO: 15 - DNA, 20 bp, synthetic construct
  source 1..20: mol_type = other DNA
accacagtccatgccatcac

SEQ ID NO: 16 - DNA, 20 bp, synthetic construct
  source 1..20: mol_type = other DNA
tccaccaccctgttgctgta

SEQ ID NO: 17 - DNA, 20 bp, synthetic construct
  source 1..20: mol_type = other DNA
gcactgcccttctggaatga

SEQ ID NO: 18 - DNA, 21 bp, synthetic construct
  source 1..21: mol_type = other DNA
cctcttctgagagaccctcca

SEQ ID NO: 19 - DNA, 19 bp, synthetic construct
  source 1..19: mol_type = other DNA
gcagcgactcgtcttcact

SEQ ID NO: 20 - DNA, 21 bp, synthetic construct
  source 1..21: mol_type = other DNA
gcatggttcctgaagtcctcg

SEQ ID NO: 21 - DNA, 20 bp, synthetic construct
  source 1..20: mol_type = other DNA
ggagtggtgtgaatgttgcc

SEQ ID NO: 22 - DNA, 21 bp, synthetic construct
  source 1..21: mol_type = other DNA
gatcacctcgtaggcactgtc

SEQ ID NO: 23 - DNA, 21 bp, synthetic construct
  source 1..21: mol_type = other DNA
gatcaagatcattgctcctga

SEQ ID NO: 24 - DNA, 20 bp, synthetic construct
  source 1..20: mol_type = other DNA
cagctcagtaacagtccgcc

(19) United States
(12) Patent Application Publication
(10) Pub. No.: US 2024/0358705 A1
(43) Pub. Date: Oct. 31, 2024

(54) COMPOSITIONS AND METHODS FOR TREATMENT OF CANCER

(71) Applicant: Board of Supervisors of Louisiana State University and Agricultural and Mechanical College, Baton Rouge, LA (US)

(72) Inventors: Phaethon Philbrook, New Orleans, LA (US); Augusto Ochoa, New Orleans, LA (US); Matthew Dean, New Orleans, LA (US)

(21) Appl. No.: 18/649,074

(22) Filed: Apr. 29, 2024

Related U.S. Application Data
(63) Continuation-in-part of application No. PCT/US2022/048410, filed on Oct. 31, 2022.
(60) Provisional application No. 63/273,691, filed on Oct. 29, 2021.
(60) Provisional application No. 63/273,698, filed on Oct. 29, 2021.
(60) Provisional application No. 63/337,230, filed on May 2, 2022.
(60) Provisional application No. 63/346,098, filed on May 26, 2022.

(51) Int. Cl. A61K 31/519; A61K 31/136; A61K 31/192; A61K 31/198; A61K 31/282; A61K 31/337; A61K 31/513; A61K 31/675; A61K 31/704; A61K 31/7068; A61K 39/00; A61K 39/395; A61K 45/06; A61P 35/00; C12N 5/0783

(52) U.S. Cl. CPC A61K 31/519; A61K 31/136; A61K 31/192; A61K 31/198; A61K 31/282; A61K 31/337; A61K 31/513; A61K 31/675; A61K 31/704; A61K 31/7068; A61K 39/3955; A61K 39/4611; A61K 39/4613; A61K 39/4631; A61K 39/4632; A61K 45/06; A61P 35/00; C12N 5/0636; C12N 2501/51; C12N 2501/515

(57) ABSTRACT

The invention provides a method of preventing, treating, and/or preventing the recurrence of cancer in a subject by administering to the subject a therapeutically effective amount of a GPR84 antagonist. Further, the invention provides a method of modulating the immune system of a subject afflicted with or at risk of cancer. Still further, the invention provides a method of preventing or treating an inflammatory response in a subject. Also provided is a method of preventing an immune system disease or disorder in a subject. Further, the invention provides a method treating an immune system disease or disorder in a subject. Also, the invention provides a method of modulating the immune system of a subject afflicted with or at risk of an immune system disease or disorder.

This application is a Continuation-in-Part of International Application PCT/US2022/048410, filed on Oct. 31, 2022, which claims priority from U.S. Provisional Patent Application No. 63/273,691, filed on Oct. 29, 2021; U.S. Provisional Patent Application No. 63/273,698, filed on Oct. 29, 2021; U.S. Provisional Patent Application No. 63/337,230, filed on May 2, 2022; and U.S. Provisional Application No. 63/346,098, filed on May 26, 2022, the contents of each of which are incorporated herein by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. P20 CA233374 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Jul. 11, 2024, is named 2932719-000182-US4_SL.xml and is 23,409 bytes in size.

### FIELD OF THE INVENTION

This invention is directed to compositions and methods for treating or preventing GPR84 responsive conditions. For example, the invention provides a method of preventing or treating cancer in a subject by administering to the subject a therapeutically effective amount of a GPR84 antagonist. Also provided is a method of preventing recurrence of cancer in a subject by administering to the subject a therapeutically effective amount of a GPR84 antagonist. Further, the invention provides a method of modulating the immune system of a subject afflicted with or at risk of cancer by administering to the subject a therapeutically effective amount of a GPR84 antagonist. The invention provides a method of preventing or treating an inflammatory response in a subject by administering to the subject a therapeutically effective amount of a GPR84 agonist. Also provided is a method of preventing an immune system disease or disorder in a subject by administering to the subject a therapeutically effective amount of a GPR84 agonist. Further, the invention provides a method treating an immune system disease or disorder in a subject by administering to the subject a therapeutically effective amount of a GPR84 agonist. Also, the invention provides a method of modulating the immune system of a subject afflicted with or at risk of an immune system disease or disorder by administering to the subject a therapeutically effective amount of a GPR84 agonist.

### BACKGROUND OF THE INVENTION

GPR84 is a medium-chain fatty acid receptor. It was found through a data mining strategy searching for GPCRs. Still defined as an orphan receptor, its ligand, MCFAs, was not discovered until years later. Further examination found that GPR84 is Gi/o GPCR, and interaction with its ligand leads to an intracellular reduction of cyclic adenosine monophosphate (cAMP). GPR84 is distributed among various tissues in the body, such as adipocytes, but also, GPR84 is found to have expression in immune cells. In studies of adipogenesis, GPR84 was upregulated in human adipocytes stimulated with inflammatory cytokines and mice fed with high-fat diet. In the immune tissue, GPR84 is expressed in monocytes, macrophages, granulocytes, T cells, and B cells.

### SUMMARY OF THE INVENTION

Aspects of the invention are drawn to a method of preventing cancer in a subject. In embodiments, the method comprises administering to the subject a therapeutically effective amount of a GPR84 antagonist.

Aspects of the invention are also drawn towards a method of preventing recurrence of cancer in a subject. In embodiments, the method comprises administering to the subject a therapeutically effective amount of a GPR84 antagonist.

Still further, aspects of the invention are drawn towards a method treating cancer in a subject. In embodiments, the method comprising administering to the subject a therapeutically effective amount of a GPR84 antagonist.

Also, aspects of the invention are drawn towards a method of modulating the immune system of a subject afflicted with or at risk of cancer. In embodiments, the method comprises administering to the subject a therapeutically effective amount of a GPR84 antagonist.

In embodiments, modulating comprises increasing the cytotoxicity of T-cells, decreasing immunosuppressive effects of myeloid-derived-suppressor cells, or both.

In embodiments, the GPR84 antagonist comprises a compound according to:

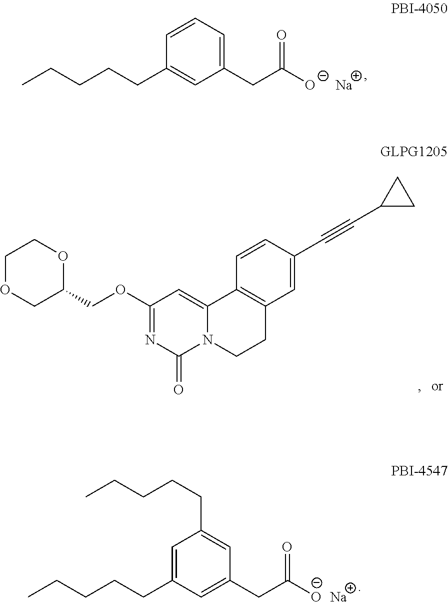 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

In embodiments, the therapeutically effective amount comprises about 10 mg/kg to about 90 mg/kg.

In embodiments, the GPR84 antagonist is administered daily, weekly, biweekly, or monthly.

In embodiments, the method further comprises administering to the subject at least one additional anti-cancer agent. For example, the at least one additional anti-cancer agent comprises an immunotherapeutic agent, a chemotherapeutic agent, radiation therapy, or any combination thereof. For example, the immunotherapeutic agent comprises an immune checkpoint inhibitor, an adoptive T-cell therapy, a monoclonal antibody, an oncolytic virus therapy, a cancer vaccine, an immune system modulator, or any combination thereof. For example, the monoclonal antibody comprises an immune checkpoint inhibitor. For example, the monoclonal antibody comprises anti-OX-40, anti-PD-1, anti-PD-L1, anti-LAG3, or anti-CTLA-4. For example, the anti-PD-1 antibody comprises a pembrolizumab, nivolumab, or cemiplimab. For example, the anti-CTLA-4 antibody comprises Ipilimumab. For example, the adoptive T-cell therapy comprises tumor-infiltrating lymphocyte (TIL) therapy, engineered T-cell receptor (TCR) therapy, CAR T-cell therapy, or natural killer (NK) cell therapy. For example, the at least one chemotherapeutic agent comprises cyclophosphamide, gemcitabine, 5-fluorouracil, docetaxel, doxorubicin, oxaliplatin, mitoxantrone, melphalan, or anthracyclines.

In embodiments, the cancer comprises breast cancer, prostate cancer, colorectal cancer, cervical cancer, lung cancer, lymphoma, leukemia, pancreatic cancer, liver cancer, brain cancer, or skin cancer.

In embodiments, the subject has a familial history of cancer, a chronic inflammatory condition, or a genomic mutation. For example, the inflammatory condition comprises colitis, chronic prostatitis. For example, the genomic mutation comprises a mutation of BRCA1, BRCA2, MLH1, MSH2, MSH6, PMS2, EPCAM, APC, or MUTYH.

Aspects of the invention are further drawn to a method of activating a T cell or NK cell. For example, the method comprising contacting a T cell or NK cell with a GPR84 antagonist, wherein the GPR84 antagonist activates the T cell or NK cell.

Embodiments further comprise the step of activating the T cell or NK cell with an additional stimuli. For example, the stimuli comprise anti-CD3, anti-CD28, PSA, Muc-1, MAGE, carcinoembryonic antigen (CEA) or a combination thereof.

In embodiments, the method is an ex vivo method.

In embodiments, the cell comprises a genetically engineered T cell or genetically engineered NK cell. For example, the cell comprises a CAR T cell or a CAR NK cell.

Embodiments further comprise the step of obtaining a T cell or NK cell from a subject and culturing the T cell or NK cell in a medium comprising a GPR84 antagonist.

Embodiments further comprise the step of administering to a subject a therapeutically effective amount of the GPR84 antagonist.

In embodiments, the T cell or NK cell is dormant prior to culturing.

Embodiments further comprise administering the activated T cell or activated NK cell to a subject.

Aspects of the invention are also drawn towards a pharmaceutical composition comprising a GPR84 antagonist, at least one additional anti-cancer agent, and a pharmaceutically acceptable carrier, excipient, or diluent. In embodiments, the GPR84 antagonist comprises a compound according to:

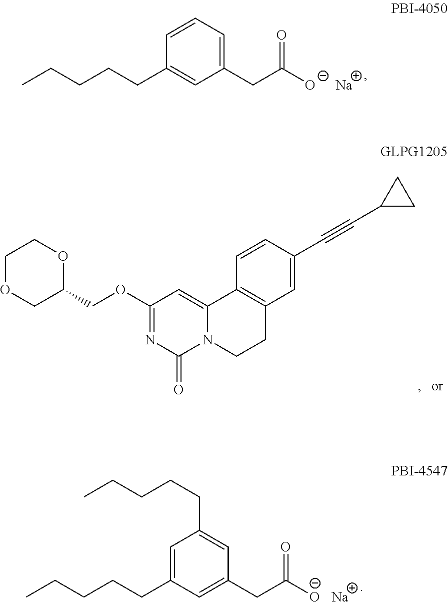 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

In embodiments, the antagonist comprises a compound as described in U.S. Pat. No. 11,098,071.

In embodiments, the at least one additional anti-cancer agent comprises an immunotherapeutic agent or a chemotherapeutic agent. For example, the immunotherapeutic agent comprises a CAR T-cell, an anti-cancer antibody, or both. For example, the anti-cancer antibody comprises an anti-checkpoint inhibitor antibody. For example, the at least one chemotherapeutic agent comprises cyclophosphamide, gemcitabine, 5-fluorouracil, docetaxel, doxorubicin, oxaliplatin, mitoxantrone, melphalan, or anthracyclines.

Aspects of the invention are drawn to a method of preventing an inflammatory response in a subject.

Further, aspects of the invention are drawn to a method of preventing an immune system disease or disorder in a subject.

Still further, aspects of the invention are drawn to a method of treating or ameliorating a symptom of an immune system disease or disorder in a subject.

Also, aspects of the invention are drawn to a method of modulating the immune system of a subject afflicted with or at risk of an immune system disease or disorder. In embodiments, modulating comprises increasing immunosuppressive effects of myeloid-derived-suppressor cells, decreasing the pro-inflammatory and cytotoxicity of T-cells, or both.

In embodiments, the GPR84 agonist comprises a compound according to:

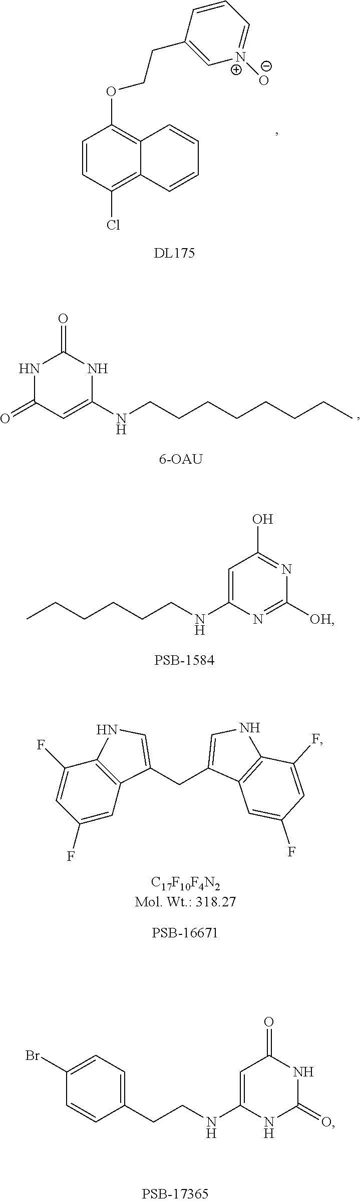

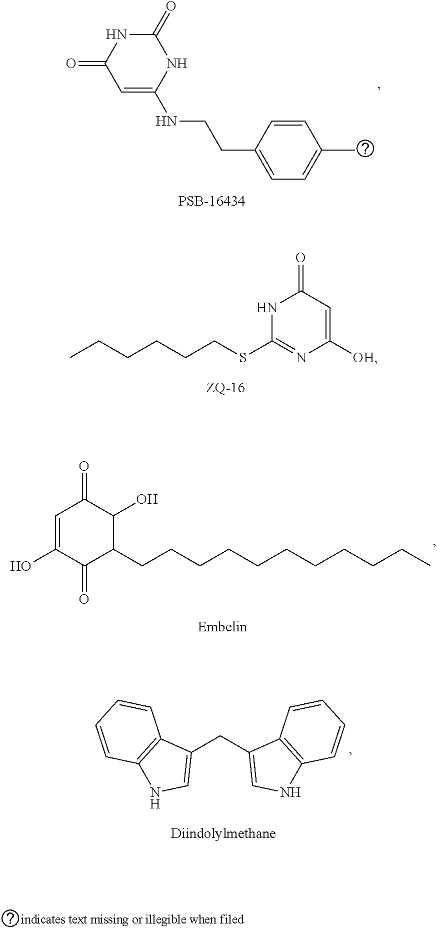

2-HTP, or medium-chain fatty acids

In embodiments, the therapeutically effective amount comprises about 10 mg/kg to about 90 mg/kg.

In embodiments, the GPR84 agonist is administered daily, weekly, biweekly, or monthly.

In embodiments, the method further comprises administering to the subject at least one additional immunomodulatory or anti-inflammatory agent. For example, the at least one additional anti-inflammatory agent or immunomodulatory treatment comprises an antibody, a corticosteroid (e.g., prednisone, prednisolone, methylprednisolone, dexamethasone, or triamcinolone), a non-steroidal anti-inflammatory, or any combination thereof. For example, the immunomodulatory treatment comprises methotrexate, a non-steroidal anti-inflammatory, or sulfasalazine. For example, the antibody comprises an anti-TNF antibody (e.g., etanercept, infliximab, adalimumab, golimumab, or certolizumab), an anti-IL-5 antibody, an anti-IL4/IL5/IL13 antibody (e.g., dupilumab), an anti-1b antibody, an anti-IL-6 antibody, an anti-IL-17 antibody, or an anti-IL-12/IL-23 antibody (e.g., ustekinumab).

In embodiments, the inflammatory response comprises inflammation.

In embodiments, the disease or disorder comprises psoriasis, psoriatic arthritis, an auto-immune disease, colitis, asthma, glomerulonephritis, hepatitis, myocarditis, transplant rejection, pre-diabetes, rheumatoid arthritis, lupus, Crohn's disease, graph versus host disease, or a combination thereof.

Aspects of the invention are also drawn to a method of culturing a population of T cells in the presence of a GPR84 agonist. For example, the T cell comprises a CD4 T cell, a CD8 T cell, or a combination thereof. In embodiments, the GPR84 agonist maintains the sternness of the population of T cells. In embodiments, the GPR84 agonist prevents the activation of the population of T cells. In embodiments, the GPR84 agonist comprises a compound according to:

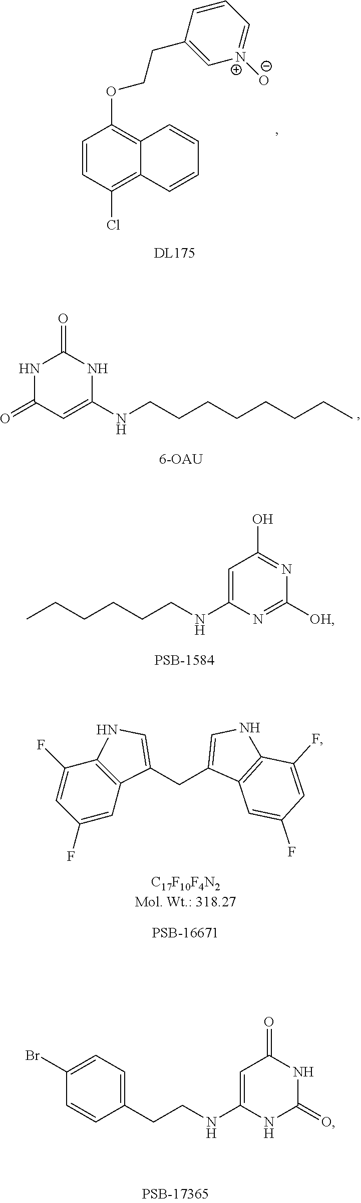

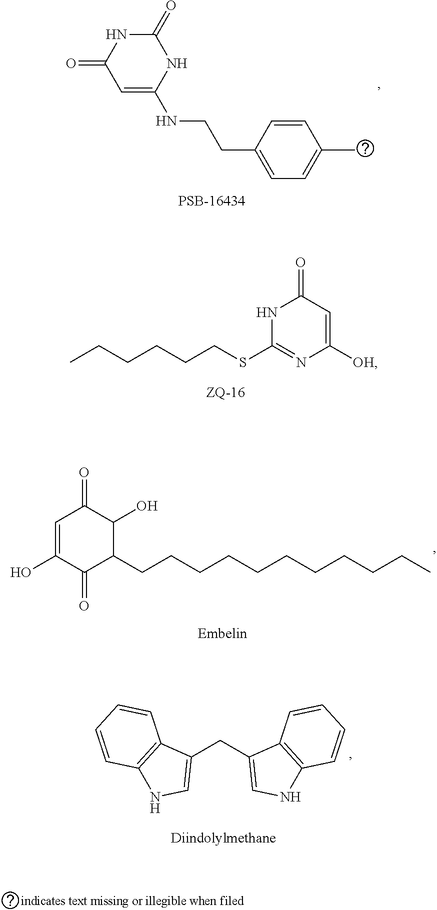

2-HTP, or medium-chain fatty acids

In embodiments, the population of T cells and/or NK cells comprises a population of CAR T cells and/or CAR NK cells.

Embodiments further comprise a step of obtaining a T cell or population of T cells from a subject.

Embodiments further comprise a step of administering the cultured population of T cells to a subject.

Aspects of the invention are also drawn towards a pharmaceutical composition comprising a GPR84 agonist, at least one additional anti-inflammatory or immunomodulatory agent, and a pharmaceutically acceptable carrier, excipient, or diluent. In embodiments, the at least one anti-inflammatory agent or at least one immunomodulatory agent comprises an antibody, a corticosteroid (e.g., prednisone, prednisolone, methylprednisolone, dexamethasone, or triamcinolone), a non-steroidal anti-inflammatory agent, or any combination thereof. For example, the immunomodulatory treatment comprises methotrexate, a non-steroidal anti-inflammatory, or sulfasalazine.

In embodiments, the antibody comprises an anti-TNF antibody (e.g., etanercept, infliximab, adalimumab, golimumab, or certolizumab), an anti-IL-5 antibody, an anti-IL4/IL5/IL13 antibody (e.g., dupilumab), an anti-1b antibody, an anti-IL-6 antibody, an anti-IL-17 antibody, or an anti-IL-12/IL-23 antibody (e.g., ustekinumab).

Other objects and advantages of this invention will become readily apparent from the ensuing description.

### BRIEF DESCRIPTION OF THE FIGURES

FIG. 1 shows a schematic describing the relationship between GPR84 and inflammation. Both chronic and acute inflammation leads to an upregulation of GPR84 on the cell surface. Without wishing to be bound by theory, this upregulation is due to the normal compensatory anti-inflammatory immune response. This compensatory response acts as a balance and is necessary to prevent an unchecked immune response, which can damage healthy tissue leading to complications and poor health outcomes. (Panel A) Individuals who have chronic inflammation have elevated circulating levels of anti-inflammatory immune cells. This chronic compensatory anti-inflammatory response leads to immune dysregulation and can predispose individuals to certain diseases. Without wishing to be bound by theory, GPR84 expression naturally dampens the immune response, preventing over activation and collateral damage to healthy tissue. This occurs through interaction with MCFAs, GPR84's natural ligand. MCFA-GPR84 interaction places immunes cells into a less-glycolytic state and consequently, into an anti-inflammatory phenotype. While the mechanism evidence remains to be elucidated, this pairs with the existing understanding that anti-inflammatory immune cells rely more on fatty acid oxidation. (Panel B)

FIG. 2 is a schematic showing (Panel A) the use of a GPR84 agonist to promote and anti-inflammatory state. As described in FIG. 2 Panel B, many fats are already associated with immunosuppression. These immunosuppressive phenotypes can come from the effects of MCFAs in the diet and body. Without wishing to be bound by theory, GPR84 agonism will mimic this phenomenon. We will see an increase in the activity of immunosuppressive immune cells as well as decreases in activity from inflammatory immune cells. Administration of an agonist can be useful in the treatments of inflammatory diseases. Panel B shows that, without wishing to be bound by theory, use of GPR84 antagonists promotes the inflammatory state. Our findings described herein are the reverse as stated in Panel A. Here we describe how blocking GPR84 will prevent the MCFA signaling pathway. This will place cells into a more active and pro-inflammatory state and can be useful in the treatment of cancers and other diseases which require activated immune responses.

FIG. 3 shows a schematic depicting GPR84 FFAR is increased in mouse and human myeloid-derived suppressor cells. Panel A demonstrates that bone marrow derived mouse myeloid-derived suppressor cells (BM-MDSC) has an increased expression of GPR84 FFAR after 4 days in culture with GM-CSF and IL-6. Panel B indicates that human MDSC express GPR84. In this case Granulocytic-MDSC from patients with severe COVID-19 have an increased expression of RNA encoding for GPR84. This means that as disease worsens in severity, expression of GPR84 on associated MDSCs increases. Without wishing to be bound by theory, this is due to the natural attempt to try and dampen the severe immune response being mounted against virus.

FIG. 4 shows a Western blot and graphs of the effects of agonists and antagonists of GPR84 on myeloid cells. Effects of antagonist and agonist on protein expression from bone marrow differentiated MDSCs. (Panel A) Bone marrow derived murine MDSC show expression of arginase 1 (Lane 1 and 2) and a small but detectable expression of iNOS. MDSC were also treated with descending concentrations the GPR84 antagonist (GLPG1205) or agonists (DL175). The results indicate that higher concentrations of the antagonist GLPG1205 decreases the expression of Arginase 1 protein, while the opposite is true with the agonist DL175. Increasing concentrations of the agonist DL175 increases the expression of Arginase-1 and iNOS. These same results are graphed in Panel B and Panel C.

FIG. 5 shows a Western blot and graphs of antagonist and agonist on the expression of proteins associated with the killer function of CD3 T cells. T cells were activated using an antibody cocktail consisting of anti-CD3 and anti-CD28. T cells were also treated with descending concentrations of both GPR84 antagonist (GLPG1205) or agonists (DL175). Relative protein abundance of the two main proteins responsible for the killer function of T cells, namely Granzyme B and Perforin were measured by western blot. Results indicate that at the high doses, use of the antagonist dramatically increases the expression of Granzyme B and perforin (Panel A). Panel B is the relative abundance of Granzyme B, and Panel C is the abundance of Perform.

FIG. 6 shows graphs of functional effects of GPR84 agonist and antagonist on MDSCs and T cells. Panel A shows that adding the GPR84 antagonist GLPG1205 into a mixed culture of myeloid derived suppressor cells (MDSC) and T cells, allows T cells to proliferate (multiply) better (last two bars on Panel A). This happens because the antagonist reduces Arginase 1 (as shown in Panel A) which in turn reduces the suppressive capacity of MDSCs. Panel B shows that the use of an agonist did not have a statistical effect on the MDSC suppressive capacity, neither enhancing nor reducing T cell proliferation. Panel C shows that when CD8 cytotoxic T cells are treated with the GPR84 antagonists they are better killers (higher red lines) as compared to the untreated cells (black lines). This can be explained by the increased expression of Granzyme B and perforin. The reverse was found when using the GPR84 agonist, with CD8 T cells having a reduced cytotoxic capacity (lower blue line) and weakened ability to kill antigen specific tumor cells.

FIG. 7 shows graphs of the effect of GPR84 agonist and antagonists on T cell proliferation (cell multiplication). The rate of proliferation of the two T cells subtypes, the cytotoxic CD8 T cell or the helper CD4 T cell were examined. There was a significant increase in proliferation in CD8 T cells treated with the antagonist, and a significant decrease in proliferation in T cells treated with the agonist. (Panel A) In CD4 T cells, there was no effect from the antagonist, but there was a significant reduction in proliferation in T cells treated with the agonist. (Panel B) Statistically significance is identified using the star symbol. P-value was input in conditions that did not provide a significant difference.

FIG. 8 shows graphs of the effects of GPR84 agonist and antagonists on T cell cytokine production. Cytokine production was measured using a 32-cytokine multiplex. Results indicate an overall increase in cytokine secretion in CD3 T cells treated with the GPR84 antagonist, and an overall reduction in T cells treated with the GPR84 agonist. Statistically significance is identified using the star symbol. P-value was input in conditions that did not provide a significant difference. More importantly, as shown in FIG. 9, the production of Interferon gamma (IFNγ) was highly increased by antagonists and significantly decreased by agonists of GPR84.

FIG. 9 shows graphs of effects of GPR84 agonist and antagonists on T cell maturation. Maturation of cytotoxic CD8 T cells were examined by flow cytometry. CD8 T cells treated with the GPR84 antagonists had increases in cell markers of maturation (LAG-3, CTLA4, PD-1), activation (CD69, CD25), and cytotoxic function (Granzyme B and Perform). In turn, use of the GPR84 agonist had opposite effect, with CD8 T cells having a larger anti-inflammatory phenotype (Panel A). These findings were also found when looking at the total abundance (MFI or Mean fluorescence intensity) of these markers in CD8 T cells. (Panel B). Statistically significance is identified using the star symbol. P-value was input in conditions that did not provide a significant difference.

FIG. 10 shows effect of GPR84 agonist and antagonists on T cell maturation. Maturation of helper CD4 T cells were examined by flow cytometry. CD4 T cells treated with the GPR84 antagonists had increases in maturation, activation, and cytotoxic expression. In turn, use of the GPR84 agonist had opposite effect, with CD4 T cells having a reduced inflammatory phenotype (Panel A). These findings were also found when looking at the total abundance of these markers in CD4 T cells. (Panel B). Statistically significance is identified using the star symbol. P-value was input in conditions that did not provide a significant difference.

FIG. 11 shows graphs of effects of GPR84 agonist and antagonists on T cell maturation and long-lived memory induction. Memory T cell induction and naïve T cell presence in both CD8 and CD4 T cells were determined by flow cytometry. Panel A demonstrates that the use of the antagonist induced more effector memory T cells in both CD8 and CD4 T cells, which indicates long-lived immunity. When examining the maturation of these T cells, use of the antagonist reduced the percentage of naïve CD8 T cells, indicating a maturation of this T cell subset. (Panel B) Statistically significance is identified using the star symbol. P-value was input in conditions that did not provide a significant difference.

FIG. 12 shows graphs of effects of GPR84 agonist and antagonists on T cell metabolism. Glycolytic metabolism was measured using a seahorse assay and determining the extracellular acidification rate (ECAR) as well as the proton efflux rate (PER). CD3 T cells treated with the GPR84 antagonists had elevated ECAR (Panel A) and PER (Panel B) rates signifying greater use of the glycolytic pathway. The opposite was found when using the high dose of the GPR84 agonist. The increased use of glucose by T cells stimulated with the antagonist is a partial explanation of why they have an increased killer and cytokine production functions.

FIG. 13 shows graphs of effects of GPR84 agonist and antagonists on T cell glucose uptake. The importance of the increased use of glucose by T cells is shown by the fact that when one stimulates T cells with the antagonist, the increase the uptake of Fluorescent glucose (2-NBDG) This is shown for CD8 (Panel A) and CD4 (Panel B) T cells were found to increase their glucose consumption after treatment with GPR84 antagonists. This was measured both as the total number of 2-NBDG positive T cells, as well as the relative abundance of glucose taken up by T cells (MFI). In GPR84 agonist treated T cells, we found a reduction in glucose uptake. Statistically significance is identified using the star symbol.

FIG. 14 shows graphs of gene expression of each enzyme used in the metabolism of glucose after 48-hour treatment with GPR84 agonist or antagonist. This is further corroborated by the fact that the use of a GPR84 antagonist increased the gene expression of multiple enzymes used in glucose metabolism and an opposite effect was found when using the GPR84 agonist. Statistically significance is identified using the star symbol. P-value was input in conditions that did not provide a significant difference.

FIG. 15 shows representative data that indicate GPR84 receptor modulation alters proliferation and cytotoxic activity of CD8 T cells. (Panel A) Representative blot probed with anti-GPR84 and anti-β-actin. Samples consist of naïve CD3 T cells and activated CD3 T cells treated for 72 hours. (Panel B) Representative histogram and average proliferation from three independent biological replicates. Naïve CD8 T cells were labeled with CFSE, activated and treated for 72 hours. CD8 T cell proliferation was determined by CFSE dilution. Averages are displayed as the average Log₂ fold change compared DMSO control. (Panel C) Representative blot probed for anti-granzyme B, anti-perforin and anti-β-actin. Samples consist of naïve murine CD3 T cells activated and treated for 72 hours. Granzyme B (Panel D) and perforin (Panel E) protein quantification normalized to the housekeeping control β-actin. Quantification is displayed as the average Log₂ fold change compared DMSO control from three independent biological replicates. (Panel F) Blot probed for anti-granzyme B, anti-perforin and anti-β-actin. Sample consists of naïve human CD3 T cells activated and treated for 72 hours. (Panel G) Representative histogram and average cytotoxicity from three independent biological replicates. Naïve CD8 T cells were activated and treated for 72 hours. Treated CD8 were added to a 1:1 ratio of CFSE labeled non-target (EL4) and target (E.G7-OVA) cells at an effector to target (E:T) ratio of 3:1, 1:1, or 0.3:1. Analysis is displayed as the average percent specific lysis of the target cell. For experiments displayed, naïve T cells were activated with anti-CD3/anti-CD28 and were either left untreated (NT), or treated with DMSO, 3 μM or 10 μM GPR84 antagonist GLPG1205, or 3 μM or 10 μM GPR84 agonist DL175. Results are displayed as the mean with standard deviation. Statistical significance was performed by unpaired two-tailed t-test comparing the treatment group to DMSO control. Significance was defined by: *p≤0.05, **p≤0.01, ***p≤0.001, ****p≤0.0001.

FIG. 16 shows graphs of representative data that indicate GPR84 receptor modulation reduces apoptosis in CD8 T cells. Percent apoptotic cells were examined by flow cytometry from activated CD8 (Panel A) and CD4 (Panel B) T cells treated for 72 hours. Results are displayed as the percent apoptotic cells from DMSO, 3 μM or 10 μM GPR84 antagonist GLPG1205, or 3 μM or 10 μM GPR84 agonist DL175 treated samples from five independent biological replicates. Results are displayed as the mean with standard deviation. Statistical significance was performed by unpaired two-tailed t-test comparing the treatment group to DMSO control. Significance was defined by: *p≤0.05, **p≤0.01.

FIG. 17 shows graphs and blots of representative data that indicate GPR84 receptor modulation alters the suppressive capacity of BM-MDSCs. (Panel A) Representative blot probed with anti-GPR84 and anti-β-actin. Samples consist of naïve bone marrow and differentiated BM-MDSCs treated for 72 hours. (Panel B) Representative blot probed for anti-arginase-I, anti-iNOS and anti-β-actin. Samples consist of differentiated BM-MDSCs activated and treated for 72 hours. Arginase-I (Panel C) and iNOS (Panel D) quantification normalized to the housekeeping control β-actin. Quantification is displayed as the average Log₂ fold change compared DMSO control of four independent biological replicates. (Panel E) Suppression assay measuring the percent proliferation of CD3 T cells co-cultured with treated BM-MDSCs for 72 hours. For experiments displayed, bone marrow cells were differentiated with 40 ng/ml GM-CSF, G-CSF, and IL-6 for 96 hours. BM-MDCSs were treated for 72 hours and began 24 hours after the start of differentiation. Cells were either left untreated (NT), or treated with DMSO, 3 μM or 10 μM GPR84 antagonist GLPG1205, or 3 μM or 10 μM GPR84 agonist DL175. Results are displayed as the mean with standard deviation of four independent biological replicates. Suppression assay biological replicates were conducted each with 3-6 technical replicates. Statistical significance was performed by unpaired two-tailed t-test comparing the treatment group to DMSO control. Significance was defined by: *p≤0.05, **p≤0.01, ***p≤0.001.

FIG. 18 shows flow cytometry data that indicates GPR84 receptor modulation regulates CD8 T cell activation. Activation markers CD69 (Panel A), CD25 (Panel B), CTLA-4 (Panel C), LAG-3 (Panel D), PD-1 (Panel E), and IFN-γ (Panel F) were examined by flow cytometry from activated CD8 T cells treated for 72 hours. Representative pseudocolor plots of DMSO, 10 μM GLPG1205, and 10 μM DL175 treated samples. Results are displayed as the average Log₂ fold change compared DMSO control from five independent biological replicates. For experiments displayed, naïve T cells were activated with anti-CD3/anti-CD28 and were treated with DMSO, 3 μM or 10 μM GPR84 antagonist GLPG1205, or 3 μM or 10 μM GPR84 agonist DL175. Results are displayed as the mean with standard deviation. Statistical significance was performed by unpaired two-tailed t-test comparing the treatment group to DMSO control. Significance was defined by: *p≤0.05, **p≤0.01, ***p≤0.001, ****p≤0.0001.

FIG. 19 shows flow cytometry data that indicates GPR84 receptor modulation regulates CD4 T cell activation. Activation markers CD25 (Panel A) and CTLA-4 (Panel B) were examined by flow cytometry from activated CD4 T cells treated for 72 hours. Representative pseudocolor plots of DMSO, 10 μM GLPG1205, and 10 μM DL175 treated samples. Results are displayed as the average Log₂ fold change compared DMSO control from five independent biological replicates. For experiments displayed, naïve T cells were activated with anti-CD3/anti-CD28 and were treated with DMSO, 3 μM or 10 μM GPR84 antagonist GLPG1205, or 3 μM or 10 μM GPR84 agonist DL175. Results are displayed as the mean with standard deviation. Statistical significance was performed by unpaired two-tailed t-test comparing the treatment group to DMSO control. Significance was defined by: *p≤0.05, **p≤0.01.

FIG. 20 shows representative data that indicates GPR84 receptor modulation regulates T cell cytokine secretion. Inflammatory, mitogenic, and T cell differentiating cytokines and chemokines IL-2 (Panel A), IL-15 (Panel B), IL-9 (Panel C), IL-12(p40) (Panel D), TNFα (Panel E), G-CSF (Panel F), GM-CSF (Panel G), IL-1P (Panel H), IP-10 (Panel I), IL-6 (Panel J), Eotaxin (Panel K), LIF (Panel L), LIX (Panel M), IL-10 (Panel N), IL-4 (Panel O), IL-3 (Panel P), MCP-1 (Panel Q), VEGF (Panel R), KC (Panel S), RANTES (Panel T) were measured from the supernatant of activated CD3 T cells treated for 72 hours. Results are displayed as the average Log₂ fold change compared DMSO control from five independent biological replicates. For experiments displayed, naïve T cells were activated with anti-CD3/anti-CD28 and were treated with DMSO, 3 μM or 10 μM GPR84 antagonist GLPG1205, or 3 μM or 10 μM GPR84 agonist DL175. Results are displayed as the mean with standard deviation. Statistical significance was performed by unpaired two-tailed t-test comparing the treatment group to DMSO control. Significance was defined by: *p≤0.05, **p≤0.01, ***p≤0.001, ****p≤0.0001.

FIG. 21 shows representative data that indicates GPR84 receptor modulation regulates CD8 T cell differentiation. (Panel A) Representative pseudocolor plots of DMSO, 10 μM GLPG1205, and 10 μM DL175 treated samples. (Panel B) Naïve CD8 T cells defined by the cell surface markers CD62L+ CD44− and (Panel C) effector/effector memory by CD62L− CD44+. Differentiated CD8 T cells were examined by flow cytometry after treatment for 72 hours. Results are displayed as the average Log₂ fold change compared DMSO control from five independent biological replicates. For experiments displayed, naïve T cells were activated with anti-CD3/anti-CD28 and were treated with DMSO, 3 μM or 10 μM GPR84 antagonist GLPG1205, or 3 μM or 10 μM GPR84 agonist DL175. Results are displayed as the mean with standard deviation. Statistical significance was performed by unpaired two-tailed t-test comparing the treatment group to DMSO control. Significance was defined by: *p≤0.05, **p≤0.01, ***p≤0.001.

FIG. 22 shows representative data that indicates GPR84 receptor modulation regulates CD4 T cell differentiation. (Panel A) Representative pseudocolor plots of DMSO, 10 μM GLPG1205, and 10 μM DL175 treated samples. (Panel B) Naïve CD4 T cells defined by the cell surface markers CD62L+ CD44− and (Panel C) effector/effector memory by CD62L-CD44+. Differentiated CD4 T cells were examined by flow cytometry after treatment for 72 hours. Results are displayed as the average Log₂ fold change compared DMSO control from five independent biological replicates. For experiments displayed, naïve T cells were activated with anti-CD3/anti-CD28 and were treated with DMSO, 3 μM or 10 μM GPR84 antagonist GLPG1205, or 3 μM or 10 μM GPR84 agonist DL175. Results are displayed as the mean with standard deviation. Statistical significance was performed by unpaired two-tailed t-test comparing the treatment group to DMSO control. Significance was defined by: *p≤0.05, **p≤0.01.

FIG. 23 shows representative data that indicates GPR84 receptor knockout by CRISPR-Cas9 enhances CD8 T cell cytotoxic capacity. GPR84 receptor was removed in CD8 pmel-I T cells using CRISPR-Cas9. Displayed histogram and cytotoxicity are representative from two independent biological replicates. Activated CD8 were added to a 1:1 ratio of CFSE labeled non-target (EL4_{control}) and target (EL4_{gp100}) cells at an effector to target (E:T) ratio of 3:1, 1:1, or 0.3:1. Analysis is displayed as the average percent specific lysis of the target cell. Statistical significance was performed by two-way ANOVA comparing GPR84 knockout (crGPR84) and target control (crNTC). Results are displayed as the mean with standard deviation. Significance was defined by: **p≤0.01, ***p≤0.001.

FIG. 24 shows representative data that indicates GPR84 receptor modulation metabolically reprograms CD8 T cells. (Panel A) OCR, (Panel B) ECAR and (Panel C) PER rates during an XFe ATP rate assay. Representative plots from are from naïve CD8 T cells activated with anti-CD3/anti-CD28 and treated with either DMSO, 10 μM GLPG1205, or 10 μM DL175 for 72 hours. Each condition was analyzed using 6-8 technical replicates. (Panel D) Average total ATP production rate by each treatment condition. (Panel E) Stratification of the average glycolysis ATP production rate (glycoATP) and average mitochondrial ATP production rate (mitoATP). (Panel F) Percent of ATP generated by glycolysis versus mitochondrial OXPHOS. (Panel G) Energetic map representing the associated energetic phenotype by plotting the mitoATP production rate versus glycoATP production rate. Displayed results are representative from two independent biological replicates. Results are displayed as the mean with standard deviation. Statistical significance was performed by unpaired two-tailed t-test comparing all treatment combinations. (Panel H) Mean fluorescent intensity of glucose analog 2-NBDG uptake in activated CD8 T cells treated for 72 hours. Results are displayed as the average Log₂ fold change with standard deviation compared DMSO control from four independent biological replicates. (Panels I-L) Quantitative PCR of naïve CD8 T cells activated with anti-CD3/anti-CD28 and treated with either DMSO, 3 μM or 10 μM GPR84 antagonist GLPG1205, or 3 μM or 10 μM GPR84 agonist DL175 for 48 hours. Results are displayed as the average Log₂ fold change with standard deviation compared to DMSO control from three independent biological replicates. Significance was defined by: *p≤0.05, **p≤0.01, ***p≤0.001, ****p≤0.0001.

FIG. 25 shows representative data that indicates GPR84 receptor modulation metabolically reprograms BM-MDSCs. (Panel A) OCR, (Panel B) ECAR and (Panel C) PER rates during an XFe ATP rate assay. Representative plots from are from BM-MDSCs differentiated with 40 ng/ml GM-CSF, G-CSF, and IL-6 and treated with either DMSO, 10 μM GLPG1205, or 10 μM DL175 for 72 hours. Each condition was analyzed using 6-8 technical replicates. (Panel D) Average total ATP production rate by each treatment condition. (Panel E) Stratification of the average glycolysis ATP production rate (glycoATP) and average mitochondrial ATP production rate (mitoATP). (Panel F) Percent of ATP generated by glycolysis versus mitochondrial OXPHOS. (Panel G) Energetic map representing the associated energetic phenotype by plotting the mitoATP production rate versus glycoATP production rate. Displayed results are representative from one independent biological replicate. Results are displayed as the mean with standard deviation. Statistical significance was performed by unpaired two-tailed t-test comparing all treatment combinations. Significance was defined by: *p≤0.05, **p≤0.01, ***p≤0.001, ****p≤0.0001.

FIG. 26 shows representative data that indicates GPR84 receptor modulation of CD8 T cells reveals differentiation, metabolic and effector genomic changes by single-cell RNA sequencing. (Panel A) UMAP plots of an unsupervised clustering consisting of a mixture of naïve CD3 T cells activated with anti-CD3/anti-CD28 and differentiated BM-MDSCs treated with either DMSO, 10 μM GLPG1205, or 10 μM DL175 for 16 hours. Isolation of T lymphocyte compartment by CD3 UMAP refinement and reclustering. (Panel B) Isolation of CD8 T cell compartment by CD8 UMAP refinement and reclustering. (Panel C) Visualized gene expression heatmap of a list of genes used to characterize the effector, early-effector, and naïve CD8 T cell subsets. (Panel D) UMAP plots of genes associated with characterized CD8 T cell subsets. (Panel E) Bar chart representing the total number of cells quantified in each treatment group. (Panel F) Pie-chart representing the percentage of effector, early-effector, and naïve CD8 T cell subsets in each treatment group. Gene expression heatmap of a curated list of genes associated with glucose metabolism (Panel G), fatty acid metabolism, lipid metabolism (Panel H), and cytotoxicity (Panel I). Heat map visualizations represents the average gene expression changes between treatment groups.

FIG. 27 shows representative data that indicates pre-treatment of adoptively transferred CD8 T cells with GPR84 antagonist GLPG1205 confers an enhanced in vivo anti-tumor response. (Panel A) Timeline of adoptive transfer experiments. (Panel B) Average tumor volumes and associated spider plots of B16-OVA tumor bearing mice treated with sham, DMSO pre-treated, or 10 μM GLPG1205 pre-treated CD8 OT-I adoptive transfer. Displayed results are pooled from two independently significant experiments. Total pooled mice per group: sham (n=15), DMSO (n=19), 10 μM GLPG1205 (n=18). (Panel C) Representative pseudocolor plots of intratumoral SIINFEKL tetramer (SEQ ID NO: 1) positive CD8 T cells from sham, DMSO, and 10 μM GLPG1205 groups. Results are displayed as the average SIINFEKL tetramer (SEQ ID NO: 1) positive CD8 T cells from sham (n=4), DMSO pre-treated (n=7), or 10 μM GLPG1205 pre-treated (n=6) adoptive transfer. (Panel D) Average tumor volumes and associated spider plots of B16 tumor bearing mice treated with sham, DMSO pre-treated, or 10 μM GLPG1205 pre-treated CD8 pmel-I adoptive transfer. Displayed results are of one significant experiment. Total mice per group: sham (n=5), DMSO (n=10), 10 μM GLPG1205 (n=10). Statistical significance was performed by unpaired two-tailed t-test comparing all treatment combinations. SIINFEKL tetramer (SEQ ID NO: 1) positive results are displayed as the mean with standard deviation. Tumor volume growth rates are displayed as the mean with standard error. Significance was defined by: *p≤0.05, **p≤0.01, ***p≤0.001.

FIG. 28 shows representative data that indicates pre-treatment of adoptively transferred CD8 T cells with GPR84 agonist DL175 confers an enhanced in vivo pro-tumor response. (Panel A) Timeline of adoptive transfer experiments. (Panel B) Average tumor volumes and associated spider plots of B16-OVA tumor bearing mice treated with sham, DMSO pre-treated, or 10 μM DL175 pre-treated CD8 OT-I adoptive transfer. Displayed results are pooled from one significant experiment. Total pooled mice per group: sham (n=4), DMSO (n=10), 10 μM DL175 (n=9). Statistical significance was performed by two-way ANOVA with Dunnett's post-hoc test. Tumor volume growth rates are displayed as the mean with standard error. Significance was defined by: *p≤0.05, **p≤0.01

FIG. 29 shows a dosing timeline and data that indicates in vivo titration of SIINFEKL (SEQ ID NO: 1) to achieve a suboptimal vaccination response. (Panel A) Timeline of adoptive transfer experiments. (Panel B) Average percent proliferation of adoptively transferred naïve CFSE labeled CD8 OT-I T cells vaccinated with titrated doses of SIINFEKL (SEQ ID NO: 1) in an IFA emulsion. Total mice per group: no vaccination (n=4) vaccinated (n=3). Sub-optimal dose was identified as the amount of antigen which elicited a 50% proliferation response rate.

FIG. 30 shows a dosing timeline and data that indicates GPR84 antagonist treatment improves in vivo vaccination response. (Panel A) Timeline of adoptive transfer experiments. Analysis is from the lymph node or spleen from mice adoptively transferred with naïve CFSE labeled CD8 OT-I T cells, vaccinated with 0.07 g SIINFEKL (SEQ ID NO: 1) in an IFA emulsion, and orally treated with either 30 mg/kg or 90 mg/kg GLPG1205 once daily for three days. Displayed results are pooled from two independent experiments. Total pooled mice per group: No treatment (n=2), Vehicle (n=10), 30 mg/kg GLPG1205 (n=10), 90 mg/kg GLPG1205 (n=10). Average percent proliferation (Panel B) and PD-1 expression (Panel C) of adoptively transferred CD8 OT-I T cells in lymph node compartment. Average percent proliferation (Panel D) and PD-1 expression (Panel E) of adoptively transferred CD8 OT-I T cells in splenic compartment. Results are displayed as the mean with standard deviation. Statistical significance was performed by unpaired two-tailed t-test comparing the treatment groups to vehicle control. Significance was defined by: *p≤0.05, **p≤0.01, ***p≤0.001, ****p≤0.0001.

FIG. 31 shows representative data that indicates GPR84 antagonist treatment enhances T cell differentiation after in vivo vaccination. Analysis from the lymph node or spleen from mice adoptively transferred with naïve CFSE labeled CD8 OT-I T cells, vaccinated with 0.07 g SIINFEKL (SEQ ID NO: 1) in an IFA emulsion, and orally treated with either 30 mg/kg or 90 mg/kg GLPG1205 once daily for three days. Naïve OT-I CD8 T cells were defined by the cell surface markers CD62L+ CD44− and effector/memory by CD62L− CD44+. Displayed results are pooled from two independent experiments. Total pooled mice per group: No treatment (n=2), Vehicle (n=10), 30 mg/kg GLPG1205 (n=10), 90 mg/kg GLPG1205 (n=10). Average percent naïve (Panel A) and effector/memory (Panel B) of adoptively transferred CD8 OT-I T cells in lymph node compartment. Average percent naïve (Panel C) and effector/memory (Panel D) of adoptively transferred CD8 OT-I T cells in splenic compartment. Results are displayed as the mean with standard deviation. Statistical significance was performed by unpaired two-tailed t-test comparing the treatment groups to vehicle control. Significance was defined by: *p≤0.05, **p≤0.01, ***p≤0.001.

FIG. 32 shows representative data that indicates GPR84 antagonist treatment enhances in vivo anti-tumor therapeutic vaccine efficacy. (Panel A) Timeline of adoptive transfer experiments. (Panel B) Average tumor volumes and associated spider plots of B16-OVA. Displayed results are from one significant independent experiment. Total mice per group: Vehicle (n=6), 30 mg/kg GLPG1205 (n=7). Statistical significance was performed by unpaired two-tailed t-test comparing the treatment group to vehicle control. Significance was defined by: *p≤0.05. Figure discloses “SIINFEKL” as SEQ ID NO: 1.

FIG. 33 shows a graph of GPR84 expression in mouse CD3 cells. Plated cells were activated by plate-bound CD3/28 (1 ug/mL) for 72h prior to lysis and analysis for mRNA expression of GPR84. These data can confirm GPR84 knockout in T cells from GPR84 knockout mice.

FIG. 34 shows blots of markers of T cell activation in GPR84 wild type, heterozygous, and knockout CD3 cells. Plated cells were activated by plate-bound CD3/28 (lug/mL) for 72h prior to lysis and analysis for markers of activation. Cells were treated with indicated. These are two independent experiments showing a similar effect of increased expression of markers of T cell cytotoxicity.

FIG. 35 shows a graph of T cell proliferation. GPR84 wild type, heterozygous, and knockout CD3 T cells were labelled with CFSE and activated with 100 ng/mL CD3/28 for 72h. Analyzed by FACS. This graph indicates that GPR84 knockout enhances T cell proliferation.

FIG. 36 shows a graph of T cell proliferation. Enriched, CFSE-labelled T cells were activated by plate-bound CD3/28 (lug/mL), and treated with indicated concentrations of PBI-4050 for 72h prior to FACS analysis. PBI-4050 (PBI) is a GPR84 antagonist and GPR40 agonist.

FIG. 37 shows a graph of T cell proliferation. Enriched, CFSE-labelled T cells were activated by plate-bound CD3/28 (500 ng/mL) for 72h prior to FACS analysis.

FIG. 38 shows a diagram of free fatty acid receptors. Modified diagram of all free fatty acid receptors, and their associated signaling pathway. GPR84 is a GPCR with a Gai subunit. Signaling inhibits adenylyl cyclase and reduces intracellular cyclic adenosine monophosphate (cAMP). Adapted from Kimura, I., et al., Free Fatty Acid Receptors in Health and Disease. Physiol Rev. 2020.

FIG. 39 shows a modified diagram of the known effects of GPR84 signaling. Observed effects are predominately from the myeloid lineage. GPR84 agonists are reported to enhance a pro-inflammatory response, elevate phagocytosis, and induce organ fibrosis. GPR84 antagonists induce anti-inflammatory effects and reduce organ fibrosis. Adapted from Wojciechowicz, M. L. and A. Ma'ayan, GPR84: an immune response dial? Nat Rev Drug Discov, 2020. 19(6): p. 374 and Chen, L.-H, et al., Modulation of the G-protein coupled receptor 84 (GPR84) by agonist and antagonists. Journal of Medicinal Chemistry.

FIG. 40 shows a modified diagram outlining of the effects (without wishing to be bound by theory) that GPR84 modulation can have on immune cell activity. The two studied immune cells of interest are the T cell and the MDSC. We predict that GPR84 modulation will positively or negatively affect immune cells, which will have implications in the treatment of cancer. Adapted from Wojciechowicz, M. L. and A. Ma'ayan, GPR84: an immune response dial? Nat Rev Drug Discov, 2020. 19(6): p. 374.

FIG. 41 is adapted from Wojciechowicz, Megan L., and Avi Ma'ayan. “GPR84: an immune response dial?” Nature reviews. Drug discovery 6 (2020): 374. This article describes the potential impact that GPR84 can have on modulating the immune system. However, it describes a dial in the opposite direction of our observations. They indicate that using a GPR84 agonist will act as a pro-inflammatory signal and will be useful in the treatment of diseases that require an active immune response, such as cancer. In contrast using an antagonist will lead to an anti-inflammatory response and can be useful in the treatment of certain inflammatory diseases, such as ulcerative colitis, Crohn's disease, and arthritis. This is opposite our observations where using an agonist stimulates anti-inflammatory responses and using and antagonist stimulates an inflammatory protective response.

FIG. 42 shows GPR84 Global KO exhibits a similar effect to GPR84 antagonism through increased cytotoxic protein production in CD8 T Cells. Western blot showing perform and granzyme (left). Perform and granzyme B production in CD8 T Cells.

FIG. 43 shows GPR48 Antagonist and GPR84 Global KO exhibit mechanistic effects through STAT3 in CD8 T Cells. Western blot showing phospho-STAT3 and total STAT3 (left). pSTAT3/tSTAT3 in CD8 T Cells.

FIG. 44 shows GPR84 Global KO exhibits a similar effect to GPR84 antagonism through increased glucose uptake and increased glycolysis pathway gene expression in CD8 T Cells.

FIG. 45 shows GPR84 Global KO exhibits a similar effect to GPR84 antagonism through increased glycolysis, OXPHOS, and ATP Production.

FIG. 46 shows GPR84 Global KO exhibits a similar effect to GPR84 antagonism through increased CD8 T cell proliferation.

FIG. 47 shows GPR84 Global KO exhibits a similar effect to GPR84 antagonism through increased CD8 T cell cytotoxicity in vitro.

FIG. 48 shows GPR84 Global KO exhibits a similar effect to GPR84 antagonism through increased anti-tumor effect in vivo (left) and increased intra-tumoral gp100 tetramer+CD8 T cells (right).

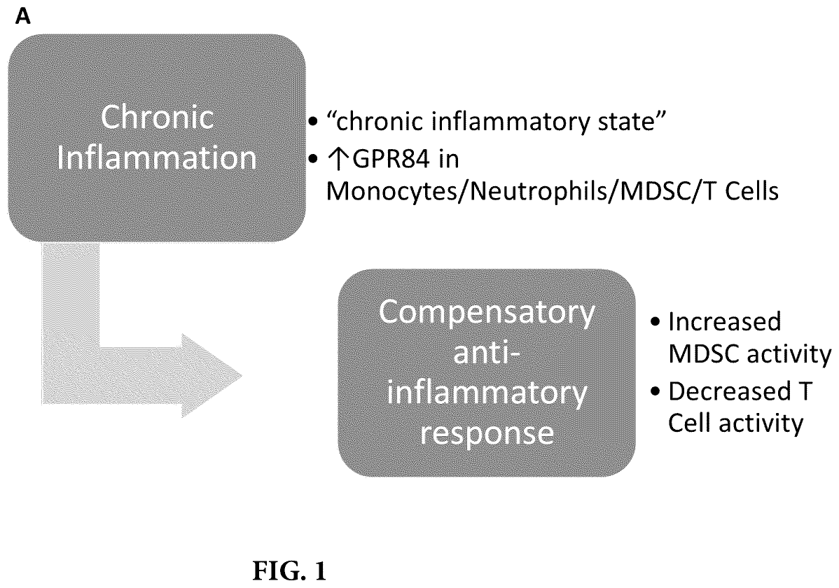

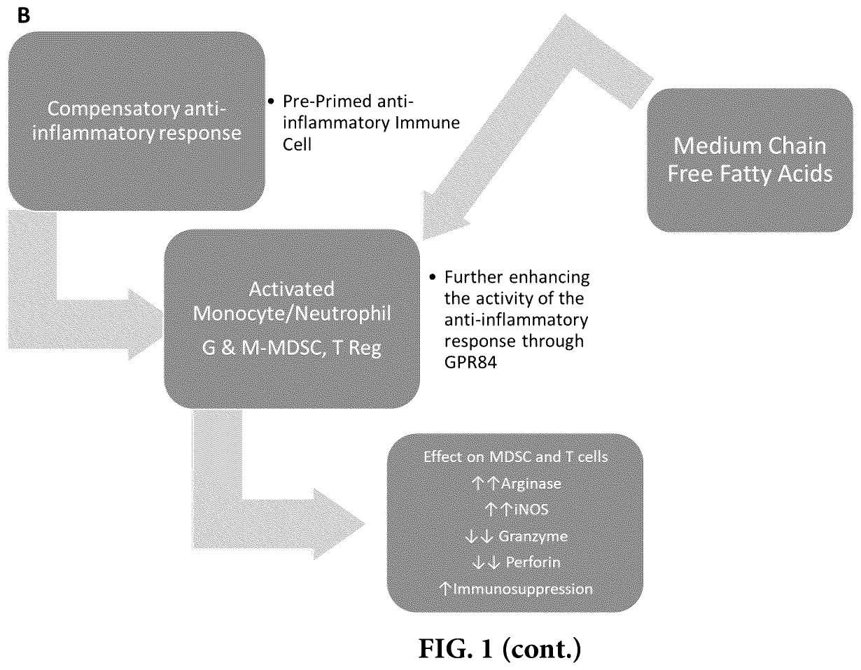

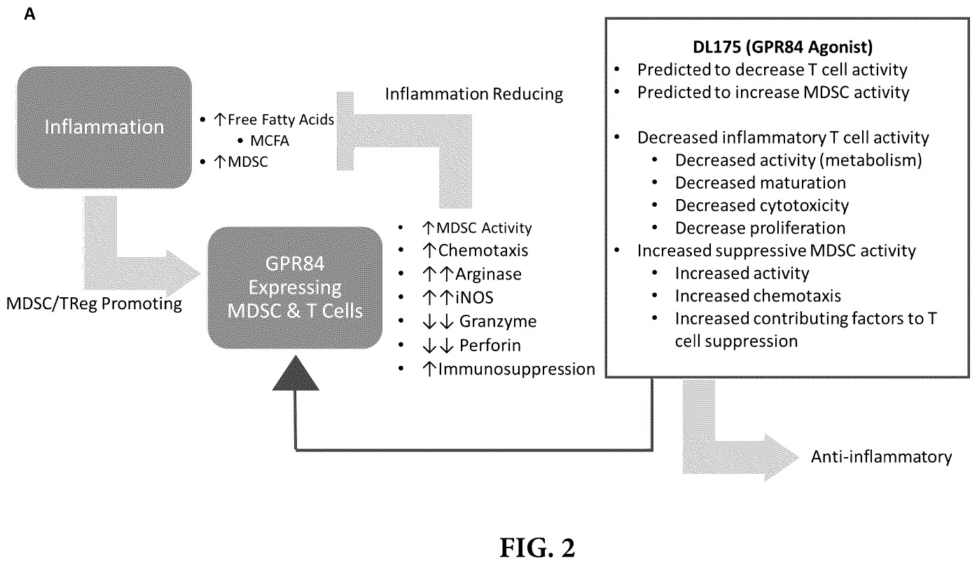

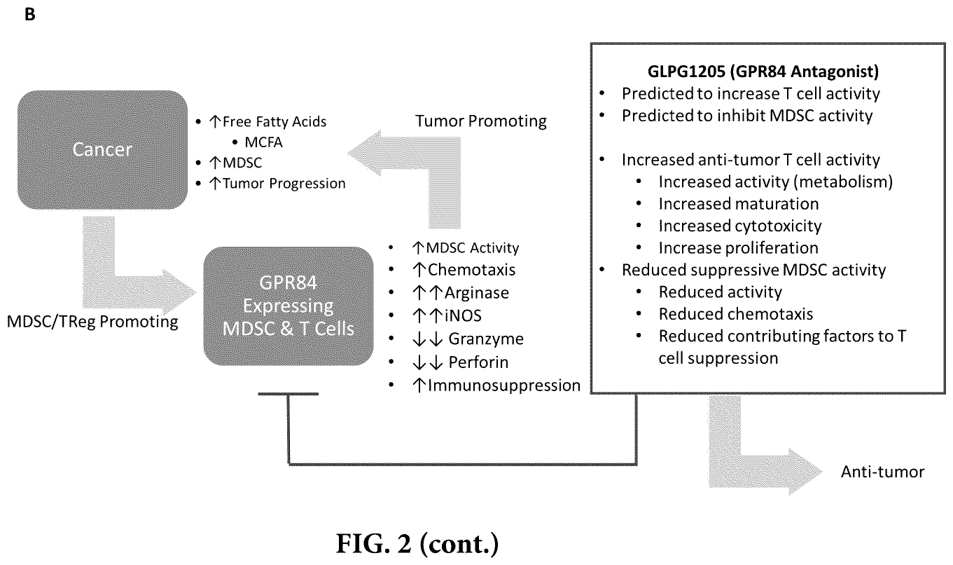

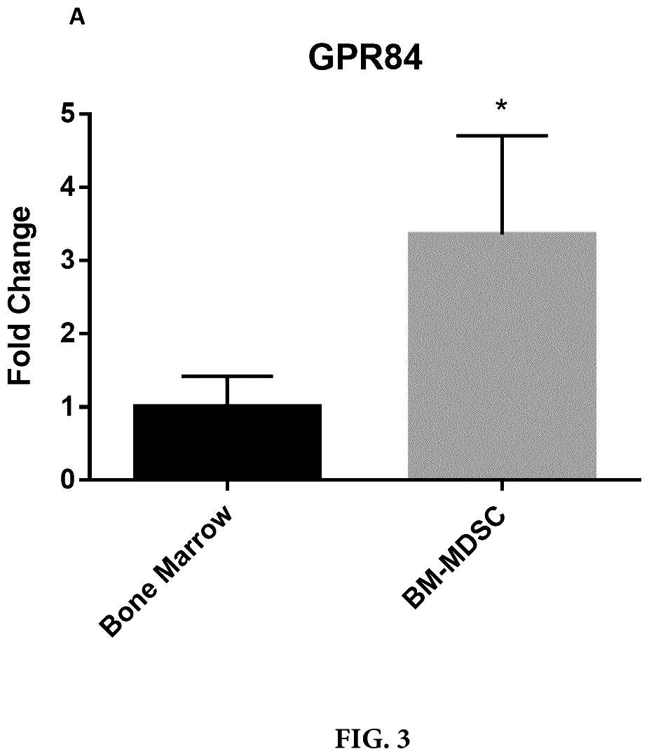

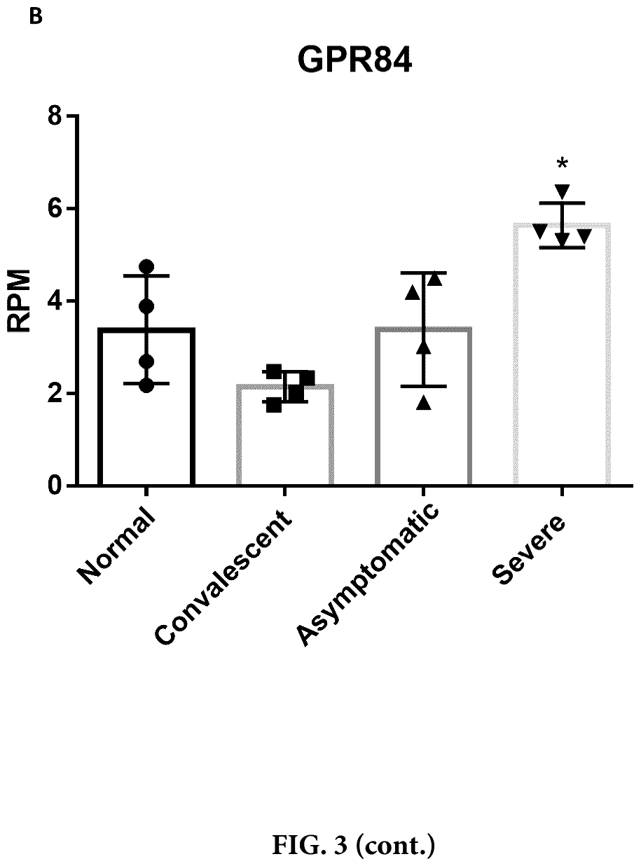

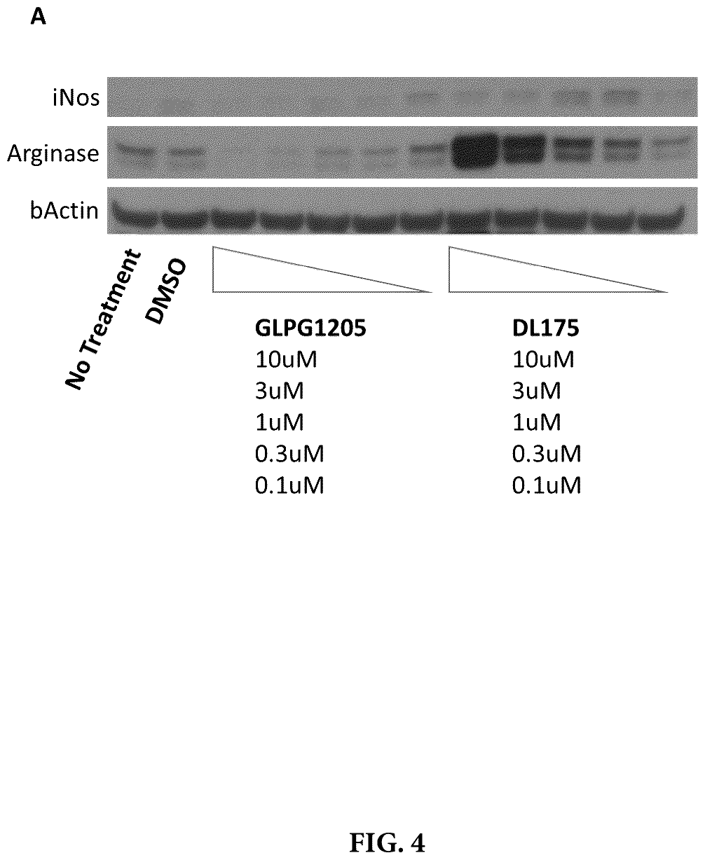

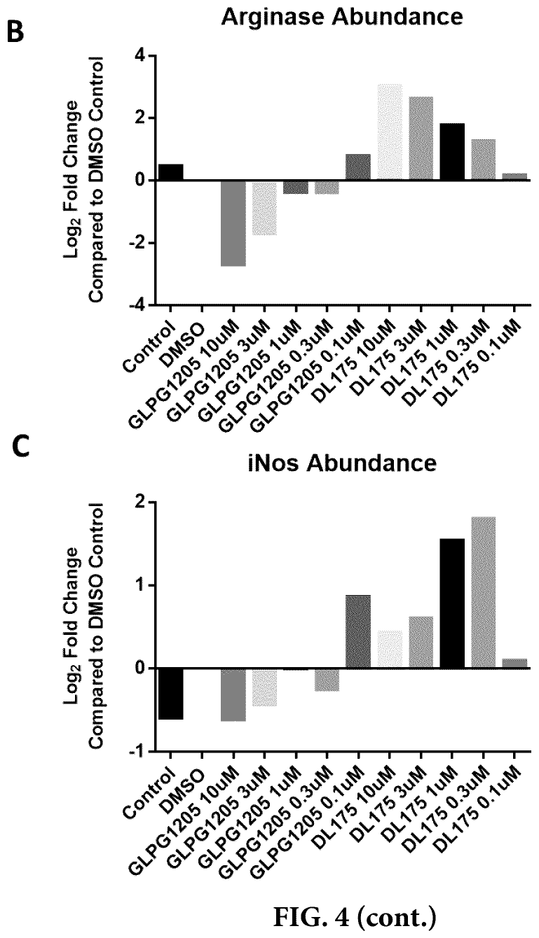

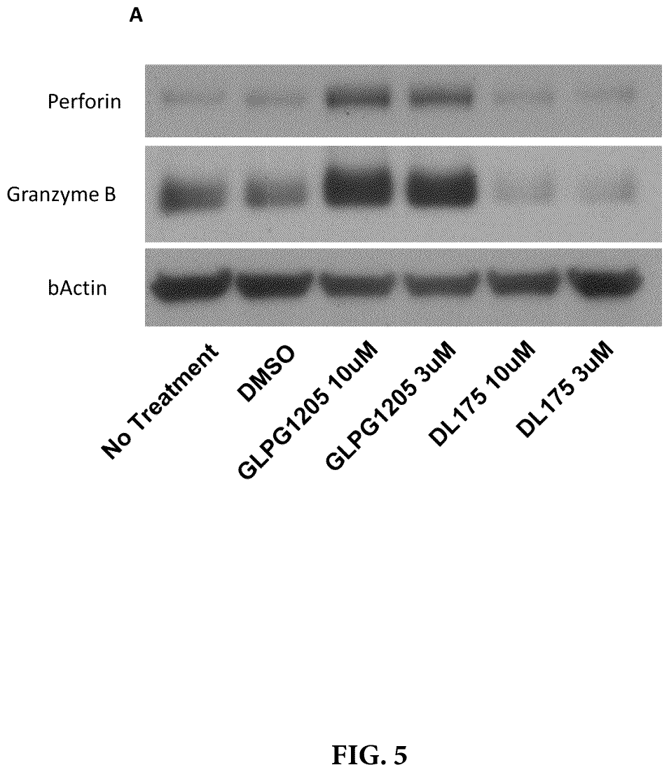

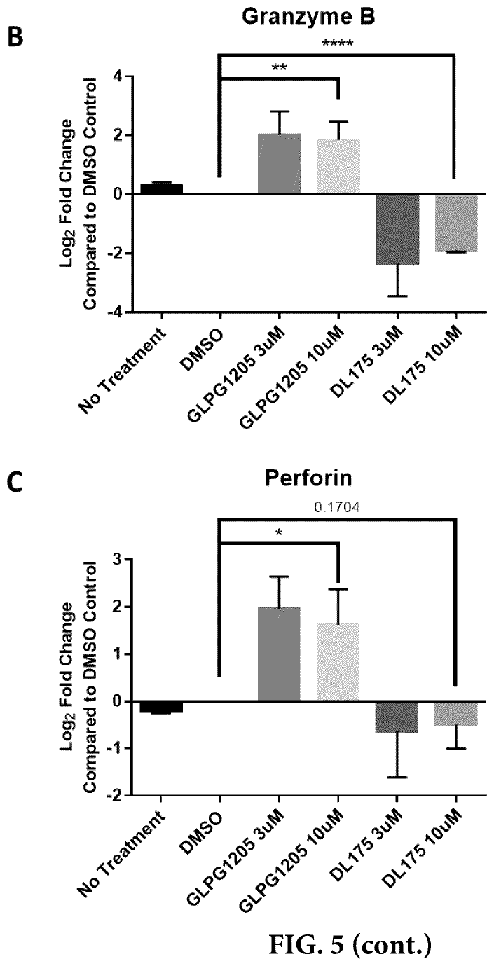

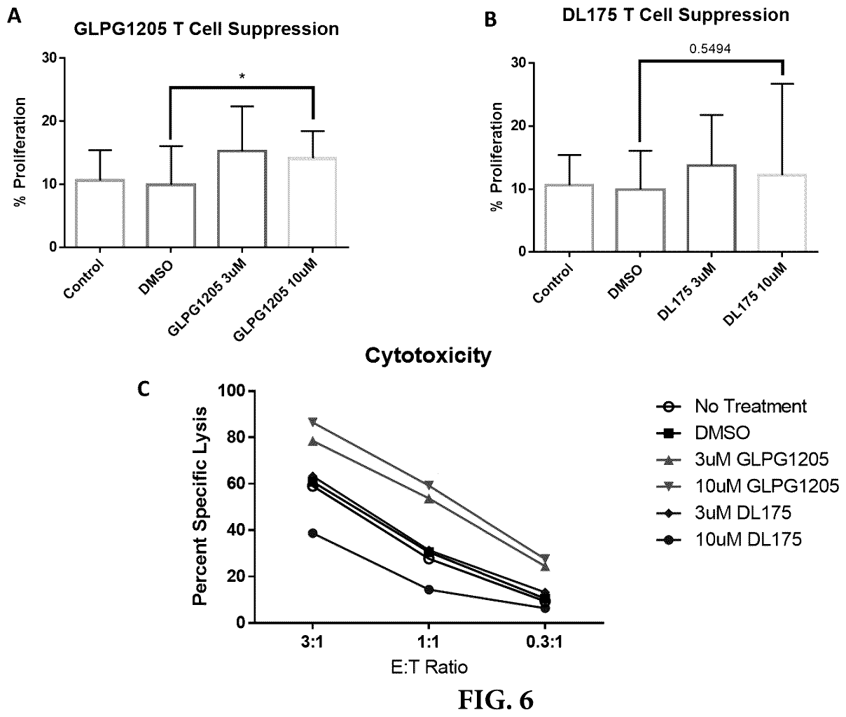

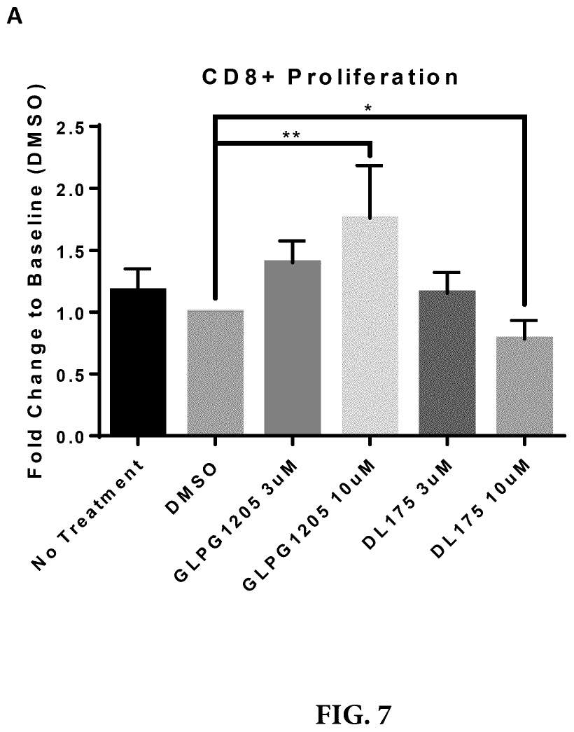

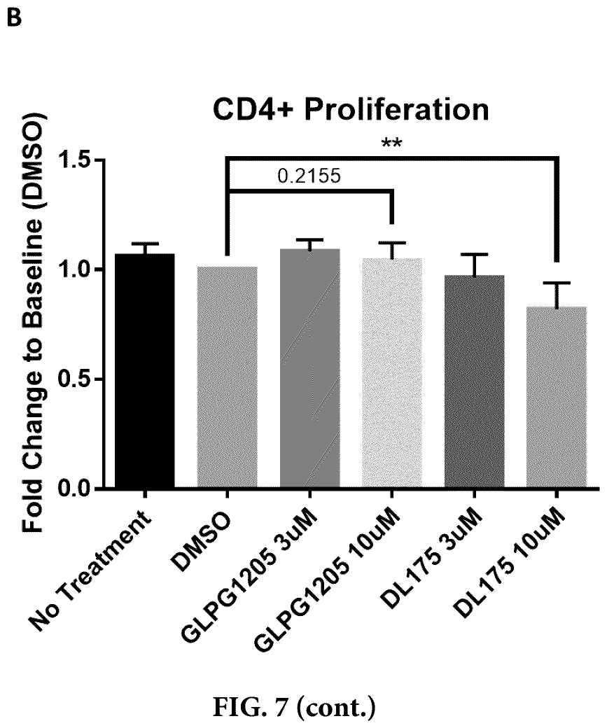

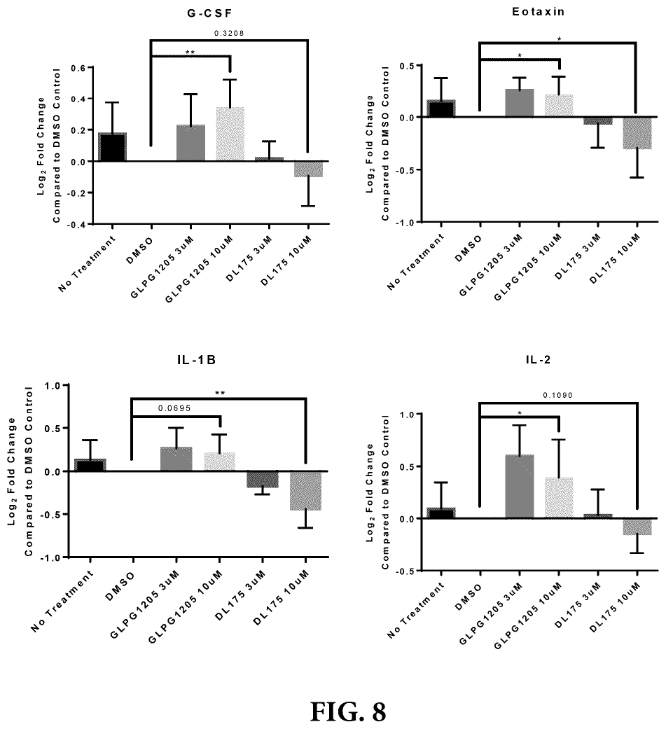

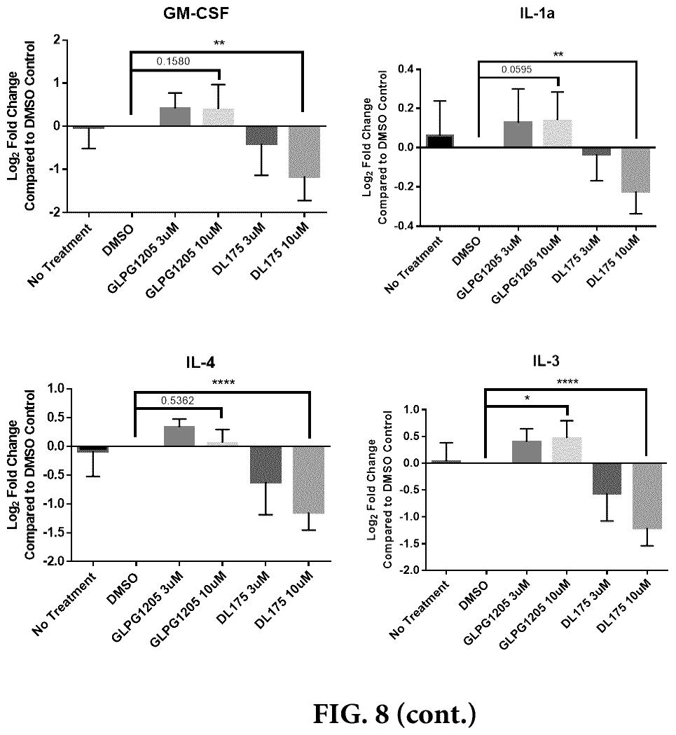

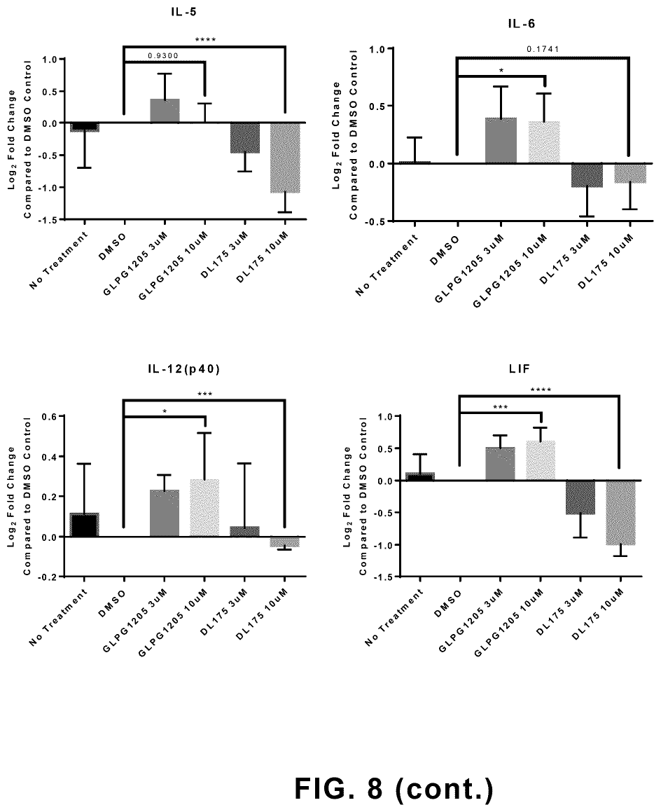

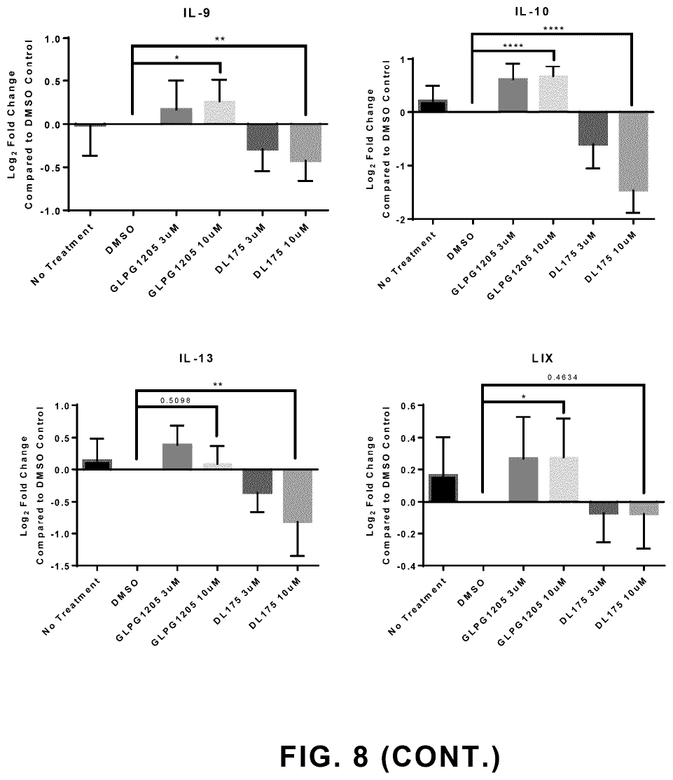

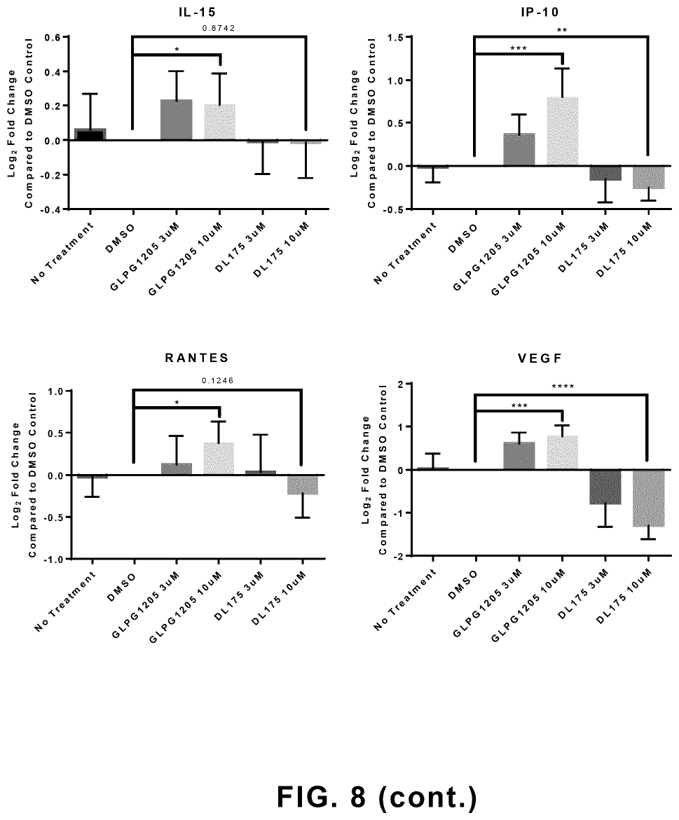

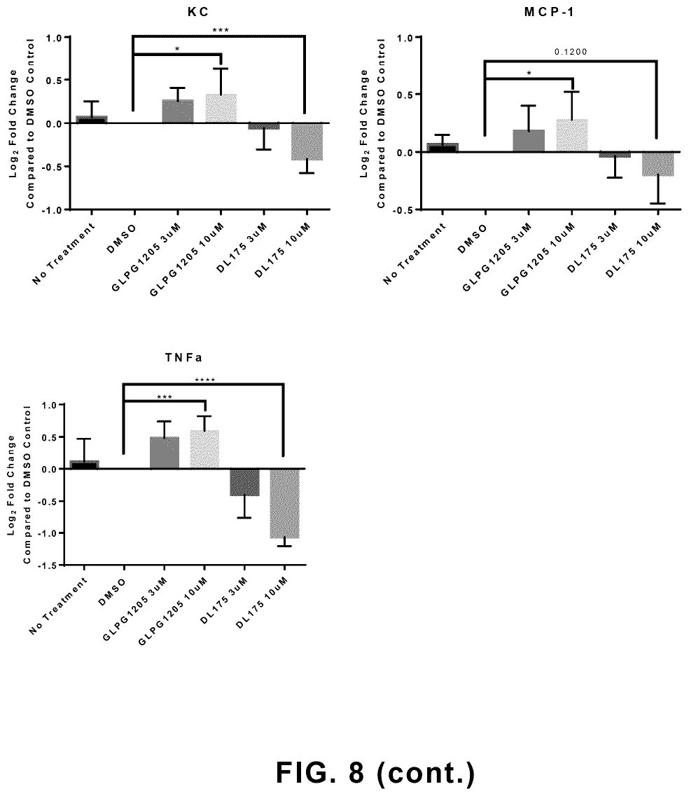

### DETAILED DESCRIPTION OF THE INVENTION

Aspects of the invention are drawn towards compositions and methods for modulating GPR84.

For example, aspects of the invention are drawn towards compositions and methods for increasing the efficacy of anti-tumor immune responses using GPR84 antagonists. Antagonism of the GPR84 receptor promotes the development of increased cytotoxicity in T cells while inducing a decrease in the immunosuppressive function of MDSC. This observation is surprising, for example, because we can modulate the immune response by targeting one receptor that is present in myeloid cells (macrophages, granulocytes, eosinophils, myeloid-derived suppressor cells), T cells (CD8+ killer T cells, CD4+ helper cells and CD4+FoxP3+ regulatory T cells) and Natural Killer cells (NK). Such modulation can cause a beneficial effect. This type of immune modulation can be used to increase the response to a vaccine (such as an anti-cancer vaccine), or to improve the efficacy of an anti-cancer treatment (such as chemotherapy, radiation therapy or immunotherapy).

Also, aspects of the invention are drawn towards compositions and methods for modulating inflammatory diseases using a GPR84 agonist.

By using an agonist for GPR84, immune cells can enter an anti-inflammatory state which could be of benefit in diseases associated with an uncontrolled inflammatory response such as colitis, pancreatitis, hepatitis, myocarditis, rheumatoid arthritis, lupus, early stage of diabetes (pre-diabetic conditions or insulitis) and others. The use of an agonist stimulates the myeloid cells to express arginase 1 and iNOS and causes T cells to decrease their activation as shown by a decrease in the expression of granzyme B and perform (in CD8T cells) and the production of inflammatory cytokines such as IFNg, IL2 and others (in CD4T cells).

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

Unless defined otherwise, all technical and scientific terms used herein have the same meaning as understood by one of ordinary skill in the art to which this disclosure belongs. Although any methods and materials similar or equivalent to those described herein can also be used in the practice or testing of the present disclosure, the advantageous methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and cannot be construed as an admission that the present disclosure is not entitled to antedate such publication by virtue of prior disclosure. Further, the dates of publication provided can be different from the actual publication dates that can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other reasonable order.

Embodiments of the present disclosure will employ, unless otherwise indicated, techniques of medicine, organic chemistry, biochemistry, molecular biology, pharmacology, toxicology, and the like, which are within the skill of the art. Such techniques are explained fully in the literature.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term “subject” or “patient” can refer to any organism to which aspects of the invention can be administered, e.g., for experimental, diagnostic, prophylactic, and/or therapeutic purposes. For example, subjects to which compounds of the disclosure can be administered include animals, such as mammals. Non-limiting examples of mammals include primates, such as humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject. As used herein, “pharmaceutically acceptable derivatives” of a compound can include salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs. As used interchangeably herein, “subject,” “individual,” or “patient,” can refer to a vertebrate, such as a mammal, for example a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

The term “administering” can refer to introducing a substance into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. For example, “parenteral administration” can refer to administration via injection or infusion. Parenteral administration includes, but is not limited to, subcutaneous administration, intravenous administration, and intramuscular administration.

In embodiments, “administering” can also refer to providing a therapeutically effective amount of a formulation or pharmaceutical composition to a subject. The formulation or pharmaceutical compound can be administered alone, but can be administered with other compounds, excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

The term “simultaneous administration” can refer to a first agent and a second agent, when together in the therapeutic combination therapy, are administered either less than about 15 minutes, e.g., less than about 10, 5, or 1 minute. When the first agent and the second agent are administered simultaneously, the first and second treatments can be in the same composition (e.g., a composition comprising both the first and second therapeutic agents) or separately (e.g., the first therapeutic agent is contained in one composition and the second treatment is contained in another composition).

The term “sequential administration” can refer to a first agent and a second agent administered to a subject greater than about 15 minutes apart, such as greater than about 20, 30, 40, 50, 60 minutes, or greater than 60 minutes apart. Either agent can be administered first. For example, the first agent and the second agent can be included in separate compositions, which can be included in the same or different packages or kits.

The term “simultaneous administration” can refer to when the administration of a first agent and a second agent administered to a subject overlap each other.

The terms “co-administration” or the like, as used herein, can refer to the administration of a first active agent and at least one additional active agent to a single subject, and is intended to include treatment regimens in which the compounds and/or agents are administered by the same or different route of administration, in the same or a different dosage form, and at the same or different time.

The term “in combination” can refer to the use of more than one therapies (e.g., one or more prophylactic and/or therapeutic agents). The use of the term “in combination” does not restrict the order in which therapies are administered to a subject with a disease or disorder, or the route of administration.

The term “therapeutically effective amount” can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. A “pharmaceutical composition” can be sterile and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intratracheal, intramuscular, subcutaneous, by stent-eluting devices, catheters-eluting devices, intravascular balloons, inhalational and the like.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used herein can include one and more such excipients, diluents, carriers, and adjuvants.

“Modulating” can refer to regulating or adjusting the degree of activity of a process or the degree of an effect. “Modulating” includes activation, amplification, attenuation, and suppression, for example.

“Treatment” and “treating” can refer to the management and care of a subject for the purpose of combating a condition, disease or disorder, such as an inflammatory response, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

“Preventing” and “prevention” can refer to the prevention of the occurrence of a disease or disorder. In embodiments, the preventative treatment can reduce the recurrence of a disease or disorder.

The phrase “preventing cancer” can refer to prevention of cancer occurrence. In embodiments, the preventative treatment reduces the recurrence of the cancer. In other embodiments, preventative treatment decreases the risk of a patient from developing a cancer or inhibits progression of a pre-cancerous state (e.g., a colon polyp) to actual malignancy.

The term “cancer” can refer to the spectrum of pathological symptoms associated with the initiation or progression, as well as metastasis, of malignant tumors. The term “tumor” can refer to a new growth of tissue in which the multiplication of cells is uncontrolled and progressive. In embodiments, the tumor can be a malignant tumor, one in which the primary tumor has the properties of invasion or metastasis or which shows a greater degree of anaplasia than do benign tumors. Thus, “treatment of cancer” or “treating cancer” can refer to an activity that prevents, alleviates or ameliorates any of the primary phenomena (initiation, progression, metastasis) or secondary symptoms associated with the disease.

Treating cancer can be indicated by, for example, inhibiting or delaying invasiveness of a cancer. “Cancer invasion” can refer to the movement caused by cancer cells in vivo, into or through biological tissue or the like. For example, movements caused by cancer cells into or through barriers formed by special cell-based proteins, such as collagen and Matrigel, and other substances.

The term “anti-cancer” can refer to an action of suppressing the growth of cancer cells or killing cancer cells and an action of suppressing or blocking metastasis of cancer cells in connection with prevention and treatment of cancer. For example, anti-cancer can refer to inhibition of formation, infiltration, metastasis, and growth of cancers.

The term “modulating” can refer to an increase, decrease, or otherwise change an activity. For example, to “modulate the immune system” can refer to stimulating certain responses of the immune system by making it more reactive, for example intervening, via the production of specific cytokines, in development of the cells involved in the immune response. “Modulating the immune system” can also refer to depressing certain responses of the immune system by making it less reactive.

The term “immune system” can refer to the bodily system that protects the body from foreign substances, cells, and tissues by producing the immune response. For example, the immune system can comprise the thymus, spleen, lymph nodes, lymphoid tissue, lymphocytes, and antibodies. As used herein, the term “immune response” can refer to a bodily response to an antigen that occurs when lymphocytes identify the antigenic molecule as foreign and induce the formation of antibodies and lymphocytes.

The term “immune response” can refer to a bodily response to an antigen that occurs when lymphocytes identify the antigenic molecule as foreign and induce the formation of antibodies and lymphocytes.

The terms “inflammation” and “inflammatory reaction” can be used interchangeably. As used herein, the term “inflammation” can refer to a response to cellular injury which can be marked by capillary dilatation, leukocytic infiltration, redness, heat, pain, swelling, and loss of function and serves as a mechanism initiating the elimination of noxious agents and of damaged tissue.

The term “cytokine” can refer to any class of immunoregulatory proteins that are secreted by cells. For example, the cells are cells off the immune system. For example, the cytokine can be produced in the presence of disease or immunization and contribute to immune responses, inflammation, and endothelial cell activation. As used herein, the terms “pro-inflammatory cytokine” and “inflammatory cytokine” can be used interchangeably. In embodiments, pro-inflammatory cytokines can be produced by activated macrophages and are involved in the up-regulation of inflammatory reactions.

The term “cell of the immune system” or “immune cells” can be used interchangeably. For example, cells of the immune system can comprise lymphocytes (T-cells, B-cells, and NK cells), neutrophils, and monocytes/macrophages.

An “autologous cell” can refer to a cell which was derived from the same individual that is being treated by cell therapy.

A “donor cell” can refer to a cell that was derived from an individual other than the individual being treated by cell therapy.

An “allogeneic cell” can refer to a genetically distinct cell.

The term “immunotherapy” can refer to a treatment or prevention of disease that involves stimulation, enhancement, suppression, or desensitization of the immune system. For example, the disease can comprise an autoimmune disorder or cancer.

The term “fatty acid” can refer to a carboxylic acid with an aliphatic chain. For example, the aliphatic chain can be saturated or unsaturated.

The term “antagonist” can refer to a compound or composition that can decrease, block, inhibit, abrogate, or interfere with a biological response by binding to or blocking a cellular constituent. As used herein, the term “agonist” can refer to a compound or composition that interacts with a cellular constituent and elicits an observable response. For example, the cellular constitute comprises a receptor. For example, the receptor can comprise GPR84.

The term “agonist” can refer to any compound that stimulates activity at a receptor or receptors normally stimulated by naturally occurring substances, thus triggering a response.

The term “ligand” can refer to a molecule or agent that interacts with another species. For example, the ligand can comprise an agonist or an antagonist. For example, the species can comprise a receptor. For example, the receptor is can comprise GPR84.

The term “immunotherapeutic agent” can refer to an agent that can be used on or used to modify an immune mechanism or immune response.

The term “chemotherapeutic agent” can refer to any chemical compound useful in the treatment of a neoplastic disease, such as cancer.

The term “in vivo” can refer to an event that takes place in a subject's body.

The term “in vitro” can refer to an event that takes places outside of a subject's body.

The term “ex vivo” can refer to outside a living subject. Examples of ex vivo cell populations include in vitro cell cultures and biological samples such as fluid or tissue samples from humans or animals. Such samples can be obtained by methods well known in the art. Exemplary biological fluid samples include blood, cerebrospinal fluid, urine, saliva. Exemplary tissue samples include tumors and biopsies thereof. In this context, the present compounds can be in numerous applications, both therapeutic and experimental.

The term “genetically engineered cell” can refer to any cell of any organism that is modified, transformed, or manipulated by addition or modification of a gene, a DNA or RNA sequence, or protein or polypeptide. Isolated cells, host cells, and genetically engineered cells include isolated immune cells, such as NK cells and T cells, that contain the DNA or RNA sequences encoding a chimeric receptor or chimeric receptor complex and express the chimeric receptor on the cell surface. Isolated host cells and genetically engineered cells can be used, for example, for enhancing an NK cell activity or a T lymphocyte activity, treatment of cancer, and treatment of infectious diseases.

### Pharmaceutical Compositions

Aspects of the invention are drawn to pharmaceutical compositions comprising a GPR84 antagonist. Non-limiting examples of GPR84 antagonists comprise a compound according to:

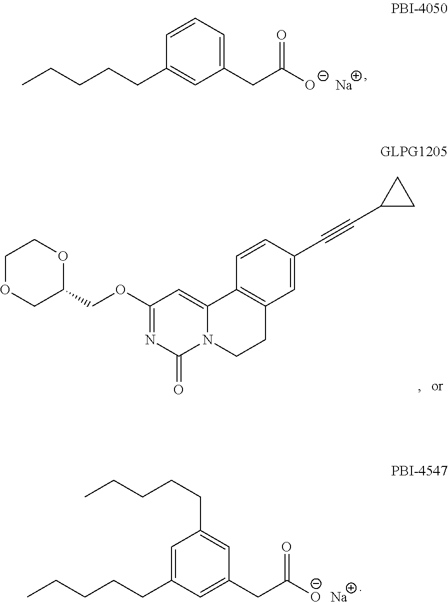 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

In embodiments, the antagonist comprises a compound as described in U.S. Pat. No. 11,098,071.

Aspects of the invention are drawn to pharmaceutical compositions comprising a GPR84 agonist. Non-limiting examples of GPR84 agonists comprise a compound according to:

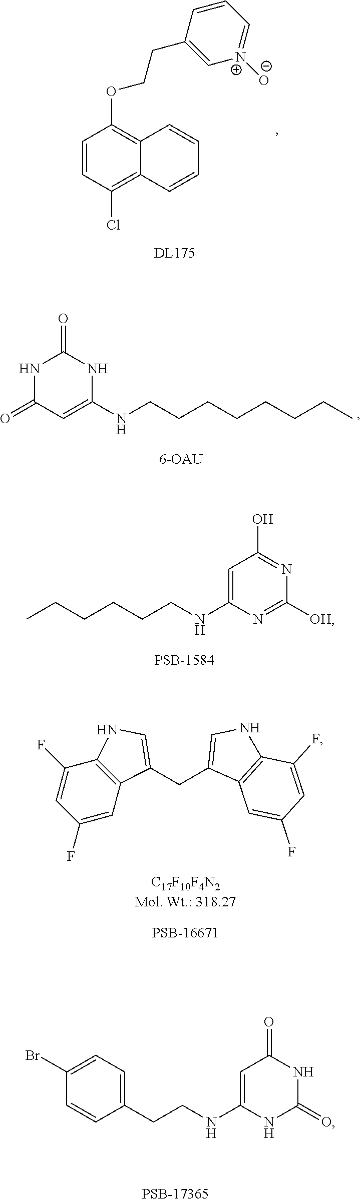

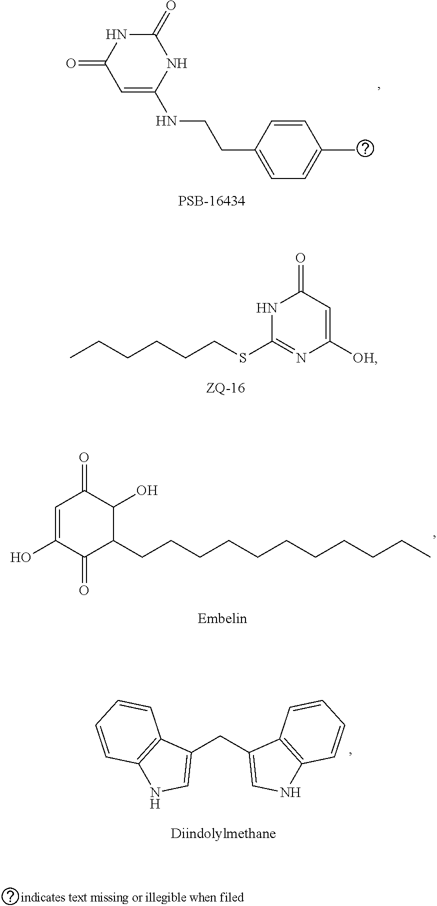

2-HTP, or medium-chain fatty acids

In embodiments, the pharmaceutical composition can comprise at least one additional active agent. Non-limiting examples of the at least one additional active include an anti-cancer agent, an anti-inflammatory agent, a pain reliever, or any combination thereof.

In embodiments, the anti-cancer agent comprises an immunotherapeutic agent. For example, the immunotherapeutic agent can be a genetically engineered cell (e.g., CAR T-cell or CAR NK cell), a monoclonal antibody (e.g., anti-cancer antibody), an adoptive T-cell therapy, an oncolytic virus therapy, a cancer vaccine, an immune system modulator, or any combination thereof. For example, the at least one additional active agent comprises an immunomodulatory agent or an anti-inflammatory agent. For example, the at least one additional anti-inflammatory agent or immunomodulatory treatment comprises an immunosuppressant (e.g., methotrexate), an anti-inflammatory (e.g., sulfasalazine), an antibody, a corticosteroid (e.g., prednisone, prednisolone, methylprednisolone, dexamethasone, or triamcinolone), a non-steroidal anti-inflammatory (e.g., ibuprofen, naproxen, diclofenac, celecoxib, mefenamic acid, etoricoxib, indomethacin), or any combination thereof. For example, the antibody comprises an anti-TNF antibody (e.g., etanercept, infliximab, adalimumab, golimumab, or certolizumab), an anti-IL-5 antibody, an anti-IL4/IL5/IL13 antibody (e.g., dupilumab), an anti-1b antibody, an anti-IL-6 antibody, an anti-IL-17 antibody, or an anti-IL-12/IL-23 antibody (e.g., ustekinumab).

In embodiments, the monoclonal antibody comprises a full-length monoclonal antibody, a fragment antigen binding (Fab) fragment, a single-chain variable fragment (scFv), single-domain antibody (sdAb), a bispecific antibody, or a trispecific antibody.

In embodiments the anti-cancer antibody comprises anti-OX-40, or an anti-checkpoint inhibitor. The term “checkpoint inhibitor” can refer to any molecule, including antibodies and small molecules, that block the immunosuppression pathway induced by one or more checkpoint proteins. For example, the anti-checkpoint inhibitor comprises anti-PD-1 (e.g., pembrolizumab, nivolumab, or cemiplimab), anti-PD-L1, anti-LAG3, or anti-CTLA4 (e.g., ipilimumab).

In embodiments, the anti-cancer agent comprises a chemotherapeutic agent. For example, the chemotherapeutic agent comprises cyclophosphamide, gemcitabine, 5-fluorouracil, docetaxel, doxorubicin, oxaliplatin, mitoxantrone, melphalan, or anthracyclines.

In embodiments, the adoptive T-cell therapy comprises tumor-infiltrating lymphocyte (TIL) therapy, engineered T-cell receptor (TCR) therapy, CAR T-cell therapy, or natural killer (NK) cell therapy.

In embodiments, the anti-cancer agent comprises sipuleucel-T or bacillus Calmette-Guerin.

In embodiments, the oncolytic virus therapy comprises talimogene laherparepvec.

In embodiments, the immune system modulator comprises an interleukin (e.g., IL-2, IL-7, IL-12, or IL-21), an interferon, or an immunomodulators (IMiDs).

Pharmaceutical composition can also be included, or packaged, with other non-toxic compounds, such as pharmaceutically acceptable carriers, excipients, diluents, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, com starch, starch fragments, keratin, colloidal silica, potato starch, urea, dextrans, dextrins, and the like. For example, the pharmaceutically acceptable carriers, excipients, binders, and fillers for use in the practice of the invention are those which render the compounds of the invention amenable to intranasal delivery, oral delivery, parenteral delivery, intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers or silicone, and can be processed internally by the subject without affecting the effectiveness of the composition/formulation packaged and/or delivered therewith.

In embodiments, the pharmaceutical compositions can comprise pharmaceutically acceptable salts. Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl) aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Different forms of the pharmaceutical composition can be calibrated in order to adapt both to different subjects and to the different needs of a single subject. However, the pharmaceutical composition need not counter every cause in every subject. Rather, by countering the necessary causes, the pharmaceutical composition will restore the body to its normal function. Then the body will correct the remaining deficiencies.

For oral preparations, the pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as com starch, potato starch or sodium carboxymethylcellulose; with lubricants, such as talc or magnesium stearate; and if desired, with diluents, buffering agents, moistening agents, preservatives and flavoring agents.

Embodiments of the pharmaceutical composition can be formulated into preparations for injection by dissolving, suspending, or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the composition or pharmaceutical composition can be utilized in aerosol formulation to be administered via inhalation. Embodiments of the composition or pharmaceutical composition can be formulated into pressurized acceptable propellants such as dichiorodifluoromethane, propane, nitrogen and the like.

Unit dosage forms for oral administration, such as syrups, elixirs, and suspensions, can be provided wherein each dosage unit, for example, teaspoonful, tablespoonful, tablet or suppository, contains a predetermined amount of the composition containing one or more compositions. Similarly, unit dosage forms for injection or intravenous administration can comprise the pharmaceutical composition as a solution in sterile water, normal saline or another pharmaceutically acceptable carrier.

### Methods of Treatment

Aspects of the invention are drawn to methods of preventing an inflammatory response in a subject (e.g., inflammation).

Also, aspects of the invention are drawn to methods for preventing, treating, or ameliorating a symptom of an immune system disease or disorder in a subject. For example, the immune system disease or disorder comprises psoriasis, psoriatic arthritis, an auto-immune disease, colitis, asthma, glomerulonephritis, hepatitis, myocarditis, transplant rejection, pre-diabetes, rheumatoid arthritis, lupus, Crohn's disease, graph versus host disease, or a combination thereof.

Further, aspects of the invention are drawn to methods of modulating the immune system of a subject afflicted with or at risk of an immune system disease or disorder. In embodiments, modulating comprises increasing immunosuppressive effects of myeloid-derived-suppressor cells (MDSC), decreasing the pro-inflammatory and cytotoxic effects of inflammatory cells, such as T-cells and NK cells. The term “T cell” can refer to a type of lymphocyte that matures in the thymus. T cells play an important role in cell-mediated immunity and are distinguished from other lymphocytes, such as B cells, by the presence of T cell receptors on the cell surface. T cells can be isolated or obtained from commercial sources. “T cells” are all types of immune cells that express CD3, including helper T cells (CD4+ cells), cytotoxic T cells (CD8+ cells), natural killer T cells, regulatory T cells (Tregs), and immune cells including gamma-delta T cells. “Cytotoxic cells” include CD8+ T cells, natural killer (NK) cells, and neutrophils, which are cells that can mediate a cytotoxic response. The term “NK cells”, also known as natural killer cells, can refer to a type of lymphocyte that is derived from the bone marrow and plays a vital role in the innate immune system. NK cells provide a rapid immune response against virus-infected cells, tumor cells, or other stressed cells, even in the absence of antibodies and major histocompatibility complexes on the cell surface. The term “myeloid-derived suppressor cell” or “MDSC” can refer to a cell of the immune system that modulates the activity of a variety of effector cells and antigen-presenting cells, such as T cells, NK cells, dendritic cells, and macrophages, among others.

In embodiments, the method comprises administering to the subject a therapeutically effective amount of a GPR84 agonist. Non-limiting examples of GPR84 agonists comprise a compound according to:

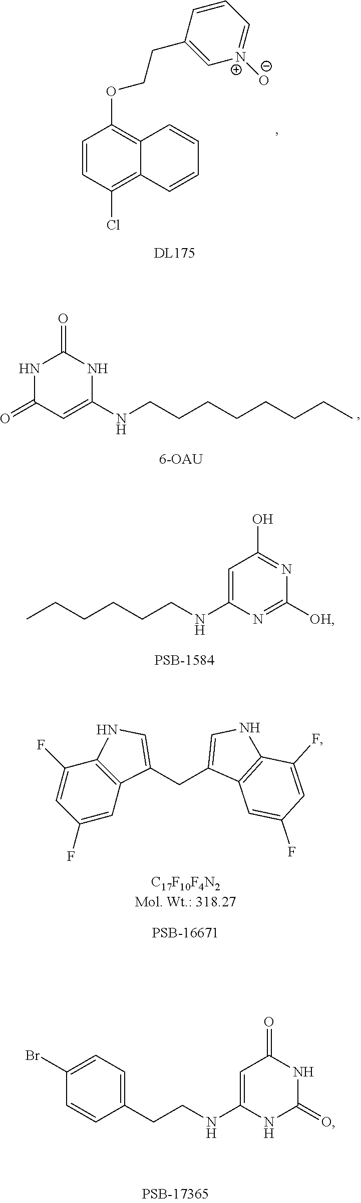

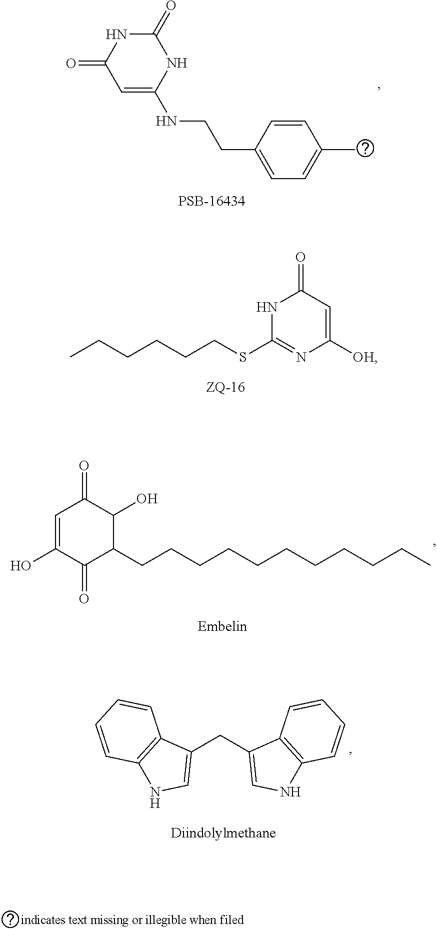

2-HTP, or medium-chain fatty acids

In embodiments, the GPR84 agonist can be provided in a pharmaceutical composition. Non-limiting examples of the GPR84 agonist comprises a compound according to:

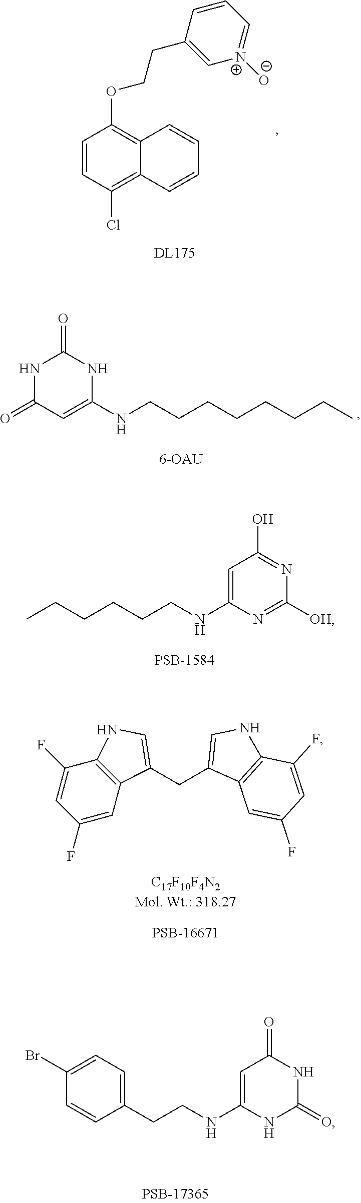

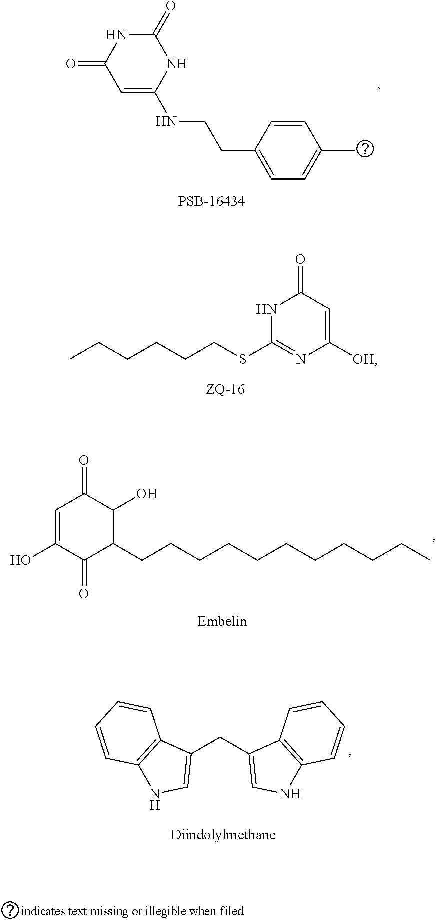

2-HTP, or medium-chain fatty acids

Aspects of the invention are drawn to methods for preventing cancer, preventing the recurrence of cancer, and/or treating cancer in a subject. For example, an embodiment comprises a method of preventing cancer in a subject comprising administering to the subject a therapeutically effective amount of a GPR84 antagonist.

“Cancer” can refer to diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. Also called malignancy.

In embodiments, the cancer can comprise a solid tumor or a liquid cancer. A “solid tumor” can refer to an abnormal mass of tissue that usually does not contain cysts or liquid. A “non-solid tumor”, which can be referred to as a “liquid cancer”, can refer to neoplasia of the hemopoietic system, such as lymphoma, myeloma, and leukemia, or neoplasia without solid formation generally and with spread substantially.

In some embodiment, the solid tumors include but not limited to brain cancer, lung cancer, liver cancer, hepatocellular carcinoma (HCC), esophageal cancer, cholangiocarcinoma, gallbladder carcinoma, stomach cancer, abdominal cancer, gastrointestinal cancer, gastric cancer, pancreatic cancer, renal cell carcinoma, renal cancer, bone cancer, breast cancer, ovarian cancer, uterine cancer, cervical cancer, endometrial cancer, colorectal cancer, colon cancer, rectal cancer, bladder cancer, superficial bladder cancer, prostate cancer, adrenal tumors, squamous cell carcinoma, neuroma, malignant neuroma, myoepithelial carcinoma, synovial sarcoma, rhabdomyosarcoma, gastrointestinal interstitial cell tumor, skin cancer, basal cell carcinoma, malignant melanoma, thyroid cancer, nasopharyngeal carcinoma, hemangioma, epidermoid carcinoma, head and neck cancer, glioma, or Kaposi's sarcoma.

In some embodiments, the non-solid tumors include but not limited to leukemia, acute leukemia, chronic leukemia, chronic myelocytic leukemia, acute myelogenous leukemia, chronic myelogenous leukemia, chronic lymphocytic leukemia, acute lymphoblastic leukemia, T-cell leukemia, hairy cell leukemia, polycythemia, myelodysplastic syndrome, multiple myeloma, lymphadenoma, Hodgkin's lymphoma, and Non-Hodgkin's lymphoma.

In some embodiments, the cancer comprises breast cancer, prostate cancer, colorectal cancer, cervical cancer, lung cancer, lymphoma, leukemia, pancreatic cancer, liver cancer, brain cancer, or skin cancer.

In embodiments, the cancer comprises a cancer that is resistant to immune checkpoint inhibitors. For example, aspects of the invention can sensitize checkpoint resistant tumors to treatment, such as anti-cancer antibodies, by inhibiting suppressive myeloid cells (e.g., MDSC, tumor associated macrophages (TAM) or M2 macrophages) and regulatory-T cells, while at the same time activating T cells (e.g., killer and helper) and NK cells.

In embodiments, the subject afflicted with cancer or at risk of being afflicted with cancer has a familial history of cancer, a chronic inflammatory condition (e.g., colitis, chronic prostatitis), or a genomic mutation that predisposes that subject to the development of cancer. For example, the genomic mutation can be a mutation of BRCA1, BRCA2, MLH1, MSH2, MSH6, PMS2, EPCAM, APC, MUTYH, or OGG1. For example, Lynch syndrome, also known as hereditary non-polyposis colorectal cancer (HNPCC), is the most common cause of hereditary colorectal (colon) cancer. People with Lynch syndrome can get colorectal cancer and other cancers, and at a younger age (before 50), including uterine (endometrial), stomach, liver, kidney, brain, and certain types of skin cancers. Lynch syndrome is due to inherited changes (mutations) in genes that affect DNA mismatch repair, a process that fixes mistakes made when DNA is copied. These genes (MLHL, MSH2, MSH6, PMS2, and EPCAM) normally protect you from getting certain cancers, but some mutations in these genes prevent them from working properly. As another example, the adenomatous polyposis coli (APC) gene is a tumor suppressor gene, and mutations resulting in loss of APC protein function are associated with carcinogenesis. Another familial form of colorectal cancer, MUTYH-associated polyposis (MAP), was first described in families with multiple colorectal adenomas or carcinomas who lacked inherited APC mutations. Also, OGG1 acts together with MYH and MTH1 to identify and remove 8-oxoguanine that has been incorporated into DNA. OGG1 variants have been reported in association with colorectal cancer, alone or in combination with mutations in other genes.

Aspects of the invention are also drawn towards methods of modulating the immune system of a subject. For example, the subject is afflicted with cancer, or at risk of being afflicted with cancer. In embodiments, the method comprises administering to the subject a therapeutically effective amount of a GPR84 antagonist as described herein. In embodiments, the GPR84 antagonist can be provided in a pharmaceutical composition.

In embodiments, modulating comprises increasing the cytotoxicity of T-cells, decreasing immunosuppressive effects of myeloid-derived-suppressor cells, or both.

The term “T cell” can refer to a type of lymphocyte that matures in the thymus. T cells play an important role in cell-mediated immunity and are distinguished from other lymphocytes, such as B cells, by the presence of T cell receptors on the cell surface. T cells can be isolated or obtained from commercial sources. “T cells” are all types of immune cells that express CD3, including helper T cells (CD4+ cells), cytotoxic T cells (CD8+ cells), natural killer T cells, regulatory T cells (Tregs), and immune cells including gamma-delta T cells. “Cytotoxic cells” include CD8+ T cells, natural killer (NK) cells, and neutrophils, which are cells that can mediate a cytotoxic response.

The term “NK cells”, also known as natural killer cells, can refer to a type of lymphocyte that is derived from the bone marrow and plays a vital role in the innate immune system. NK cells provide a rapid immune response against virus-infected cells, tumor cells, or other stressed cells, even in the absence of antibodies and major histocompatibility complexes on the cell surface.

The term “myeloid-derived suppressor cell” or “MDSC” can refer to a cell of the immune system that modulates the activity of a variety of effector cells and antigen-presenting cells, such as T cells, NK cells, dendritic cells, and macrophages, among others.

In embodiments, the GPR84 antagonist can be provided in a pharmaceutical composition. Non-limiting examples of GPR84 antagonists comprise a compound according to.

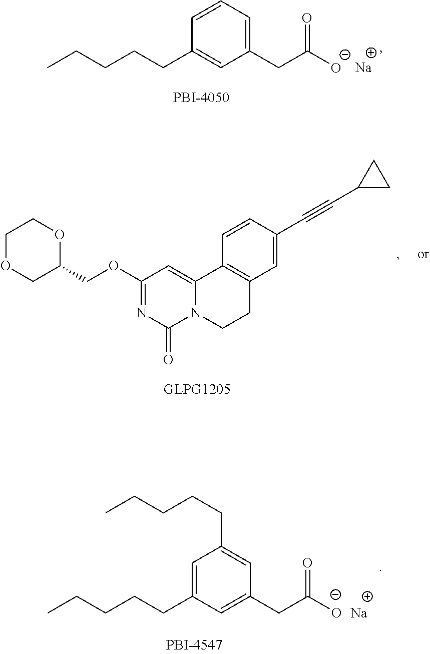 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

In embodiments, the antagonist comprises a compound as described in U.S. Pat. No. 11,098,071

In embodiments, a therapeutically effective amount can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

In embodiments, the therapeutically effective amount comprises less than about 0.1 mg, about 0.1 mg, about 0.5 mg, about 1.0 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 70 mg, about 80 mg, about 90 mg, about 100 mg, about 120 mg, about 135 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 1.0 g, about 1.5 g, about 2.0 g, about 2.5 g, about 5 g, about 10 g, about 25 g, about 50 g, or more than 50 g.

In embodiments, the compound can be administered alone, or can be administered as a pharmaceutical composition together with other compounds, excipients, carriers, diluents, fillers, binders, or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

Embodiments can be administered to a subject in one or more doses. The dose level can vary as a function of the specific composition or pharmaceutical composition administered, the severity of the symptoms and the susceptibility of the subject to side effects. Dosages for a given compound are readily determinable by a variety of means. For example, dosages can be determined by standard clinical techniques. In addition, in vitro or in vivo assays can be employed to help identify optimal dosage ranges. The precise dose to be employed can also depend on the route of administration and can be decided according to the judgment of the practitioner and each patient's circumstances.

In an embodiment, multiple doses of the pharmaceutical composition can be administered. The frequency of administration and the duration of administration of the pharmaceutical composition can vary depending on any of a variety of factors, e.g., patient response, severity of the symptoms, and the like. For example, in an embodiment, the pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. In an embodiment, the pharmaceutical composition can be administered 1 to 4 times a day over a period of time, such as 1 to 10-day time period, or longer than a 10-day period of time.

In embodiments, the GPR84 antagonist can be administered in combination with one or more additional active agents. For example, a first agent (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent (e.g., a prophylactic or therapeutic agent) to a subject with a disease or disorder or a symptom thereof.

In embodiments, the pharmaceutical composition comprising the GPR84 antagonist and the second active agent can be administered sequentially, such as one before the other, or concurrently or simultaneously, such as at about the same time.

### Methods of Manufacturing Cellular Therapies

Aspects of the invention are drawn towards compositions and methods for manufacturing cellular therapies. For example, aspects of the invention comprise methods of culturing a population of cells, and subsequently activating the population of cells (e.g., a T cell, or an NK cell). For example, the method can comprise culturing the population of cells (e.g., T cells or NK cells) in the presence of a GPR84 agonist, whereby the GPR84 agonist maintains the sternness (i.e., non-activated state). Keeping CAR cells in a state of stemness (i.e., not fully mature or active) increases their survival and their therapeutic function once administered to a subject. The use of a GPR84 agonist during the in vitro generation and culture of CAR cells will maintain sternness, while still being able to divide and increase in number.

Aspects of the invention are drawn towards compositions and methods for manufacturing cellular therapies. For example, aspects of the invention comprise methods of activating a cell (e.g., a T cell). To “activate” T cells and Natural killer (“NK”) cells can refer to induce a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

In embodiments, the T cell comprises a CD4 T cell, a CD8 T cell, or a combination thereof.

Embodiments can comprise the step of obtaining a population of cells (e.g., T cells or NK cells) from a subject, and culturing the population of cells in a medium comprising a GPR84 agonist. For example, the population of cells can be maintained in their dormant state by culturing the cells in a medium comprising a GPR84 agonist.

In embodiments, the GPR84 agonist comprises a compound according to:

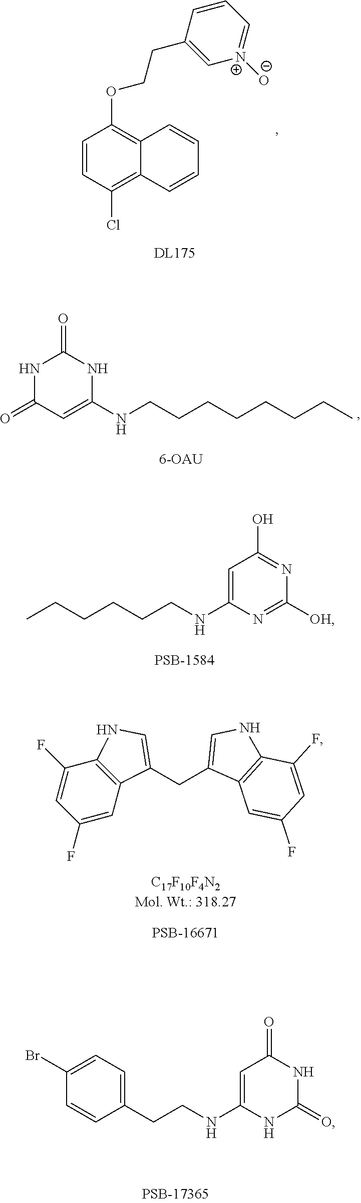

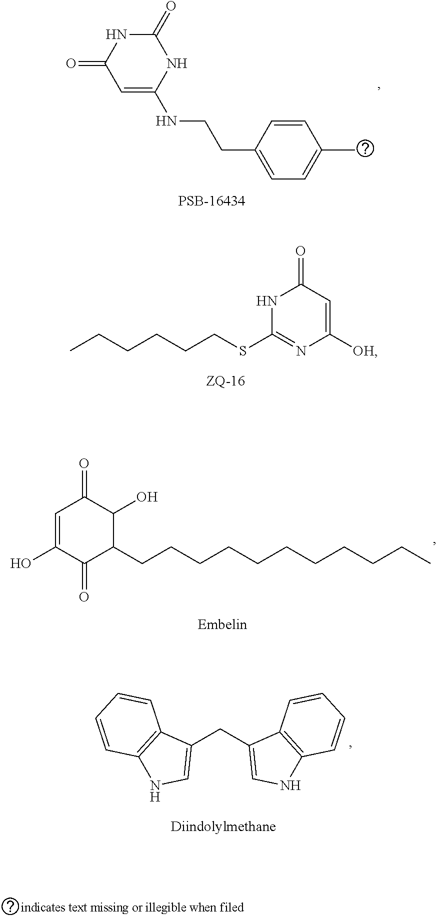

2-HTP, or medium-chain fatty acids

Activating the dormant cells can be accomplished by culturing the cells in a medium comprising a GPR84 antagonist. In embodiments, the GPR84 antagonist comprises a compound according to:

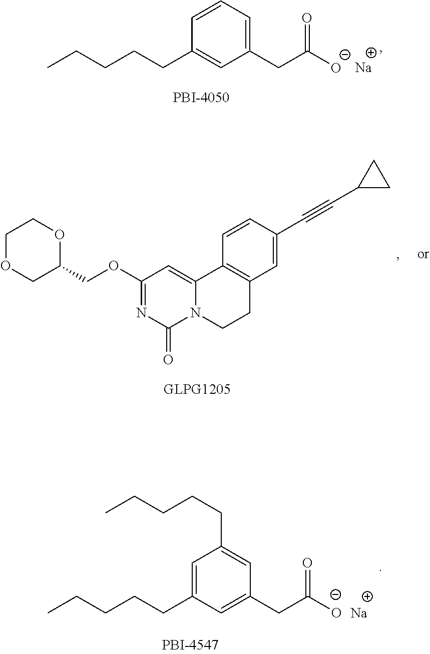 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

In embodiments, the antagonist comprises a compound as described in U.S. Pat. No. 11,098,071.

Activating the cells can be supplemented by additional stimuli, such as anti-CD3, anti-CD28, PSA, Muc-1, MAGE, carcinoembryonic antigen (CEA) or a combination thereof.

For example, the method comprises contacting a cell (e.g., a T cell) with a GPR84 antagonist, wherein the GPR84 antagonist activates the cell. For example, the method can comprise obtaining a population of cells (e.g., T cells) from a subject, and culturing the population of cells in a medium comprising a GPR84 antagonist. For example, the population of cells can be largely dormant prior to culturing in a medium comprising a GPR84 antagonist, which are then activated subsequent to the culturing step.

In embodiments, the method comprises contacting a cell (e.g., a T cell) with a GPR84 antagonist, wherein the GPR84 antagonist activates the cell. For example, the method can comprise obtaining a population of cells (e.g., T cells) from a subject, and culturing the population of cells in a medium comprising a GPR84 antagonist. For example, the population of cells can be largely dormant prior to culturing in a medium comprising a GPR84 antagonist, which are then activated subsequent to the culturing step.

In embodiments, the GPR84 antagonist comprises a compound according to:

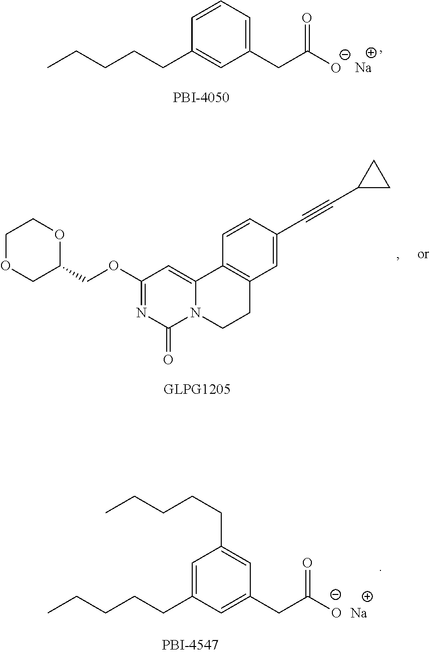 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

Embodiments can further comprise a step of activating the cell (e.g., T cell) with an additional stimuli. For example, the additional stimuli comprises anti-CD3, anti-CD28, PSA, Muc-1, MAGE, carcinoembryonic antigen (CEA) or a combination thereof.

In embodiments, the method is an ex vivo method.

In embodiments, the cell comprises a genetically engineered cell, such as a genetically engineered T cell or a genetically engineered NK cell.

CAR T-cell therapies redirect a patient's T-cells to kill tumor cells by the exogenous expression of a CAR on a T-cell, for example. A CAR can be a membrane spanning fusion protein that links the antigen recognition domain of an antibody to the intracellular signaling domains of the T-cell receptor and co-receptor. A suitable cell can be used, for example, that can secrete an antibody. The antibody “payloads” to be secreted, can be, for example, minibodies, VHH, scFvs, IgG molecules, bispecific fusion molecules, and other antibody fragments. Upon contact or engineering, the cell described herein can then be introduced to a patient in need of a treatment by infusion therapies known to one of skill in the art.

Exemplary CARs and CAR factories useful in aspects of the invention include those disclosed in, for example, PCT/US2015/067225 and PCT/US2019/022272, each of which are hereby incorporated by reference in their entireties. In embodiments, the combination of an agonist and an antagonist of the GPR84 FFAR can be used in the generation of T cells for immunotherapy purposes, in particular CAR-T cells. For example, it has been shown that keeping CAR-T cells in a state of “stemness” (i.e., not fully mature and active), increases their survival and their therapeutic function when transferred in vivo into mice with tumors or in cancer patients. Without being bound by theory, the use of an agonist during the in vitro generation and culture of CAR-T cells can keep them in a state of “stemness”, while still being able to divide and increase in number. Once the CAR-T cells are harvested and infused into a patient with cancer, then one can treat the patient with an antagonist which can increase the maturation and cytotoxic function of the CAR-T cells as they reach the tumor. In addition the antagonist can decrease the immunosuppressive function of the myeloid cells in the tumor, allowing the CAR-T cells to fully execute their anti-tumor effect.

In embodiments, the activated cell (e.g. T cell) can be administered to a subject.

### Kits

Aspects of the invention are directed towards kits, such as kits comprising compositions as described herein. For example, the kit can comprise therapeutic combination compositions described herein.

In one embodiment, the kit includes (a) a container that contains a composition, such as that described herein, and optionally (b) informational material. The informational material can be descriptive, instructional, marketing or other material that relates to the methods described herein and/or the use of the agents for therapeutic benefit.

In an embodiment, the kit includes two or more agents. For example, the kit includes a container comprising a GPR84 antagonist, and a second container comprising a second active agent.

In an embodiment, the kit includes two or more agents. For example, the kit includes a container comprising a GPR84 antagonist, and a second container comprising a second active agent.

In an embodiment, the kit includes two or more agents. For example, the kit includes a containing comprising a GPR84 antagonist, and a second container comprises a GPR84 agonist. In embodiments, the kit further comprises a third container comprising a third active agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the therapeutic combination composition, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

The composition in the kit can include other ingredients, such as a solvent or buffer, a stabilizer, or a preservative. The GPR84 antagonist can be provided in any form, e.g., liquid, dried or lyophilized form, or for example, substantially pure and/or sterile. When the agents are provided in a liquid solution, the liquid solution is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Cells of the immune system use a variety of energy sources to support its functions, including sugars and fats. Fats contain fatty acids and are used in fatty acid oxidation, while sugars are the substrate used in glycolysis. Fatty acids are made of various carbon lengths and degrees of saturation. These variations can change the way and where fatty acids are used in tissue. Currently, there are four families of fatty acids. These are short-chain fatty acids (<5 carbons), medium-chain fatty acids (6-12 carbons), long-chain fatty acids (13-21 carbons) and very-long-chain fatty acids (>22 carbons). Short-chain fatty acids are synthesized by gut intestinal fermentation of indigestible foods. Medium, long, and very-long chains can either be synthesized from cells or obtained through the diet. Recently, FFAs have been more extensively studied as multiple reports have found that they play a role in regulating metabolic disorders. (1, 2)

Fatty acids are not only a component of cellular energy, but also serve as signaling ligands. Free fatty acids (FFA) bind to a family of G-protein coupled receptors (GPCRs), entitled free fatty acid receptors (FFAR) that are found on various cells in the body. Ligation with these GPCRs commits an intracellular signaling cascade which can have global effects on the phenotype, physiology, and action of the cell. Without wishing to be bound by theory, the presence of FFARs can act as an energy sensor and contribute towards regulation of cellular metabolism. FFARs structures restrict the length of the FFA that can bind to them. Currently, there are five main FFARs. These are FFAR1, FFAR2, FFAR3, FFAR4, and GPR84. FFAR1 and FFAR4 only bind long-chain FFA. FFAR2 and FFAR3 can bind to short-chain FFA. The fifth, and least explored FFAR, GPR84, has greatest affinity for medium-chain FFAs. We are interested in the role that the medium chain FFAR GPR84 has on the immune system.(1-4)

GPR84 is a medium-chain fatty acid receptor. It was found through a data mining strategy searching for GPCRs. [5] Still defined as an orphan receptor, its ligand, MCFAs, was not discovered until years later.[6] Further examination found that GPR84 is Gi/o GPCR, and interaction with its ligand leads to an intracellular reduction of cyclic adenosine monophosphate (cAMP). GPR84 is distributed among various tissues in the body, such as adipocytes, but also, GPR84 is found to have high expression in immune cells. In studies of adipogenesis, GPR84 was upregulated in human adipocytes stimulated with inflammatory cytokines and mice fed with high-fat diet. (7) In the immune tissue, GPR84 is expressed in monocytes, macrophages, granulocytes, T cells, and B cells. (8, 9) These studies found that immune cells exposed to a pro-inflammatory stimulus induces high expression of GPR84.[10] Other metabolically contributing factors which increase GPR84 expression in macrophages include hyperglycemia hypercholesterolemia, and oxidized LDL.[6, 11] Studies examining the role of GPR84 in T cells found that GPR84 deficient mice increased IL-4, IL-5 and IL-13, but had no effect on IL-2 or IFN-gamma production.[9]

Without being bound by theory, GPR84 acted as a feed-forward mechanism during inflammation. In this mechanism, a pro-inflammatory stimulus increases the expression of GPR84, and further signaling through GPR84 exacerbates this inflammatory response. This pathway was shown in one study where immune cells exposed to LPS induced GPR84 and that supplementing with MCFA increased the production of IL-12p40.[6] These feed-forward pro-inflammatory findings developed a therapeutic void and lead to the development of GPR84 agonists.

The synthetic agonist, 6-OAU, was found the increase chemotaxis of human polymorphonuclear leukocytes and macrophages and increase proinflammatory cytokine production.[12] In a separate study, GPR84 agonism under inflammatory conditions elevated pro-inflammatory cytokines and increased bacterial phagocytosis and this elevated response was abrogated in GPR84 deficient mice or in macrophages treated with a GPR84 antagonist.[11] A separate natural GPR84 agonist, Embelin, was also examined. Here, studies found that treatment with Embelin lead to neutrophil chemotaxis and primed these cells for oxidative burst and that loss of GPR84 abrogated the pro-inflammatory cytokine secretion.[13]A synthesized GPR84 agonist, DL175, was also found to promote phagocytosis of in human myeloid cells. [14]

The agonist used in vivo was conducted in nude mice, which lack the immune system and cannot provide any evidence that GPR84 agonists modulate the immune system. These controlled systems have raised skepticism that in these studies the agonists used can or cannot be acting through the GPR84 receptor at all. [19-22]

These findings regarding myeloid chemotaxis and metabolic regulation lead researchers to examine GPR84's anti-inflammatory and metabolic regulation. In studies using antagonists, a goal was to prevent chemotaxis and to elicit and anti-inflammatory response. The first antagonist created, PBl-4050, was found to be both an agonist of GPR40 and an antagonist of GPR84. PBl-4050 was found to affect macrophages, fibroblasts, myofibroblasts, and epithelial cells. Antagonism reduced fibrotic capacity of these cells while also dampening macrophage activation and secretion of pro-inflammatory and pro-fibrotic molecules. In addition, macrophage recruitment was prevented, which are contributors to fibrotic disease.[15] The use of GLPG1205, a GPR84 specific receptor antagonist was used in the reduction of inflammation and fibrosis. This study also found that antagonism of GPR84 led reductions in neutrophils, monocytes, and macrophage migration. When examined in three separate models of non-alcoholic steato-hepatitis (NASH), there was a significant decrease in macrophage accumulation as well as reductions in both inflammation and fibrosis.[16] Both studies showed similar outcomes and indicated that the use of antagonisms as an anti-inflammatory and anti-fibrotic drug can be therapeutic.

A study examined in depth the role of GPR84 in metabolic deregulation. Supplementing previous GPR84 antagonist literature, this study examined PBI-4547, a GPR40 and PPAR gamma agonist with some GPR84 antagonist, was studied in Non-alcoholic fatty liver disease (NAFLD). A model of diet-induced obesity was used and antagonism of GPR84 with PBI-4547 improved metabolic dysregulation through reductions in hyperglycemia and hypertriglyceridemia. When examining the effects in GPR84 deficient mice, this ability to regulate the metabolism was absent indicating GPR84 receptor can play a role. The mechanistic ability to regulate metabolism was due to an ability to modulate glycolysis and fatty acid oxidation. Researchers found increases in glucose transporters and upregulations of enzymes which leading to improved glycolysis. In addition, increases in fatty acid oxidation led to improve metabolism of fatty acid oxidation. Both metabolic adjustments reduced overall metabolic dysregulation and returned the system back to physiological homeostasis.[17] Supplementing these findings, a separate study found that the use of a separate GPR84 modulator, PBI-4050, also improved glycemic control, indicating that GPR84 can have a role in metabolic regulation. [18]

Our results are different in that they indicate that GPR84 acts as regulator of functions in T lymphocytes and myeloid cells (macrophages, myeloid derived suppressor cells and others) that will enhance the antitumor effect of immunotherapy. Most tumors are poorly immunogenic, i.e., the protein or markers they express are not strongly antigenic and therefore the immune response generated by T cells can be weak. Even when a strong response is generated the tumor microenvironment (TME) creates a major barrier for T cells to function appropriately. The TME impairs the normal protective immune responses by decreasing the ability of T cells to kill tumor cells and by increasing the presence of myeloid cells able to suppress T cell function (myeloid-derived suppressor cells or M2 macrophages). T cells kill tumor cells through the production of proteins including Granzyme B and perform, which when released in the proximity of a tumor cell, cause the formation of pores or holes in the membrane of the tumor cell and effectively kill it. In contrast myeloid-derived suppressor cells or M2 macrophages produce Arginase 1, nitric oxide or reactive oxygen and nitrogen species, which impair the function of T cells. This allows tumor cells to escape the innate (Natural Killer and macrophages) and adaptive (T cells and B cells) responses and continue to grow and metastasize. The mechanisms by which tumors impair the immune response are multiple and include the expression of checkpoint inhibitors such as PD-1, PD-L1, and CTLA-4, the depletion of amino-acids such as arginine by Arginase 1, the killing of T cells by the production of reactive oxygen species or reactive nitrogen species (Nitric Oxide), and changes in the metabolic microenvironment.

Our results indicate that the use of antagonists of GPR84 dramatically increase the expression of perform and granzyme B in T cells (both CD8+ cytotoxic T cells and CD4+ helper T cells), making them more cytotoxic against tumor cells. At the same time, the use of GPR84 antagonists decreases the expression of Arginase 1 and Nitric oxide synthase (NOS2) making MDSC less immunosuppressive. A surprising result described herein is a single agent, an antagonist to GPR84 FFAR, simultaneously increases the ability of T cells to kill tumors while decreasing the ability of MDSC to suppress the protective T cell response. Since T cells and MDSC are found to infiltrate tumor tissues, without wishing to be bound by theory, the use of a GPR84 antagonist can reverse the immunosuppressive tumor microenvironment and potentiate the therapeutic anti-tumor immune response. Non-limiting, exemplary uses of these observations comprise:

1. The use of GPR84 antagonists in the prevention of cancer: GPR84 antagonists can be given to patients with a high risk of developing cancer including (but not limited to) patients with a strong family history of cancer (breast, prostate, colorectal or other), patients with a chronic inflammatory condition such as colitis, chronic prostatitis, and patients with genomic mutations that place them at high risk for developing cancer.

2. The use of GPR84 antagonists to prevent recurrence of cancer: GPR84 can be given to patients who have undergone chemotherapy or the resection of a primary tumor such as colorectal or lung cancer and have a high risk of having a recurrence.

3. The use of GPR84 antagonists in the treatment of cancer: Since the immune system can initiate a natural response against certain tumor for example melanoma, or cervical cancer, then GPR84 antagonists can be used to enhance the function of the cytotoxic T cells and decrease the suppressive functions of myeloid cells.

4. The use of GPR84 antagonists in the treatment of cancer in conjunction with immunotherapy, chemotherapy, or radiation therapy.

5. The use of GPR84 antagonists in the generation of chimeric antigen receptor-T cells (CAR-T) for their use in adoptive cellular therapy.

Our data indicates the use of GPR84 antagonists can be effective in enhancing the treatment of cancer.

The use of GPR84 antagonists as an anti-tumor immune response can have broad implications in the treatment of cancer. Without wishing to be bound by theory, the use of GPR84 antagonists can enhance the efficacy of anti-tumor immune cells. This therapy can be useful in any cancer where an insufficient anti-tumor immune response occurs, and in situations where GPR84 antagonism elicits a strong anti-tumor response, antagonism can be used as a single agent anti-cancer agent. In addition, in situations where the goal is to enhance a muted anti-tumor response, due to the strong safety profile of GPR84 antagonists, combination therapies can be warranted. Some examples include combination therapies with immune checkpoint inhibitors and enhancing CAR-T cell therapy. The use a GPR84 antagonist to boost the immune activity can also switch immune cells which have entered a ‘dormant’ or ‘immunosuppressive’ state to an activated proinflammatory immune cell. In these situations, GPR84 antagonism can be beneficial in tumors which have high degrees of senescent anti-tumor immune cells or tumors which contain high amounts of immunosuppressive cells such as the myeloid derived suppressor cell.

Technologies used to enhance the immune response to cancer use cytokines such as interleukin 2, interferon gamma or interleukin 12. Even though these are naturally produced by the immune system, their use in the clinical setting requires the infusion of large doses of these cytokines which results in high levels of toxicity. Furthermore, these cytokines have an impact on T cells or NK cells or macrophages, but not on all the cells at the same time. Other forms of increasing the immune response to cancer use adjuvants such as Montanide ISA-51 or ISA-720, carboxymethylcellulose (Poly-lCLC), monophosphoryl lipid A (MPLA), imiquimod, CpG oligodeoxynucleotide (CpG ODN) and others, which are used by mixing them with vaccines. These can increase the presentation of the vaccine antigen to T cells by dendritic cells, but have little impact themselves on the tumor microenvironment (TME).

Approaches have been used to target MDSC. Among these is the use of retinoic acid which promotes the maturation of MDSC into granulocytes, or arginase inhibitors which block the function of arginase and thus prevent the depletion of the amino-acid arginine.

The use of FFARs in metabolism has been studied. Dietary supplementation of omega-3-fatty acids has been studied as a way of decreasing inflammation, but these approaches have remained in the category of dietary supplements rather than in the form of therapies. Some recent studies have begun which explore the modulation of the immune system through FFARs.[23] However, the focus of these therapies have been on FFAR1-4, while limited studies have begun on the less studied GPR84 FFAR. Most of this literature points to the effect that agonists of the GPR84 contribute to a pro-inflammatory response. In contrast, our results indicate instead that antagonism of the GPR84 receptor promotes the development of increased cytotoxicity in T cells while inducing a decrease in the immunosuppressive function of MDSC. Therefore, this unexpected and simultaneous function has been undescribed and therefore represents a different approach to cancer therapy. Some differences are that it is a report on the less-studied GPR84 FFAR as a contributor in modulating the immune response in cancer. It also indicates that the results are obtained by using antagonists rather than agonists of the GPR84 FFAR. Non-limiting, exemplary advantages of this mechanism are that antagonism of GPR84 can act as both a stand-alone treatment in the therapy of certain cancers and as a combination treatment with existing therapies such as approved immunotherapies and CAR-T cell therapies. Without wishing to be bound by theory, antagonism of GPR84 can modulate the immune system in cancer, allowing us to apply this therapy in any cancers which are susceptible to the anti-cancer properties of the immune system.

Various GPR84 antagonists which have been brought to clinical trials. PBl-4050 and GLPG1205, the currently two known GPR84 antagonists have both successfully been tested in phase II clinical trials and have strong safety profiles.

In regard to the safety profile, PBI-4050 is being tested as a treatment for idiopathic pulmonary fibrosis. Results found that GPR84 antagonist is well tolerated both as a single agent and in combination with nintedanib or pirfenidone in a phase II clinical trial. Results found that the ability to increase forced vital capacity was unsuccessful in patients both as a single agent and in combination with nintedanib. More so, without wishing to be bound by theory, combination therapy of PBI-4050 with pirfenidone worsened forced vital capacity (FVC), which indicates acceleration of disease. Arguments for a reduction in FVC and acceleration in disease can be due to a drug-drug interaction between PBI-4050 and pirfenidone. [25, 26] When further examining the effects of PBI-4050 on inflammatory and fibrotic biomarkers, results found that patients treated with PBI-4050 had significant increase in IL-9, IL-7, MIP-Ibeta, with near-significant increases in IL-IRa, IL-13, and G-CSF.[27] While the argument of reduction of inflammation occurs, some of these markers such as MIP-Ibeta and G-CSF are markers of inflammation. Such inflammatory increases can support the position whereby an unknown proinflammatory pathway can be stimulated with GPR84 antagonism.[27]

PBI-4050 has also been examined as a treatment in type II diabetic patients with metabolic syndrome. A phase II study found that treatment with PBl-4050 can reduce HbAlc in type II diabetic patients with metabolic syndrome.

The other GPR84 antagonist that has been used in clinical trials is GLPG1205. GLPG1205 is a negative allosteric modulator of GPR84 that has progressed through phase II clinical trials. Initial phase I studies found that GLPG1205 occupied the human GPR84 receptor, was well tolerated and had a terminal half-life of 30.1 to 140 hours. [29] Additionally, when studying the interaction of GLPG1205 with the cytochrome family of enzymes and were found to have strong safety profiles ensuring that this drugs administration can be used concomitantly with other therapies. [30]

GLPG1205 has also been studied in the treatment of interstitial pulmonary fibrosis. Results provided a non-significant reduction in fibrosis in all groups that were supplemented with GLPG1205 over the standard of care, which warranted further studies to determine if GLPG1205 does have an effect in this disease.

GLPG1205 use was also explored as a treatment for ulcerative colitis. Results from this study found that GLPG1205 was unable to improve the inflammatory response. In fact, GLPG1205 was found to lead to worsening of colitis in certain patients in the treatment study which led to the drug's discontinuation.[31] Without wishing to be bound by theory, the antagonism of the GPR84 receptor can lead to a pro-inflammatory response.

### REFERENCES CITED IN THIS EXAMPLE

1. Kimura, I., et al., Free Fatty Acid Receptors in Health and Disease. Physiol Rev, 2020. 100(1): p. 171-210.
2. Hara, T., et al., Role of free fatty acid receptors in the regulation of energy metabolism. Biochim Biophys Acta, 2014. 1841(9): p. 1292-300.
3. Alvarez-Curto, E. and G. Milligan, Metabolism meets immunity: The role of free fatty acid receptors in the immune system. Biochem Pharmacol, 2016. 114: p. 3-13.
4. Hara, T., et al., Free fatty acid receptors and their role in regulation of energy metabolism. Rev Physiol Biochem Pharmacol, 2013. 164: p. 77-116.
5. Wittenberger, T., H. C. Schaller, and S. Hellebrand, An expressed sequence tag (EST) data mining strategy succeeding in the discovery of new G-protein coupled receptors. J Mol Biol, 2001. 307(3): p. 799-813.
6. Wang, J., et al., Medium-chain fatty acids as ligands for orphan G protein-coupled receptor GPR84. J Biol Chem, 2006. 281(45): p. 34457-64.
7. Nagasaki, H., et al., Inflammatory changes in adipose tissue enhance expression of GPR84, a medium-chain fatty acid receptor: TNFα enhances GPR84 expression in adipocytes. FEBS Lett, 2012. 586(4): p. 368-72.
8. Yousefi, S., et al., Cloning and expression analysis of a novel G-protein-coupled receptor selectively expressed on granulocytes. J Leukoc Biol, 2001. 69(6): p. 1045-52.
9. Venkataraman, C. and F. Kuo, The G-protein coupled receptor, GPR84 regulates IL-4 production by T lymphocytes in response to CD3 crosslinking. Immunol Lett, 2005. 101(2): p. 144-53.
10. Pillaiyar, T., et al., 6-(Ar)Alkylamino-Substituted Uracil Derivatives: Lipid Mimetics with Potent Activity at the Orphan G Protein-Coupled Receptor 84 (GPR84). ACS Omega, 2018. 3(3): p. 3365-3383.
11. Recio, C., et al., Activation of the Immune-Metabolic Receptor GPR84 Enhances Inflammation and Phagocytosis in Macrophages. Front Immunol, 2018. 9: p. 1419.
12. Suzuki, M., et al., Medium-chain fatty acid-sensing receptor, GPR84, is a proinflammatory receptor. J Biol Chem, 2013. 288(15): p. 10684-91.
13. Gaidarov, I., et al., Embelin and its derivatives unravel the signaling, proinflammatory and antiatherogenic properties of GPR84 receptor. Pharmacol Res, 2018. 131: p. 185-198.
14. Lucy, D., et al., A Biased Agonist at Immunometabolic Receptor GPR84 Causes Distinct Functional Effects in Macrophages. ACS Chem Biol, 2019. 14(9): p. 2055-2064.
15. Gagnon, L., et al., A Newly Discovered Antifibrotic Pathway Regulated by Two Fatty Acid Receptors: GPR40 and GPR84. Am J Pathol, 2018. 188(5): p. 1132-1148.
16. Puengel, T., et al., The Medium-Chain Fatty Acid Receptor GPR84 Mediates Myeloid Cell Infiltration Promoting Steatohepatitis and Fibrosis. J Clin Med, 2020. 9(4).
17. Simard, J. C., et al., Fatty acid mimetic PBI-4547 restores metabolic homeostasis via GPR84 in mice with non-alcoholic fatty liver disease. Sci Rep, 2020. 10(1): p. 12778.
18. Li, Y., et al., Fatty acid receptor modulator PBI-4050 inhibits kidney fibrosis and improves glycemic control. JCI Insight, 2018. 3(10).
19. Firestone, G. L. and L. F. Bjeldanes, Indole-3-carbinol and 3-3′-diindolylmethane antiproliferative signaling pathways control cell-cycle gene transcription in human breast cancer cells by regulating promoter-Sp1 transcription factor interactions. J Nutr, 2003. 133(7 Suppl): p. 2448s-2455s.
20. Kiselev, V. I., et al., Preclinical antitumor activity of the diindolylmethane formulation in xenograft mouse model of prostate cancer. Exp Oncol, 2014. 36(2): p. 90-3.
21. Nachshon-Kedmi, M., et al., Indole-3-carbinol and 3,3′-diindolylmethane induce apoptosis in human prostate cancer cells. Food Chem Toxicol, 2003. 41(6): p. 745-52.
22. Marsango, S., et al., Therapeutic validation of an orphan G protein-coupled receptor: The case of GPR84. Br J Pharmacol, 2020.
23. Bartoszek, A., et al., Free Fatty Acid Receptors as New Potential Targets in Colorectal Cancer. Curr Drug Targets, 2020. 21(14): p. 1397-1404.
24. Mancini, S. J., et al., On-target and off-target effects of novel orthosteric and allosteric activators of GPR84. Sci Rep, 2019. 9(1): p. 1861.
25. Khalil, N., et al., Phase 2 clinical trial of PBI-4050 in patients with idiopathic pulmonary fibrosis. European Respiratory Journal, 2019. 53(3).
26. Parker, J., et al., PBI-4050 is safe and well tolerated and shows evidence of benefit in idiopathic pulmonary fibrosis, in B14. CLINICAL TRIALS ACROSS PULMONARY DISEASE. 2017, American Thoracic Society. p. A7606-A7606.
27. Gagnon, L., et al., Evaluation of the Effect of PBI-4050 Alone or in Combination with Pirfenidone or Nintedanib on Blood Biomarkers Linked to the Fibrotic Process in IPF Patients, in A93. ILD: CLINICAL TRIALS. 2018, American Thoracic Society. p. A2439-A2439.
28. Laurin, P., et al., PBI-4050 Reduces Cardiovascular Biomarkers in Type II Diabetic Patients With Metabolic Syndrome. Circulation, 2016. 134(suppl_1): p. A19469-A19469.
29. Timmis, H., et al., GLPG1205, a GPR84 modulator: safety, pharmacokinetics, and pharmacodynamics in healthy subjects. Clinical Pharmacology in Drug Development, 2021.
30. Desrivot, J., et al., Effect of GLPG1205, a GPR84 Modulator, on CYP2C9, CYP2C19, and CYP1A2 Enzymes: In Vitro and Phase 1 Studies. Clinical Pharmacology in Drug Development, 2021.
31. Vermeire, S., et al., P610 efficacy and safety of GLPG1205, a GPR84 antagonist, in ulcerative colitis: multi-Centre proof-of-concept study. Journal of Crohn's and Colitis, 2017. 11(suppl_1): p. S390-S391.

### Example 2

Cells of the immune system use a variety of energy sources to support its functions, including sugars and fats. Fats contain fatty acids and are used in fatty acid oxidation, while sugars are the substrate used in glycolysis. Fatty acids are made of various carbon lengths and degrees of saturation. These variations can change the way and where fatty acids are used in tissue. Currently, there are four families of fatty acids. These are short-chain fatty acids (<5 carbons), medium-chain fatty acids (6-12 carbons), long-chain fatty acids (13-21 carbons) and very-long-chain fatty acids (>22 carbons). Short-chain fatty acids are synthesized by gut intestinal fermentation of indigestible foods. Medium, long, and very-long chains can either be synthesized from cells or obtained through the diet.

Fatty acids are not only a component of cellular energy, but also serve as signaling ligands. Free fatty acids (FFA) bind to a family of G-protein coupled receptors (GPCRs), entitled free fatty acid receptors (FFAR) that are found on various cells in the body. Ligation with these GPCRs commits an intracellular signaling cascade which can have global effects on the phenotype, physiology, and action of the cell. Without wishing to be bound by theory, the presence of FFARs can act as an energy sensor and contribute towards regulation of cellular metabolism. FFARs structures restrict the length of the FFA that can bind to them. Currently, there are five main FFARs. These are FFAR1, FFAR2, FFAR3, FFAR4, and GPR84. FFAR1 and FFAR4 only bind long-chain FFA. FFAR2 and FFAR3 can bind to short-chain FFA. The fifth, and least explored FFAR, GPR84, has greatest affinity for medium-chain FFAs.

Aspects of the invention are drawn towards treating or preventing GPR84 responsive conditions. In embodiments, antagonism of the GPR84 receptor promotes the development of increased cytotoxicity in T cells while inducing a decrease in the immunosuppressive function of myeloid-derived-suppressor cells (MDSC).

Aspects of the invention are directed towards modulating the immune system by administering a GPR84 antagonist. As used herein, the term “modulating the immune system” can refer to increasing T-cells and decreasing immunosuppressive effects of myeloid-derived-suppressor cells, or both. Aspects of the invention are drawn towards administering a GPR84 antagonist to promote the development of increased cytotoxicity in T cells while inducing a decrease in the immunosuppressive function of MDSC.

Antagonists of GPR84 dramatically increases the expression of perform and granzyme B in T cells (both CD8+ cytotoxic T cells and CD4+ helper T cells), making them more cytotoxic against tumor cells. At the same time, the use of GPR84 antagonists decreases the expression of Arginase 1 and Nitric oxide synthase (NOS2) making MDSC less immunosuppressive. A surprising result described herein is a single agent, an antagonist to GPR84 FFAR, simultaneously increases the ability of T cells to kill tumors while decreasing the ability of MDSC to suppress the protective T cell response. Since T cells and MDSC are found to infiltrate tumor tissues, the use of a GPR84 antagonist can reverse the immunosuppressive tumor microenvironment and potentiate the therapeutic anti-tumor immune response.

### Example 3

Immune cells use a variety of metabolites to produce energy needed to carry out their respective functions. Research explores the effects that different energy sources can have on the function of immune cells. Many immune cells will use metabolite sensing receptors to detect different energy sources found in the extracellular environment. This mechanism is useful because it allows these cells to optimize their energy usage. Herein, the medium chain free fatty acid receptor (FFAR), GPR84 is described. Research has begun describing how FFARs can play a role in metabolic diseases. However, it remains to be explored how these FFARs can directly work in the role of both cancer and inflammation.

Described herein is modulation of the immune system using antagonists to the GPR84 FFAR. This observation is surprising, including but not limited to, that by targeting one receptor that is present in myeloid cells (macrophages, granulocytes, eosinophils, myeloid-derived suppressor cells), T cells (CD8+ killer T cells, CD4+ helper cells and CD4+FoxP3+ regulatory T cells) and Natural Killer cells (NK) we can regulate the immune response to cause a beneficial therapeutic effect.

A GPR84 antagonist increases the activity and inflammatory phenotype. This can be useful in diseases that require a highly activated and protective T cell response such as cancer, infectious diseases, improving the efficacy of vaccines or cancer immunotherapy, and others. This is caused by an increase in the use of glucose by T cells and the increased expression of granzyme B and perform (in CD8 T cells), an increase in inflammatory helper cytokines IL2 and IFNγ in CD4 T cells and a decrease in the expression of arginase 1 and iNOS in myeloid cells.

Without wishing to be bound by theory, a mechanism to modulate the immune response through GPR84 is due to control on the metabolic state of the immune cells. Immune cells that are more glycolytic have a more inflammatory phenotype while immune cells that use fatty acid oxidation are found to be more anti-inflammatory. The use of a GPR84 antagonists can lead cells to rely greater on glycolysis and therefore obtain a more pro-inflammatory phenotype. In contrast, GPR84 agonists can lead to less of a reliance on glycolysis and therefore present with a more anti-inflammatory phenotype. Our results indicate that this reprogramming can occur in both anti-cancer T cells as well as pro-cancer myeloid derived suppressor cells (MDSCs). In T cells, use of an agonist was found to reduce glycolysis and prevent T cells activation, proliferation and maturation. Use of an antagonist lead to an opposite effect, stimulating glycolysis and exhibiting a stronger cytotoxic phenotype. In the fatty acid oxidation reliant MDSC, agonism enhanced immunosuppressive phenotypes while antagonism reduced these phenotypes and immunosuppressive capacity. These results indicate that reprogramming immune cell metabolism is an effective strategy in enhancing anti-tumor capacity and provides a new approach in the treatment of diseases.

### Example 4

1.5×10⁵ MC38 colon adenocarcinoma were subcutaneously inoculated into 7-8 week old C57BL/6 mice. Mice were randomized into four treatment groups: vehicle control, anti-CTLA4, 30 mg GLPG1205, or anti-CTLA4+30 mg GLPG1205. Checkpoint inhibitor therapy was administered on day 5, 8, and 11. First dose was 200 μg anti-CTLA4, and subsequent doses were 100 μg anti-CTLA4, administered by intraperitoneal injection. 30 mg GLPG1205 was suspended in 0.5% methylcellulose and administered twice daily by oral gavage. Tumor size and mouse weight were measured three times per week for 21 days.

Other checkpoint inhibitors to use include anti-PD-1 and anti-PD-L1. Other cancers to use include B16 melanoma, 3LL lung carcinoma, and mWnt triple-negative breast cancer.

### Example 5

Wojciechowicz, Megan L., and Avi Ma'ayan. “GPR84: an immune response dial?.” Nature reviews. Drug discovery 6 (2020): 374 for example, shows a schematic of the impact of GPR84 can have on modulating the immune system. However, it describes a dial in the opposite direction of our observations. They indicate that using a GPR84 agonist will act as a pro-inflammatory signal and will be useful in the treatment of diseases that require an active immune response, such as cancer. In contrast using an antagonist will lead to an anti-inflammatory response and can be useful in the treatment of certain inflammatory diseases, such as ulcerative colitis, Crohn's disease, and arthritis. This is opposite our observations where using an agonist stimulates anti-inflammatory responses and using and antagonist stimulates an inflammatory protective response.

### Example 6—Medium Chain Fatty Acid Receptor GPR84 Modulates Anti-Tumor Immunity Through Metabolic Reprogramming

### Materials and Methods

### Cell Lines and Culture Media Preparation

The immortalized cell lines B16-F10 (ATCC CRL-6475), EL-4 (ATCC #TIB-39), E.G7-OVA (ATCC #CRL-2113) were purchased from the American Type Culture Collection. B16-OVA was kindly gifted from the laboratory of Dr. Paulo Rodriguez. Cells were subcultured when growth reached 80-90% confluency. For adherent cell lines, cells were detached using Trypsin-EDTA (0.25%) (Gibco #25200056) for three minutes at 37° C., 5% CO₂.

Complete RPMI-1640 (cRPMI-1640) was used as the base for all cell cultures. Media was prepared using RPMI-1640 (Lonza BioWhittaker #12167F) with 10% heat-inactivated fetal bovine serum (FBS) (Gemini #100106), 25 mM HEPES (Gibco #15630080), 1% penicillin-streptomycin (Gibco #15140122), 3 mM UltraGlutamine I (Lonza #BE17-605E/U1), and 0.05 mM 2-mercaptoethanol (MP Biomedicals, #194834). Culture media for T lymphoma EL-4, E.G7-OVA, and gp100 pulsed EL-4 consisted of cRPMI-1640 supplemented with 1 mM sodium pyruvate (Gibco #11360070). Culture media for B16-OVA consisted of cRPMI-1640 supplemented with 0.8% G418 (Gibco #10131035)

### In Vitro Primary T Cell Activation and Pharmacological Treatment

Lymph nodes and spleen were isolated from wild-type C57BL/6N mice. Tissue was mechanically dissociated with the back of a syringe plunger and passed through a 70 M filter. CD3 T cells (Invitrogen #8804-6820-74) or CD8 T cells (Invitrogen #8804-6822-74) were enriched using a negative magnetic bead selection. 24-well plates (Corning #3524) were coated with either 0.1, 0.5, or 1 g/ml anti-mouse CD3e (BD Biosciences #553058, clone 145-2C11) and anti-mouse CD28 (BD Biosciences #553294, clone 37.51) suspended in PBS for two hours at 37° C., 5% C02. PBS containing coating antibodies were then removed and 1×10⁶ T cells in 2 ml cRPMI-1640 was added to each well. GPR84 antagonist GLPG1205 was purchased (MedChemExpress #HY-135303) or provided by Galapagos NV. GPR84 agonist DL175 was purchased from Tocris Bioscience (#7082). For treatment, drugs were dissolved in DMSO (Fisher #BP231-100), and then diluted with cRPMI-1640 to an in vitro working concentration of 3 μM or 10 μM. Treated cells were incubated at 37° C., 5% CO₂ for the designed study duration.

### In Vitro Primary BM-MDSC Differentiation and Pharmacological Treatment

Bone marrow cells were isolated by flushing the tibia and femurs of wild-type C57BL/6N mice. Bone marrow was pelleted and resuspended in ACK lysis buffer (Quality Biological #118156101) for three minutes to lyse red blood cells. Purified leukocytes were then pelleted and resuspended at a concentration of 1×10⁶ cells/ml in cRPMI-1640 containing 40 ng/ml G-CSF (Gemini #300-307P), GM-CSF (Gemini #300-308P), and IL-6 (Gemini #300-327P) to stimulate bone marrow myeloid derived suppressor cell (BM-MDSC) differentiation. To generate BM-MDSCs, 3×10⁶ cells were plated in a 6-well dish and incubated at 37° C., 5% CO₂ for 96 hours. For treatment, drugs were administered at either 3 μM or 10 μM concentrations and added to the BM-MDSC cell culture well 24 hours after plating.

### Immunoblotting

Harvested cells were washed 1× with cold PBS and lysed in egg lysis buffer with 1× Halt protease and phosphatase inhibitor cocktail (Thermo #1861280) for 15 minutes on ice. Lysates were clarified by centrifugation at 12,500 RPM for 10 minutes, transferred to a clean 1.5 ml Eppendorf tube, and stored at −80° C. until analysis. Protein concentration was quantified using a BCA protein assay kit (Thermo #A53225). 20 g of protein diluted with 1×LDS sample buffer (Life Technologies #B0008) and 1×sample reducing agent (Life Technologies #B0009), were denatured at 95° C. for 5 minutes. Denatured T cell protein was resolved using a 10% Bolt Bis-Tris plus protein gel (Thermo #NW00102BOX) and denatured BM-MDSC protein was resolved using an 8% Bolt Bis-Tris plus protein gel (Thermo #NW00082BOX). Gels were transferred to a PVDF membrane (Invitrogen #IB401002) using a seven-minute P3 program on an Invitrogen iBlot dry transfer system. Membranes were then blocked for one hour using 5% nonfat dry milk in TBS-T, washed three times with TBS-T and probed with primary antibody diluted with 5% BSA in TBS-T overnight at 4° C. with gentle rocking. Primary antibody was removed, and membranes were washed three times with TBS-T followed by secondary antibody diluted in TBS-T at room temperature for one hour with gentle rocking. Membranes were washed three times with TBS-T and film developed with ECL western blotting substrate (Thermo #32106). Membranes were reprobed after stripping for seven minutes at room temperature (Thermo #21059) or after inactivation of horseradish peroxidase with 30% hydrogen peroxide for 15 minutes at room temperature (Fisher #H323-500). Primary antibodies used were rabbit anti-GPR84 (1:300) (Thermo #BS-15353R), rabbit anti-granzyme B (1:10,000) (CST #4275S), rabbit anti-perforin (1:1000) (CST #62550S), mouse anti-arginase-I (BD Biosciences #610708), mouse anti-iNOS (BD Biosciences #610432), and mouse anti-R-actin (1:500,000) (Sigma #A228). Secondary antibodies used were anti-mouse-HRP (Kindle Biosciences #R1005) and anti-rabbit-HRP (Kindle Biosciences #R1006). Analysis was conducted using ImageJ (NIH Bethesda, MD) and normalized to the housekeeping protein β-actin.

### Immune Profiling by Flow Cytometry

Single cell suspensions were either stained with Fc block (BD Biosciences #553142) for in vitro assays or Fc block in combination with Live/Dead Near-IR (Invitrogen #L10119) for in vivo assays for 15 minutes at room temperature. Cells were pelleted and subsequently stained with a selected panel from the antibodies anti-CD69 (BD Biosciences #746813, clone H1.2F3), anti-CD25 (BD Biosciences #553072, clone 7D4), anti-CTLA-4 (BD Biosciences #564332, clone UC10-4F10-11), anti-LAG-3 (BD Biosciences #745214, clone C9B7W), anti-PD-1 (BD Biosciences #566514, clone J43), anti-CD62L (BD Biosciences #612833, clone MEL-14), anti-CD44 (BD Biosciences #741057, clone IM7), anti-CD45 (BD Biosciences #612924, clone 30-F11), anti-CD3 (BD Biosciences #557984, clone 500A2), anti-CD4 (BD Biosciences #612900, clone GK1.5), anti-CD8 (BD Biosciences #563786, clone 53-6.7), SIINFEKL Tetramer (SEQ ID NO: 1) (NIH Tetramer Facility Atlanta, GA), anti-Gr-1 (BD Biosciences #562709, clone RB6-8C5), anti-Ly-6G (BD Biosciences #560599, clone 1A8), anti-Ly-6C (BD Biosciences #560592, clone AL-21), anti-CD11b (BD Biosciences #552850, clone M1/70), or anti-F4/80 (BD Biosciences #565612, clone T45-2342) for 30 minutes at 4° C. protected from light. Cells were washed pelleted and fixed with 4.2% formaldehyde (BD Biosciences #554722) for 10 minutes at 4° C. After fixation, cells were pelleted and permeabilized with perm/wash buffer (BD Biosciences #554723) for 20 minutes at 4° C. Cells were pelleted and intracellularly stained with anti-IFNγ (BD Biosciences #563773, clone XMG1.2) for 30 minutes at 4° C. protected from light. After staining, cells were pelleted, resuspended and run by a BD FACSymphony A3 flow cytometer.

### CFSE Labeling and T Cell Proliferation

Isolated CD3 T cells were labeled with 1 μM CFDA SE (Thermo #V12883) for 15 minutes at 37° C. protected from light. After incubation, equal parts PBS was added, cells were pelleted, resuspended in cRPMI-1640, and further incubated for 20 minutes on ice to quench remaining CFDA. Cells were pelleted, resuspended and plated in an antibody coated well with the indicated drug concentrations at time zero. Activated T cells were incubated at 37° C., 5% CO₂ for 72 hours. After study duration, T cells were stained with anti-CD4, anti-CD8, anti-CD3 following the immune profiling by flow cytometry method and proliferation was measured by CFSE dilution.

### In Vitro Cytotoxicity

Treated OT-I CD8 T cells were prepared following the in vitro T cell activation method. Non-target EL4 cells (CFSE low—0.2 μM) and target E.G7-OVA cells (CFSE high—1 M) were prepared following the CFSE labeling protocol. Cells were subsequently washed, pelleted, resuspended at 1×10⁶ cells/ml, and mixed at 1:1 ratio. 100 μl of the mixed cells were plated into a 96-well flat bottom plate. In technical duplicate or triplicate OT-I T cells were added to wells to create an effector to target ratio of 3:1, 1:1, and 0.3:1 and incubated for 24 hours at 37° C., 5% C02. Cells were then harvested, CFSE positive cells were identified, and the percent specific lysis captured by flow cytometry and determined using the formula [1-(no effector control ratio/experimental ratio)]×100.

CRISPR-Cas9 GPR84 pmel-I T cells were prepared following the CRISPR-Cas9 method. Non-target EL4 cells (CFSE low—0.1 μM) and target EL4_{gp100} cells (CFSE high—1 M) were prepared. EL4_{gp100} CFSE high cells were prepared by pulsing EL4 with 1 g/ml gp100 (Anaspec #AS62589) and incubating for 30 minutes at 37° C., 5% C02. Cells were subsequently washed, pelleted, resuspended at 1×10⁶ cells/ml, and mixed at 1:1 ratio. 1×10⁵ of the mixed cells were plated into a 96-well round-bottom plate. In technical duplicate or triplicate GPR84 knockout or no target control CD8 μmel-I T cells were added to wells to create an effector to target ratio of 0.5:1, 0.25:1, and 0.12:1 and incubated for 24 hours at 37° C., 5% C02. Cells were then harvested, stained with anti-CD8 to remove the T cell population and the percent specific lysis was captured by flow cytometry.

### MDSC Suppression Assay

24-well plates were coated with 1 g/ml anti-mouse CD3e and anti-mouse CD28 resuspended in PBS for two hours at 37° C., 5% C02. PBS containing coating antibodies were then removed and 1×10⁶ naïve CFSE CD3 T cells in 1 ml was added to each well. Treated BM-MDSCs were removed using a rubber policeman. Cells were pelleted, resuspended in cRPMI-1640 and added to each T cell containing well at 1.25×10⁵ cells in 1 ml, generating an 8:1 CD3 to MDSC ratio. Co-cultures were incubated for 72 hours at 37° C., 5% C02. Cells were harvested and stained following the immune profiling by flow cytometry method. MDSC suppressive capacity was measured by their ability to inhibit CD3 T cell proliferation measured by CFSE dilution.

### CRISPR-Cas9 Knockout of GPR84 Receptor in Primary T Cells

Isolated pmel-I splenocytes were plated at a density of 2.5×10⁶ in 1 ml of cRPMI-1640 containing 300 ng/ml anti-mouse CD3 (Biolegend #100340, clone 145-2C11), 500 IU/ml IL-2, 5 ng/ml IL-7 (PreproTech #217-17), and 100 ng/ml IL-15 (PreproTech #210-15) into a 24-well plate. Cells were incubated at 37° C., 5% CO₂ for 48 hours. Cells were then pelleted and suspended to a concentration of 4×10⁵ cells/ml in fresh cRPMI-1640 with 500 IU/ml IL-2 and cultured for an additional 24 hours at 37° C., 5% CO₂. Before electroporation, T media was prepared consisting of modified cRPMI-1640 (20% FBS, 500 IU/ml IL-2, and no antibiotics). 615 μl of fresh T media was pipetted into 24-well plates. Preparation of CRISPR-Cas9 complexes and electroporation of primary T cells were conducted following the Alt-R CRISPR-Cas9 system using the Neon Transfection System instructions provided by Integrated DNA Technologies (IDT Coralville, IO). The program used for electroporation consisted of 1600V, 10 ms, and three pulses. Electroporated cells were transferred to 24 well plates containing fresh T media and an additional 525 μl of T media was added after 24 incubation at 37° C., 5% CO₂. Cells were counted 48 hours after electroporation and cRPMI-1640 supplemented with 500 IU/ml IL-2 was added to adjust the cell density to 4×10⁵ cells/ml. CRISPR-Cas9 knockout cells were expanded every 48 hours by adjusting cell density to 4×10⁵ cells/ml in 24-well plates. Alt-R CRISPR-Cas9 no target control crRNA and GPR84 crRNA were purchased from IDT. GPR84 crRNA sequence is listed in Table 1.

TABLE 1
CRISPR-Cas9 guide RNA sequence (crGPR84)

| Name | Sequence |
|---|---|
| Mm.Cas9.GPR84.1.AA | 5′-/AltR1/rCrUrC rGrArG rCrArC |
| | rUrGrG rArCrC rArArU rArCrG |
| | rUrUrU rUrArG rArGrC rUrArU |
| | rGrCrU/AltR2/-3′ (SEQ ID NO: 2) |

### Cytokine/Chemokine Quantification

Treated CD3 T cells were prepared following the in vitro T cell activation method and incubated for 72 hours at 37° C., 5% CO₂. Culture supernatant was harvested and clarified by centrifugation at 2000 RPMI for five minutes. Supernatant was then transferred to a clean 1.5 ml Eppendorf tube and stored at −80° C. until analysis. Cytokines and chemokines were quantified using a Milliplex mouse cytokine/chemokine magnetic bead panel (Millipore MCYTMAG-70K-PX32) on a Luminex MAGPIX system with undiluted samples following the manufacturer's instructions.

### ATP Rate Assay

Treated CD8 T cell were prepared following the in vitro T cell activation method and incubated for 72 hours at 37° C., 5% CO₂. Treated BM-MDSCs were prepared followed the in vitro BM-MDSCs differentiation method. The XFe ATP rate assay preparation and analysis was conducted following the manufacturer's user guide and instructions. (Agilent #103592-100) On day of analysis, XFe cell culture microplate was coated with 22.4 g/ml CellTak (Corning #354240) for 20 minutes at room temperature. Treated cells were removed from culture, pelleted and resuspended in warm assay medium at a concentration of 3×10⁶ cells/ml (1.5×10⁵ cells/well) for CD8 T cells and 2.6×10⁶ cells/ml (1.3×10⁵ cells/well) for BM-MDSCs. Cells were adhered to the culture plate by centrifugation 200×G, for one minute with zero brake per the manufacturer's instructions for immobilization of non-adherent cells (Agilent, Santa Clara, CA). After XFe ATP rate assay was complete, wells that did not receive injections of oligomycin and/or rotenone/antimycin A were removed before analysis. Results were procured using the Seahorse XF real-time ATP rate assay report generator.

### 2-NBDG Glucose Analog Uptake Assay

Treated CD3 T cells were prepared following the in vitro T cell activation method and incubated for 72 hours at 37° C., 5% CO₂. Cells were washed, pelleted and resuspended in cRPMI-1640 with 30 M 2-NBDG. (Thermo #N13195) Cells were then incubated for one hour at 37° C., 5% CO₂. After incubation, T cells were washed, pelleted and stained with anti-CD4, anti-CD8, anti-CD3 following the immune profiling by flow cytometry method. Percent uptake was calculated using mean fluorescent intensity (MFI).

### RNA Extraction and Quantitative PCR

Treated CD8 T cells were prepared following the in vitro T cell activation method and incubated for 48 hours at 37° C., 5% CO₂. Cells were washed, pelleted, and lysed with buffer RLT containing 2-mercaptoethanol (Sigma #M7154-24ML). Lysed cells were then added to a QIAshredder (Qiagen #79656) column and tissue was homogenized by centrifugation at 12,000 RPM for two minutes at room temperature. RNA was extracted using a RNeasy mini kit (Qiagen #74106) following the manufacturer's instructions with an on-column DNase digestion step (Qiagen #79254). 1 g RNA was converted to cDNA using a Verso cDNA synthesis kit (Thermo #AB1453/B) with a MJ Research peltier thermocycler following the manufacturer's instructions. Quantitative PCR was conducted using a 5 μl reaction volume in a 384-well plate (Applied Biosystems #4309849) containing 10 ng of template cDNA, lx SYBR green PCR master mix (Applied Biosystems #A25742), 0.1 μM forward primer (IDT Coralville, IO), 0.1 M reverse primer (IDT Coralville, IO), and nuclease free water. (Invitrogen #AM9938) PCR amplification was performed in technical triplicate using an Applied Biosystems QuantStudio 12K flex system. Assay parameters used a hold stage followed by a PCR stage. The hold stage parameters were 50° C. for two minutes followed by 95° C. for 10 minutes. The three-step PCR stage consisted of 95° C. for 15 seconds, 58° C. for 15 seconds, and 72° C. for one minute for 40 cycles. Relative fold changes were calculated using ΔΔCt normalized to the housekeeping gene P3-actin. List of mouse PCR primers for enzymes in glycolysis are listed in Table 2.

TABLE 2
enzyme primers sequences

| | | SEQ |
|---|---|---|
| | | ID |
| | | NO: |
| Name | Sequence | [ ] |
| HK1 forward | 5′-AGGATGACCAAGTCAAAAAGATTG | 3 |
| HK1 reverse | 5′-GGAATGGACCGGACAAAGGT | 4 |
| HK2 forward | 5′-GTTTCTCTATTTGGCCCCGAC | 5 |
| HK2 reverse | 5′-AGAGATACTGGTCAACCTTCTGC | 6 |
| GPI1 forward | 5′-AACTCTTGCCACACAAGGTCT | 7 |
| GPI1 reverse | 5′-TCCCACATGATGCCCTGAAC | 8 |
| PFKP forward | 5′-ATGGTTCCTGCTACTGTCTCCA | 9 |
| PFKP reverse | 5′-TTAATGCGGTCGCACGTGTCT | 10 |
| ALDOA forward | 5′-CTTAGTCCTTTCGCCTACCCA | 11 |
| ALDOA reverse | 5′-GCGGGTCATGTTGAAGCTG | 12 |
| TPI1 forward | 5′-ACCCGGATCATTTATGGAGGTTC | 13 |
| TPI1 reverse | 5′-GGCAGTGCTCATTGTTTGGC | 14 |
| GAPDH forward | 5′-ACCACAGTCCATGCCATCAC | 15 |
| GAPDH reverse | 5′-TCCACCACCCTGTTGCTGTA | 16 |
| PGAM1 forward | 5′-GCACTGCCCTTCTGGAATGA | 17 |
| PGAM1 reverse | 5′-CCTCTTCTGAGAGACCCTCCA | 18 |
| PKM2 forward | 5′-GCAGCGACTCGTCTTCACT | 19 |
| PKM2 reverse | 5′-GCATGGTTCCTGAAGTCCTCG | 20 |
| LDHA forward | 5′-GGAGTGGTGTGAATGTTGCC | 21 |
| LDHA reverse | 5′-GATCACCTCGTAGGCACTGTC | 22 |
| ß-actin | 5′-GATCAAGATCATTGCTCCTGA | 23 |
| forward | | |
| ß-actin | 5′-CAGCTCAGTAACAGTCCGCC | 24 |
| reverse | | |

### Single-Cell RNA Sequencing and Analysis

Treated CD8 T cells were prepared following the in vitro T cell activation method and incubated for 16 hours at 37° C., 500 CO₂. Treated T cells were mixed with treated BM-MDSCs at a 70% o (T cell): 30% (BM-MDSC) ratio moments before analysis. 10,000 cells with greater than 90% viability were used in the analysis at a concentration of 1,000 cells/μl. Barcoded cDNA libraries were prepared using a chromium next GEM single-cell 3′ reagent kit v3.1 (10× Genomics #1000127) following the manufacturer's instructions. Libraries were validated using a high sensitivity DNA kit (Agilent #50674626) using a 2100 bioanalyzer. (Agilent, Santa Clara, CA) DNA was subsequently quantified by fluorometry using a Qubit dsDNA high sensitivity assay kit (Invitrogen #Q32854). DNA libraries were pooled in equimolar ratios and sequenced using an Illumina NextSeq 500 (Illumina, San Diego, CA) following 10× Genomics' instructions. For analysis, dying cells (>12% o mitochondrial gene expression) and cells with >6000 or <200 unique genes expressed were removed. Doublets were subsequently removed using DoubletFinder (https://github.com/chris-mcginnis-ucsf/DoubletFinder). An unsupervised clustering and plotting using UMAPs was completed on the remaining cells. CD8 T cells were identified by first conducting CD3 followed by CD8 refinement and reclustering. To characterize the CD8 subsets, the top 50 differentially expressed genes between each subcluster were compared against publicly available datasets, and known genes associated with activation, exhaustion, and memory T cell states. To determine changes in metabolism and cytotoxicity, a curated list of genes associated with glucose, fatty acid, lipid metabolism, and cytotoxicity were generated. Average gene expression by treatment group was determined for each gene and visualized by heatmap.

### Animal Models

C57BL/6N mice were bred in-house or purchased from Envigo (Indianapolis, IN). OT-I mice were bred in-house, and pmel-I mice were purchase from Jackson Laboratory (Bar Harbor, ME). Purchased mice were held for one week prior to use.

### In Vivo Adoptive Cellular Transfer and Tumor Analysis

C57BL/6N mice were injected with 3×10⁵ B16-OVA or B16 subcutaneously in the right flank. Five days after injection, mice were administered 200 μg cyclophosphamide monohydrate (Sigma #C73971G) dissolved in PBS by intraperitoneal injection to induce minor lymphodepletion. 48 hours after lymphodepletion, mice were intravenously adoptively transferred 2.5×10⁶ CD8 OT-I or CD8 μmel-I pre-treated ex vivo following the in vitro T cell activation method. Tumor growth rates were determined by tumor volume measurement beginning on day four and continuing three times per week for the study duration. Tumor volumes were determined by the ellipsoid formula: [(Length×Width²)/2]. At the end of study, tumors were either fixed in 10% formalin or dissociated into a single-cell suspension for flow cytometry. Single-cell suspension was performed by chopping tumor tissue with scissors followed by a 30-minute incubation at 37° C. with 100 g/ml DNase I (Roche #10104159001) and 0.5 U/ml Liberase (Roche #05401127001) in PBS. Solution was then diluted with cRPMI-1640 and passed through a 70 μm cell strainer, pelleted and resuspended in ACK lysis buffer to remove erythrocytes. Cells were then counted, filtered through a 70 μm cell strainer, and 10×10⁶ cells were aliquoted and stained following the immune profiling by flow cytometry method.

### In Vivo Vaccination Antigen Titration

C57BL/6N mice were first administered 200 μg cyclophosphamide dissolved in PBS by intraperitoneal injection to induce minor lymphodepletion. 48 hours after lymphodepletion, mice were intravenously adoptively transferred 2×10⁶ CFSE labeled CD8 OT-I T cells. Five hours later, mice were vaccinated by 100 μl subcutaneous injection with reducing doses of SIINFEKL (SEQ ID NO: 1) (Bachem #4033142.0001) in incomplete Freund's adjuvant (IFA) (Thermo #77145). Study ended 96 hours post-vaccination and lymph node tissue was isolated for analysis. Single-cell suspensions of lymph node were stained following the immune profiling by flow cytometry method. Vaccine effectiveness was determined by the percent proliferation of adoptively transferred cells. The sub-optimal vaccine dose was then selected as the amount of antigen that elicited a 50% response.

### Immune Profiling and Proliferation of In Vivo Vaccinated CD8 T Cell

C57BL/6N mice were first administered 200 μg cyclophosphamide dissolved in PBS by intraperitoneal injection to induce minor lymphodepletion. 48 hours after lymphodepletion, mice were intravenously adoptively transferred 2×10⁶ CFSE labeled CD8 OT-I T cells. Five hours later, mice were vaccinated by subcutaneous injection with 0.07 g SIINFEKL (SEQ ID NO: 1) in IFA. 16 hours after vaccination, mice were administered 30 mg/kg or 90 mg/kg GLPG1205 suspended in 0.5% methylcellulose (Sigma #M0430100G) with 0.5% Tween-80 (Sigma #P4780100ML) (MC) orally once daily for three days. 96 hours post-vaccination, lymph node and spleen tissue were isolated for analysis. Single-cell suspensions were treated with ACK lysis to remove red blood cells, and then stained following the immune profiling by flow cytometry method. Treatment effectiveness was determined by the percent proliferation and PD-1 expression of adoptively transferred cells.

### In Vivo Therapeutic Vaccination and Tumor Analysis

C57BL/6N mice were injected with 3×10⁵ B16-OVA subcutaneously in the right flank. Five days after injection, mice were administered 200 μg cyclophosphamide dissolved in PBS by intraperitoneal injection to induce minor lymphodepletion. 48 hours after lymphodepletion, mice were intravenously adoptively transferred 2.5×10⁶ labeled CD8 OT-I T cells. Five hours later, mice were vaccinated by subcutaneous injection with 0.07 g SIINFEKL (SEQ ID NO: 1) in IFA. 16 hours after vaccination, mice were administered 30 mg/kg GLPG1205 suspended in MC orally once daily for three days. 96 hours post-vaccination, mice were subsequently administered two more series consisting of a 0.07 μg SIINFEKL (SEQ ID NO: 1) in IFA vaccination boost followed by three days of oral 30 mg/kg GLGP1205. Tumor growth rates were determined by tumor volume measurement beginning on day four and continuing three times per week for the study duration. Tumor volumes were determined by the ellipsoid formula: [(Length×Width²)/2].

### In Vitro Apoptosis Detection

Treated CD3 T cells were prepared following the in vitro T cell activation method and incubated for 72 hours at 37° C., 5% CO₂. Cells were washed, pelleted and stained with anti-CD4, anti-CD8, anti-CD3, annexin V (BD Biosciences #550474), and propidium iodide (BD Biosciences #556463) in Annexin V binding buffer following the manufacturer's instructions (BD Biosciences #556547)

### Statistical Analysis

Statistical significance was determined using either an unpaired two-tailed t-test or a two-way ANOVA with or without Dunnett's post-hoc test in Graphpad Prism 6 (Graphpad San Diego, CA). Results are displayed as the mean with standard deviation or standard error described in the figure legend. The degrees of significance are defined by: *p≤0.05, **p≤0.01, ***p≤0.001, ****p≤0.0001.

### Results

GPR84 can have the ability to both modulate myeloid activity and regulate metabolic homeostasis.^{3,14,15} These results indicate how GPR84 affects the differentiation, activation, and function of immune cells. We show that these changes are in part due to GPR84's ability to modulate the metabolic state. We show how GPR84 mediated metabolic reprogramming leads to multiple new immunotherapies for cancer treatment.

### The Effect of GPR84 Modulation on Immune Cell Activity

### GPR84 Receptor Modulation Alters Proliferation and Cytotoxic Activity of CD8 T Cells

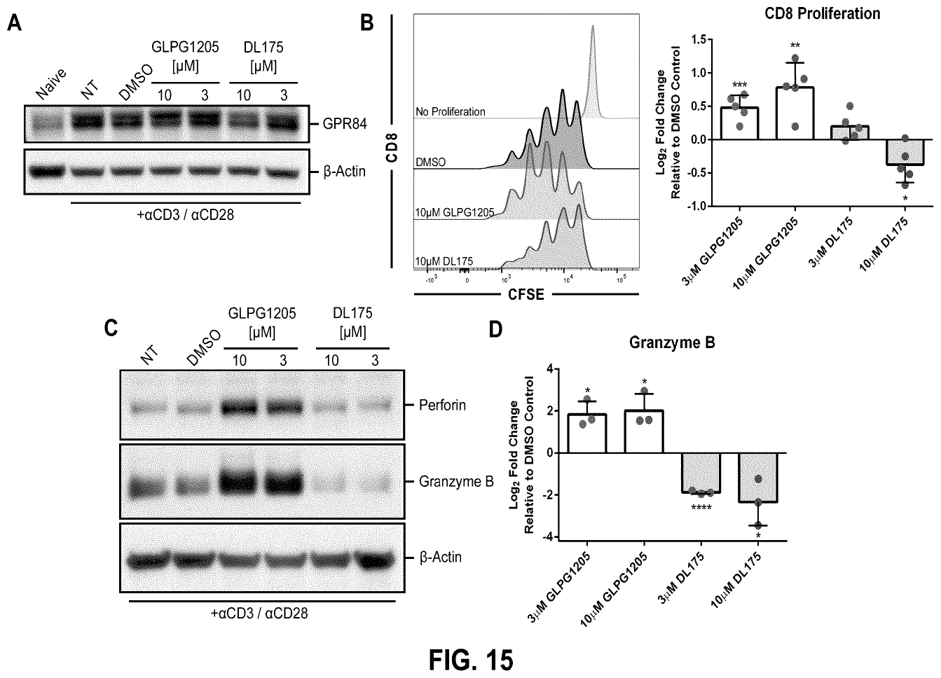

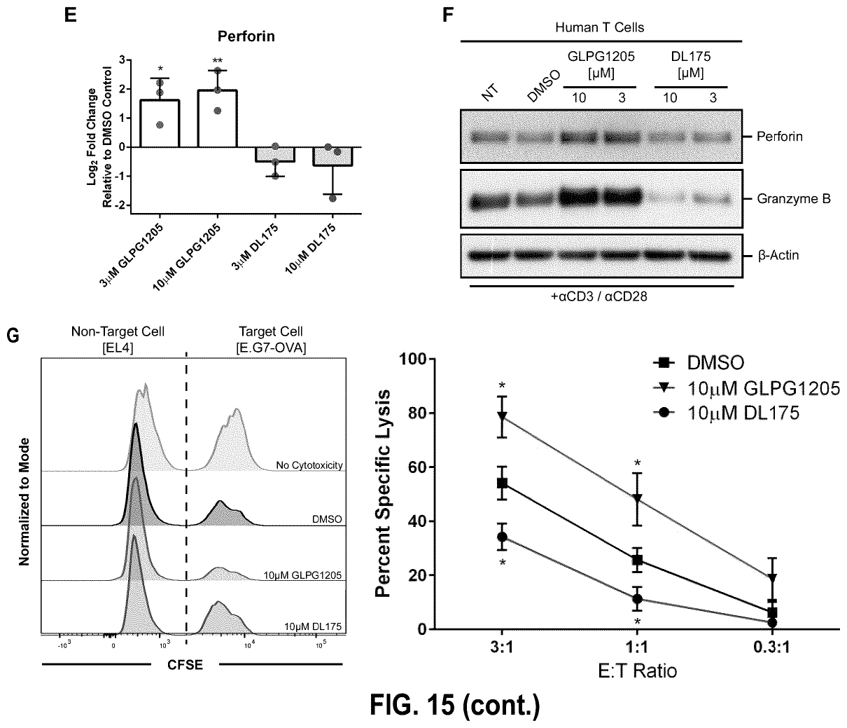

Without wishing to be bound by theory, GPR84 can metabolically affect the T cell compartment and regulate activity. We determined GPR84 expression on both naïve and activated CD3 T cells. We found that basal expression of GPR84 on naïve T cells dramatically upregulated after anti-CD3/anti-CD28 stimulation. Without wishing to be bound by theory, pharmacological modulation with GPR84 antagonist GLGP1205 or agonist DL175 can affect expression. To assess this, we treated activated CD3 T cells with 3 μM or 10 μM GPR84 antagonist GLPG1205 or 3 μM or 10 μM GPR84 agonist DL175. While 10 M of the agonist DL175 led to a reduced trend in GPR84 expression, repeated biological experiments found that modulation of the receptor with antagonist or agonist did not significantly affect GPR84 expression. (FIG. 15 Panel A). Knowing that GPR84 was present on CD3 T cells, we can determine how receptor modulation can affect the activity of CD8 T cells. In one approach we can examine if regulating the GPR84 receptor affected CD8 T cell proliferation. To test this, we activated naïve T cells with anti-CD3/anti-CD28 in the presence or absence of GPR84 antagonist GLGP1205 or agonist DL175. We found that treatment with 3 μM GPR84 antagonist GLPG1205 significantly increased proliferation (0.48 Log₂ fold change, or 39.3%), and this increase can be further amplified by 0.78 Log₂ fold change, or 71.7%, when T cells were exposed to 10 μM GLPG1205. In contrast, CD8 T cells exposed to 3 μM GPR84 agonist DL175 did not significantly affect proliferation, but when exposed to 10 μM DL175, proliferation significant reduced by −0.37 Log₂ fold change, or 29.4%. (FIG. 15 Panel B).

To determine how GPR84 affected CD8 cytotoxicity, we examined the effect that GPR84 modulation had on cytotoxic protein production. Once again, we activated T cells in the presence of low and high concentrations GPR84 antagonist GLPG1205 and agonist DL175. After treatment, we determined granzyme B and perforin by western blot. (FIG. 15 Panel C) We found that treatment with 3 μM GLPG1205 significantly increased granzyme B protein production by 3.57 times (1.84 Log₂ fold change) compared to DMSO treated baseline control. Additionally, granzyme b production further increased to 4.04 times (2.01 Log₂ fold change) compared to DMSO when exposed to the higher 10 μM concentration. In contrast, when we examined the effect GPR84 agonist DL175, granzyme B significantly reduced at the 3 μM (3.69 times or −1.88 Log₂ fold change) and further reduced by 5.11 times (−2.35 Log₂ fold change) at the 10 μM dose. (FIG. 15 Panel D) Similarly, when examining the pore-forming cytotoxic protein perform, we found 3 μM antagonist GLPG1205 significantly increased production 3.09 times (1.62 Log₂ fold change) and can be further improved at the 10 μM dose to 3.86 times (1.95 Log₂ fold change) compared to DMSO control. Unlike granzyme B, treatment with DL175 did not significantly reduce perform production. (FIG. 15 Panel E) We also observed these GPR84 mediated cytotoxic changes in human T cells. (FIG. 15 Panel F). After identifying the pro-inflammatory effects of GPR84 antagonist GLPG1205 and anti-inflammatory effects of GPR84 agonist DL175, we determined if these changes had cytotoxic functional effects. Using a cytotoxicity assay, we found that cells treated with 10 μM GLPG1205 had significantly improved killing capacity compared to DMSO control. In conjunction with previous evidence, CD8 T cells treated with DL175 had significantly reduced killing capacity, limiting their overall cytotoxic potential. (FIG. 15 Panel G)

### 4.1.2 GPR84 Receptor Modulation Reduces Apoptosis in CD8 T Cells

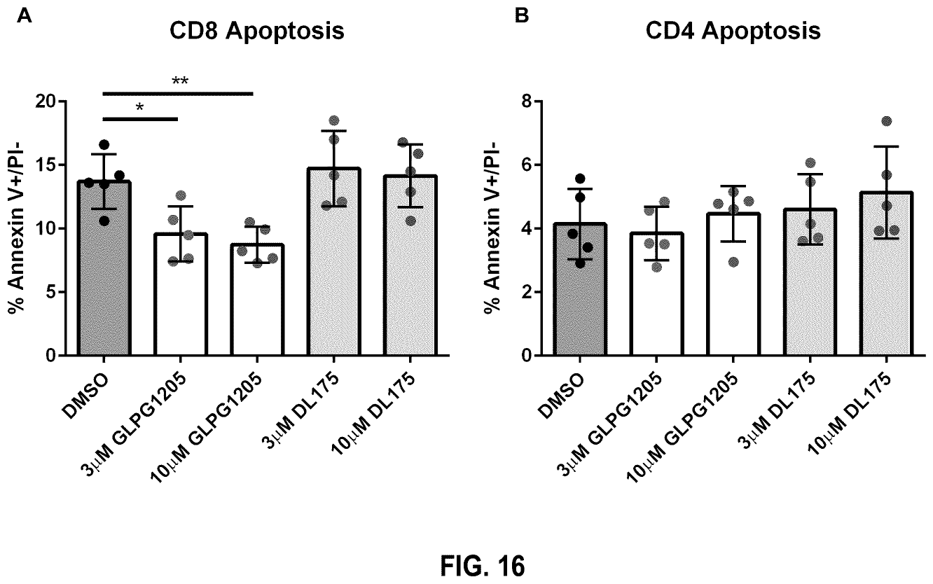

We conducted Annexin/PI apoptosis staining to ensure that the DL175 agonist induced reduction in proliferation was not due to T cell toxicity. Interestingly, using Annexin/PI staining we found that treatment with the antagonist GLPG1205 significantly reduced CD8 apoptosis, while treatment with the agonist DL175 displayed no measurable differences. (FIG. 16 Panel A) In addition, CD4 T cells treated with the antagonist GLPG1205 or agonist EL175 did not elicit any significant changes. (FIG. 16 Panel B)

### GPR84 Receptor Modulation Alters the Suppressive Capacity of BM-MDSCs

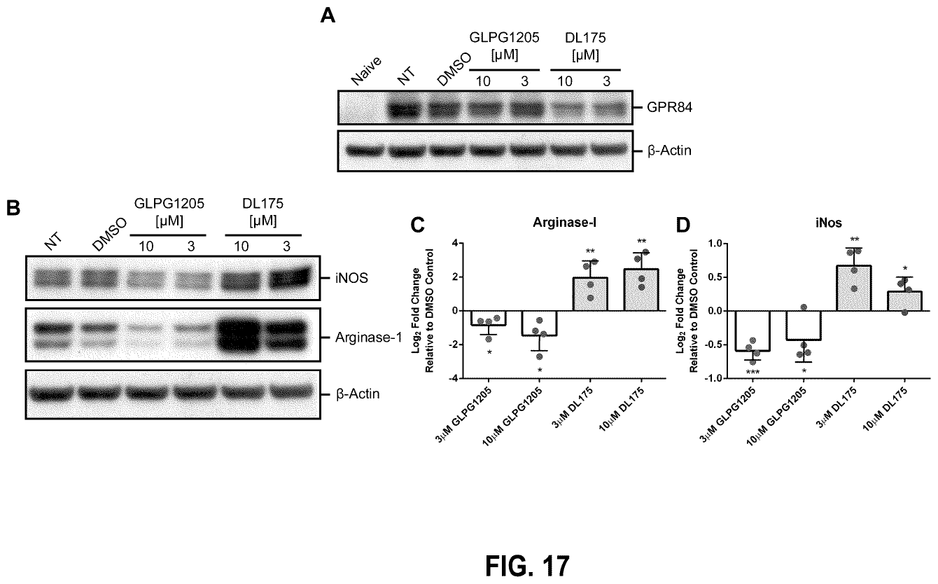

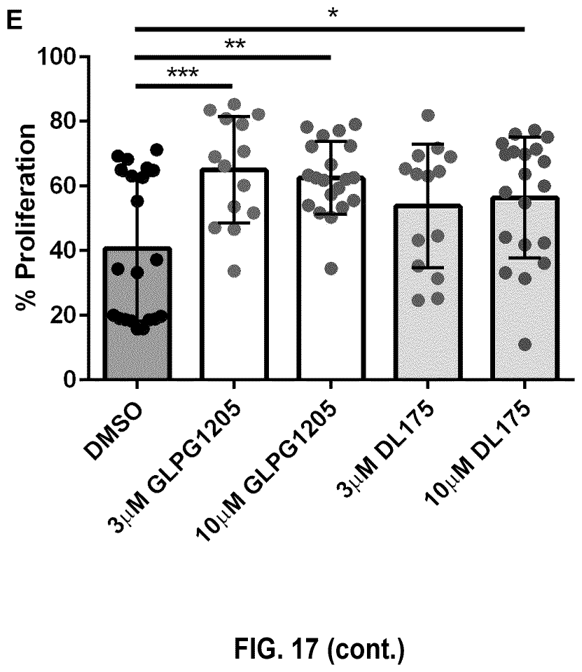

Since the tumor microenvironment does not only consist of T cells, we investigated the effect of GPR84 regulation on the important tumor infiltrating MDSC. MDSC's have an immunosuppressive characteristic, and so their presence is considered pro-tumorigenic. Since we observed the activating properties of GPR84 antagonism on CD8 T cells, we observed if the effect of GPR84 modulation can elicit a similar effect on a different immune cell type. Like T cells, we first had to determine GPR84 expression on MDSCs. To test this, we looked at GPR84 expression in fresh bone marrow and differentiated BM-MDSCs. We found that GPR84 expression was absent in fresh bone marrow, but expression levels significantly increased after differentiation into BM-MDSCs. Like T cells, we wanted to see if pharmacological modulation with GPR84 antagonist GLPG1205 or agonist DL175 can affect expression. We treated differentiating BM-MDSCs with 3 μM or 10 μM GPR84 antagonist GLPG1205 or 3 μM or 10 μM GPR84 agonist DL175. While treatment with both the antagonist GLPG1205 and agonist DL175 led to a reduced intensity in GPR84 expression, repeated biological experiments found that modulation only neared statistical significance. (FIG. 17 Panel A).

Knowing that GPR84 receptor was present on BM-MDSCs, we next wanted to examine the effect that GPR84 modulation had on immunosuppressive protein production. To test this, BM-MDSCs were subsequently treated with low and high dose antagonist GLPG1205 and agonist DL175. After treatment, we determined the amount of immunosuppressive arginase-I and inducible nitric oxide synthase (iNOS) by western blot. (FIG. 17 Panel B). Treatment with 3 μM GLPG1205 significantly reduced arginase-I levels by 1.79 times compared to DMSO control. Arginase-I production further decreased by 2.75 times when exposed to the 10 μM concentration. When we examined the effect of GPR84 agonist DL175 on arginase-I production, protein abundance significantly increased by 3.87 times at the 3 μM dose and further increased to 5.48 times at the higher 10 μM dose. (FIG. 17 Panel C). Similarly, when examining the other MDSC-associated immunosuppressive protein, iNOS, we found that GLPG1205 significantly reduced iNOS production by 1.5 times at 3 M and 1.35 times at the 10 μM dose. When we used the agonist DL175, iNOS significantly increased by 1.59 times at 3 μM dose and by 1.22 times at the higher 10 μM dose. (FIG. 17 Panel D) After identifying the ability to reduce immunosuppressive protein production with GLPG1205 and subsequently increase with DL175, we determined if these changes had functional effects on the immunosuppressive capacity. Using a suppression assay, we found that the suppressive capacity of BM-MDSC was reduced at 3 μM GLPG1205, allowing T cells to proliferate 64.99% compared to 40.73% in the DMSO control group. Likewise, 10 μM GLPG1205 had similar effects, with T cells able to proliferate 62.49%. Surprisingly, while immunosuppressive proteins increased with DL175 agonist treatment, the suppressive capacity reduced. BM-MDSC's treated with 3 μM DL175 allowed T cells to proliferate at an increased 53.78% compared to the 40.73% in DMSO, but this increase was not significant. However, BM-MDSCs treated with 10 μM DL175 did significantly increase T cell proliferation to 56.35%. (FIG. 17 Panel E)

GPR84 receptor modulation regulates CD8 T cell activation

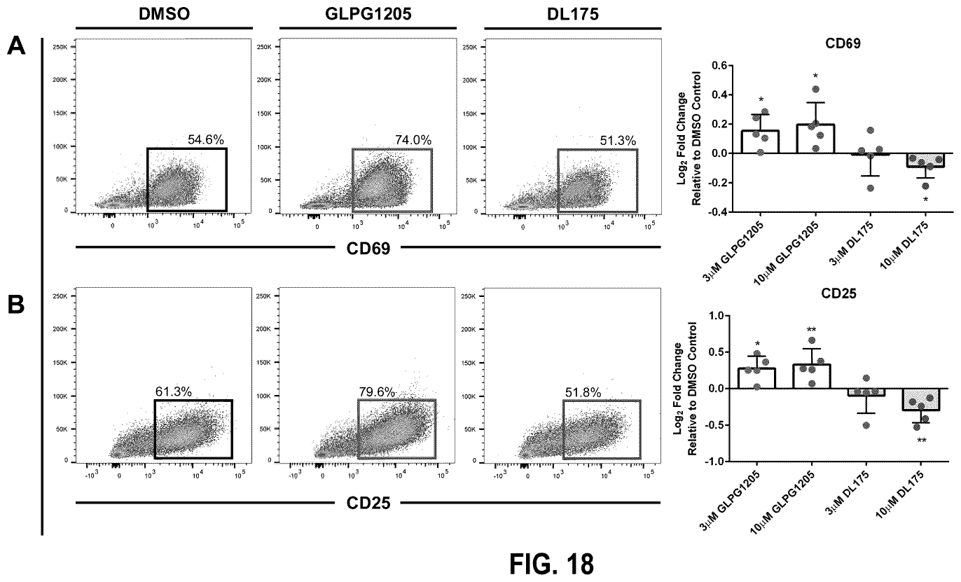

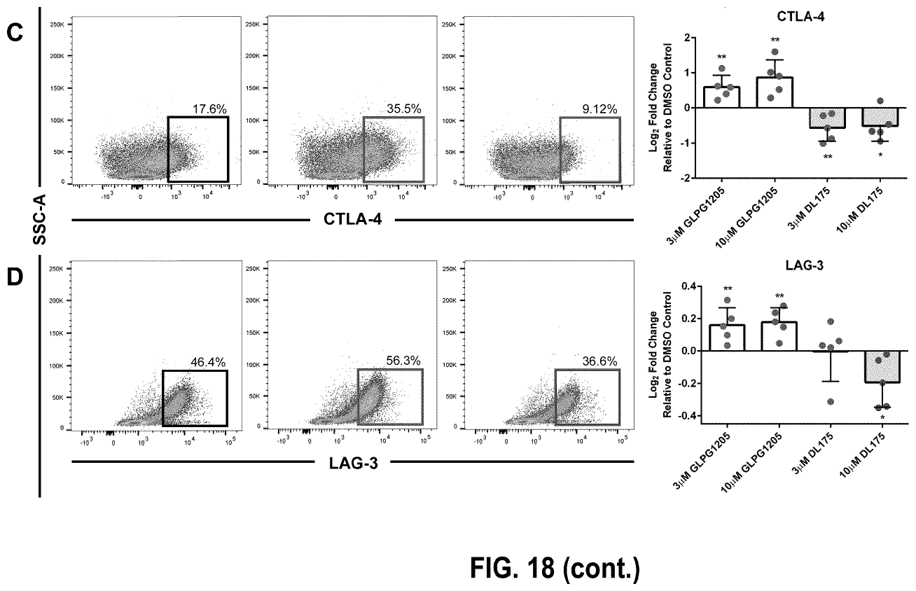

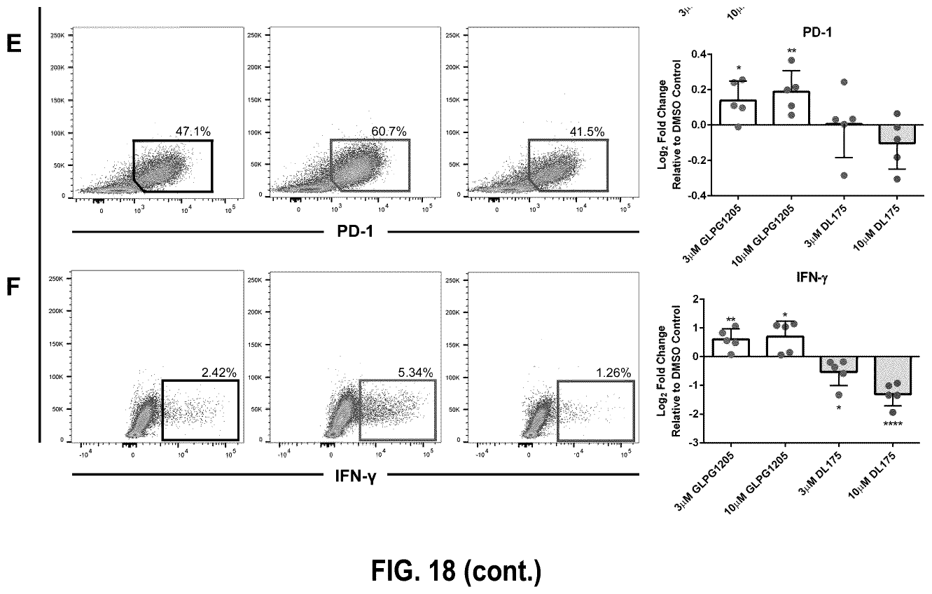

Since increased cytotoxic capacity can be associated with overall CD8 T cell activation, we explored the activity profile of these cytotoxic T lymphocytes. As T cells activate, they upregulate various intracellular and extracellular surface markers which aid in their expansion and overall activity. Using flow cytometry, we examined the various CD8 T cells markers of activation, and explored how GPR84 antagonist GLPG1205 and agonist DL175 affected overall expression. We examined the effect of GPR84 modulation on the early activation marker CD69. Both 3 μM and 10 μM GLPG1205 significantly increased CD69 expression by 11.24% and 14.59% respectively. Treatment with 3 μM DL175 did not lead to a significant reduction, however 10 μM was found to significantly reduce CD69 expression by 6.47%. (FIG. 18 Panel A) The next marker examined was the interleukin-2 receptor subunit a, CD25. Again, treatment with 3 μM and 10 μM GLPG1205 significantly increased CD25 expression by 21% and 25.58%. Like CD69, DL175 did not have a significant effect on CD25 expression at 3 μM, but significantly reduced expression levels by 22.95% when treated at the 10 μM concentration. (FIG. 18 Panel B) Next, we investigated the checkpoint regulators, CTLA-4, LAG-3, and PD-1. We found that 3 M GLPG1205 significantly increased CTLA-4 by 50.31%, LAG-3 by 11.72%, and PD-1 by 10.02%. At the higher 10 μM concentration, GLPG1205 increased CTLA-4 by 81.92%, LAG-3 by 13.15%, and PD-1 by 13.91%. When CD8 T cells were treated with the agonist DL175 at the 3 μM concentration, CTLA-4 was significantly downregulated by 48.31%, but elicited no effect on LAG-3 or PD-1. As seen before, when DL175 was used at the higher 10 μM concentration, CTLA-4 expression significantly reduced by 42.99% and LAG-3 by 14.37%. DL175 was not found to have a significant downregulation in PD-1 at the higher 10 μM concentration. (FIG. 18 Panels C-E) We also screened for the inflammatory type II interferon cytokine, interferon gamma (IFNγ). In concordance with the activation marker trends, treatment with GLPG1205 at both concentrations significantly increased intracellular IFNγ (3 M, 51.23%; 10 μM, 61.55%). In contrast, treatment with DL175 at both concentrations significantly reduced IFNγ levels (3 M, 45.04%; 10 μM, 148.72%). (FIG. 18 Panel F)

### GPR84 Receptor Modulation Regulates CD4 T Cell Activation

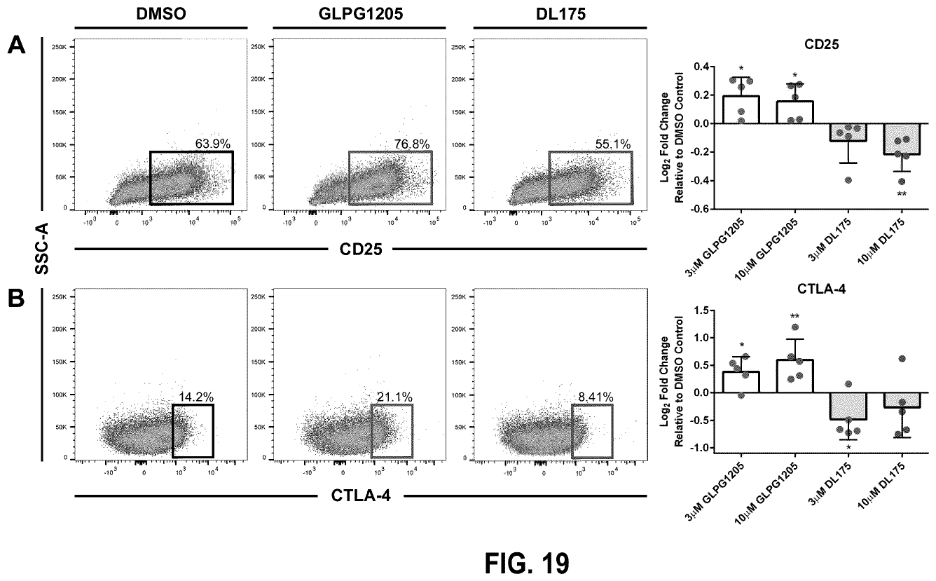

Since a robust anti-tumor effect also relies on an effective CD4 response, we investigated how GPR84 regulation also affected this T cell subset. Using flow cytometry, we examined various markers which aid in helper CD4 expansion and overall activity. We found that only two markers in CD4 T cells were significantly changed after GPR84 modulation. Compared to DMSO control, both 3 μM and 10 μM GLPG1205 significantly increased CD25 expression by 14.28% and 11.37% respectively. Using the agonist DL175 did not provide a significant difference at 3 μM but did significantly reduce CD25 expression by 16.15% at the higher 10 μM dose. (FIG. 19 Panel A) The other marker found to have significant changes was CTLA4. We found that treatment with 3 μM GLPG1205 significant increased expression by 30.31% and this was further increased to 51.16% in the 10 μM treatment group. In contrast, CTLA4 was significantly reduced by 39.78% with 3 μM DL175 agonist treatment, and while we observed an average reduction of 20.08% at the higher 10 μM dose, this change was not found to be significant. (FIG. 19 Panel B)

### GPR84 Receptor Modulation Regulates T Cell Cytokine Secretion

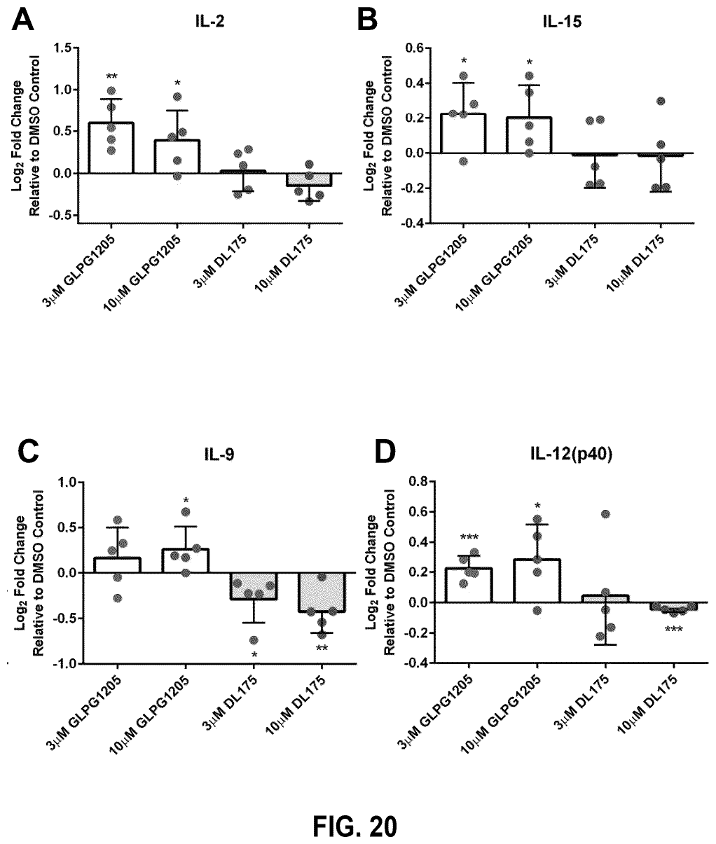

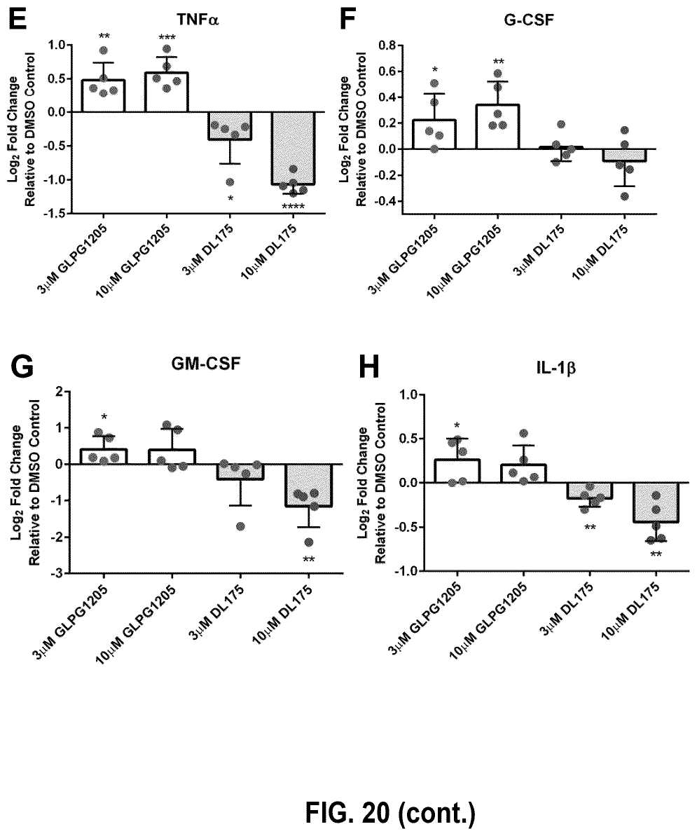

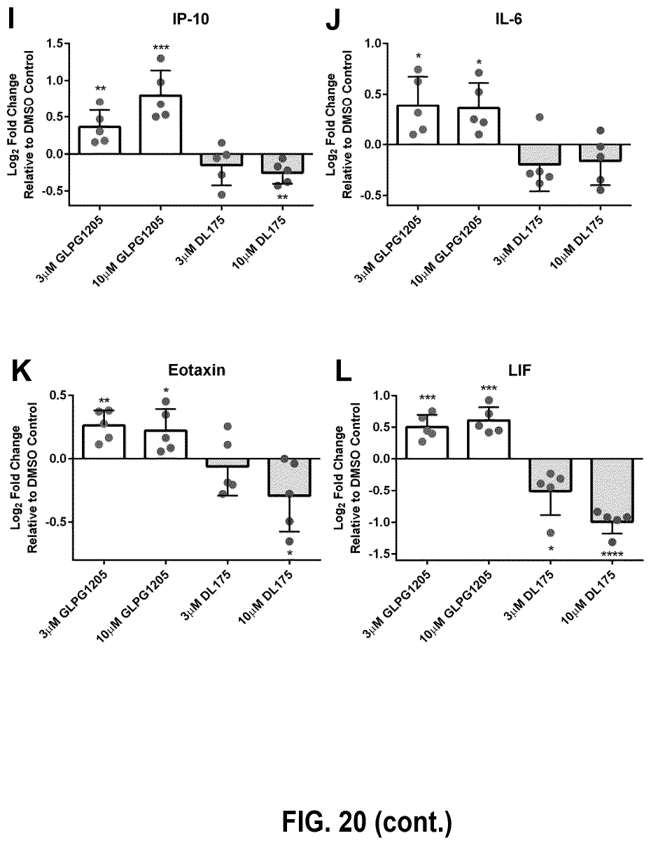

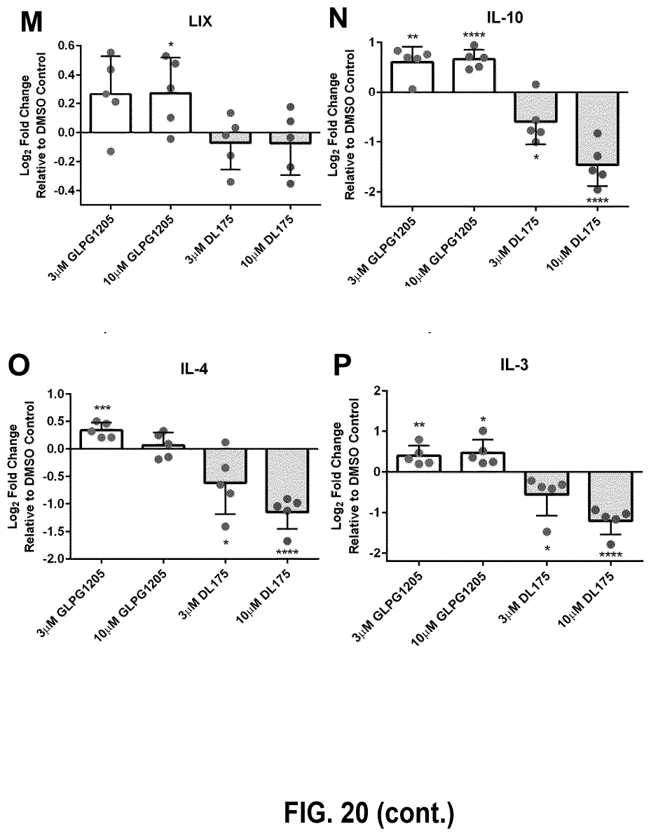

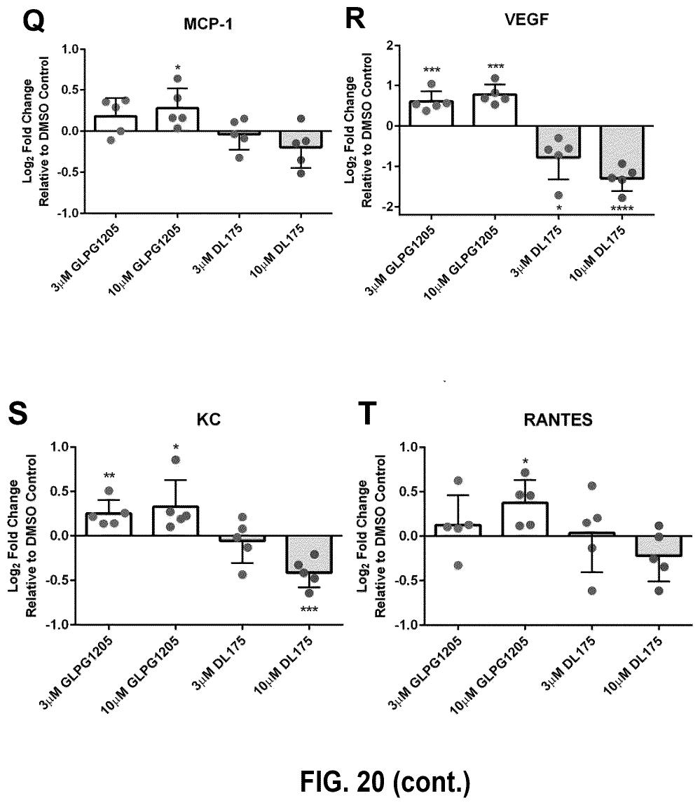

With evidence indicating that GPR84 can regulate T cell activation, we determined if this activation or suppression associated with changes in inflammatory, mitogenic, and T cell differentiating cytokine production. To do this, we looked at the mitogenic and T cells differentiating cytokines interleukin-2 (IL-2), interleukin-15 (IL-15), interleukin-9 (IL-9), interleukin-12 subunit p40 (IL-12(p40)) and tumor necrosis factor alpha (TNFα). IL-2, a cytokine which promotes the proliferation of T and B cells, GLPG1205 treatment significantly increased production by 51.36% at 3 M and 31.1% at 10 μM compared to DMSO control. In contrast, treatment with DL175 did not significantly reduce IL-2 at either dose. (FIG. 20 Panel A) Like IL-2, we saw similar changes in the cytokine IL-15. IL-15, a cytokine which promotes the proliferation of T and NK cells, significantly increased secretion after GLPG1205 antagonist treatment (3 M, 16.91%; 10 μM, 15.05%). Again, we did not see a significant change with DL175 agonist treatment compared to DMSO control. (FIG. 20 Panel B) We looked at IL-9, a cytokine which promotes proliferation of immune cells. We found that 3 M GLPG1205 antagonist treatment did not elicit any significant changes, but at 10 μM, IL-9 significantly increased by 19.94%. When examining the effect of GPR84 agonist DL175, 3 μM significantly reduced secretion by 22.34% and this was further increased to 33.98% at the higher 10 μM dose. (FIG. 20 Panel C) The next cytokine examined was IL-12(p40), a cytokine which promotes Th1 differentiation and survival. We found that GPR84 antagonist GLPG1205 significantly increased IL-12(p40) levels by 16.99% at 3 μM and this was further improved to 21.77% at the 10 μM dose. When the agonist DL175 was used, like previous cytokines, we did not observe a significant decrease at 3 μM but did observe a significantly minor decrease of 3.14% at 10 μM. (FIG. 20 Panel D) Subsequently, we observed treatment effects on TNFα, a pan-inflammatory cytokine which induces multiple functions including immune cell proliferation, differentiation, and activity. We found TNFα to significantly increase by 39.64% with 3 μM and by 50.91% with 10 μM GLPG1205 antagonist treatment. In opposite, we observed DL175 agonist to significantly reduce levels by 32.44% with 3 M and 109.39% with 10 μM. (FIG. 20 Panel E)

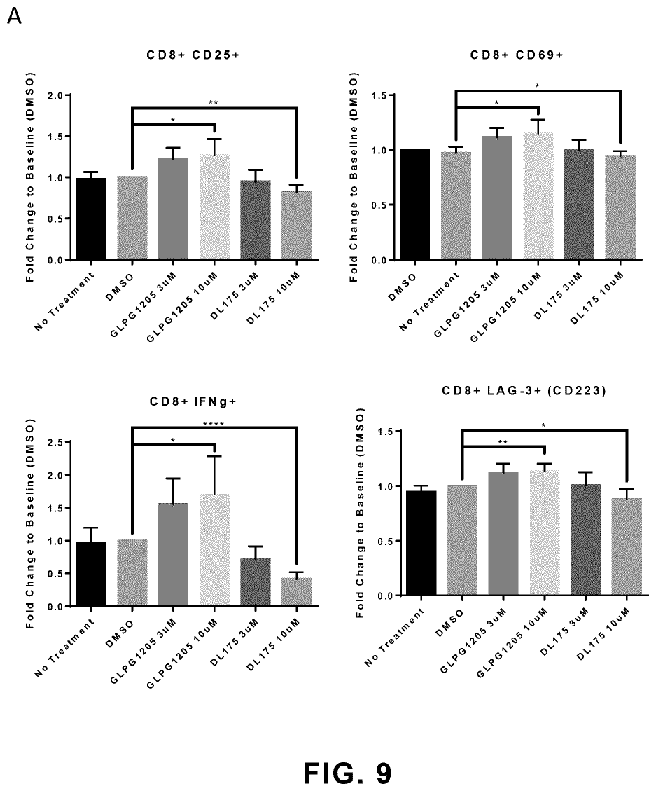

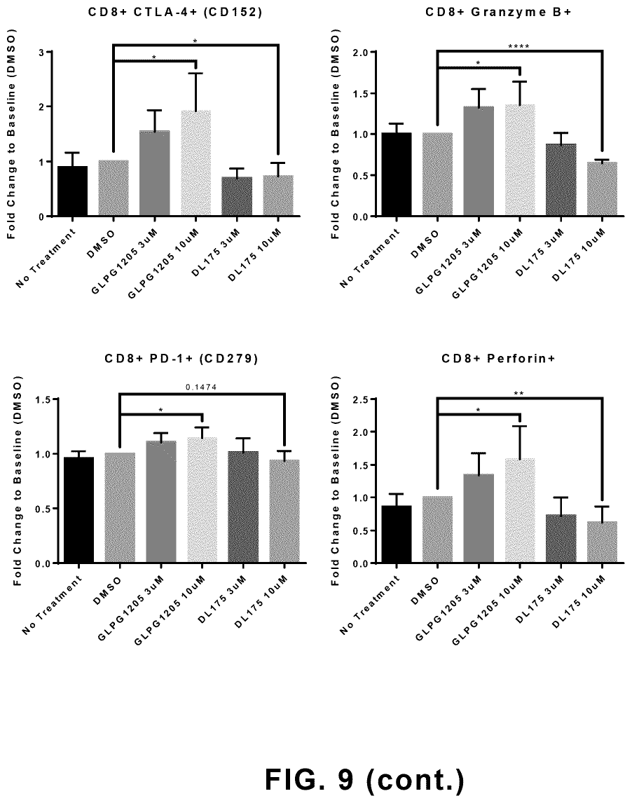

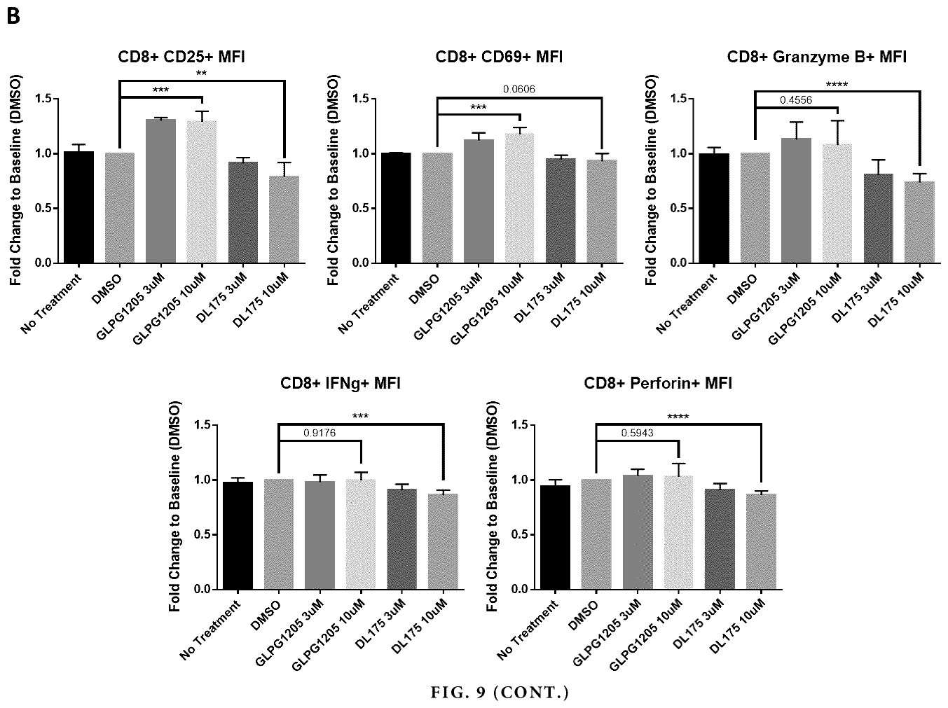

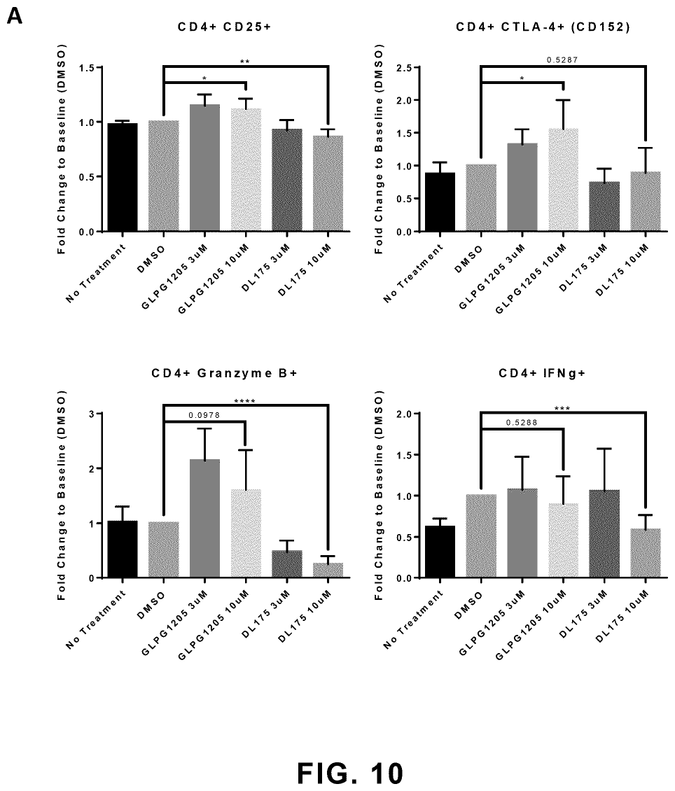

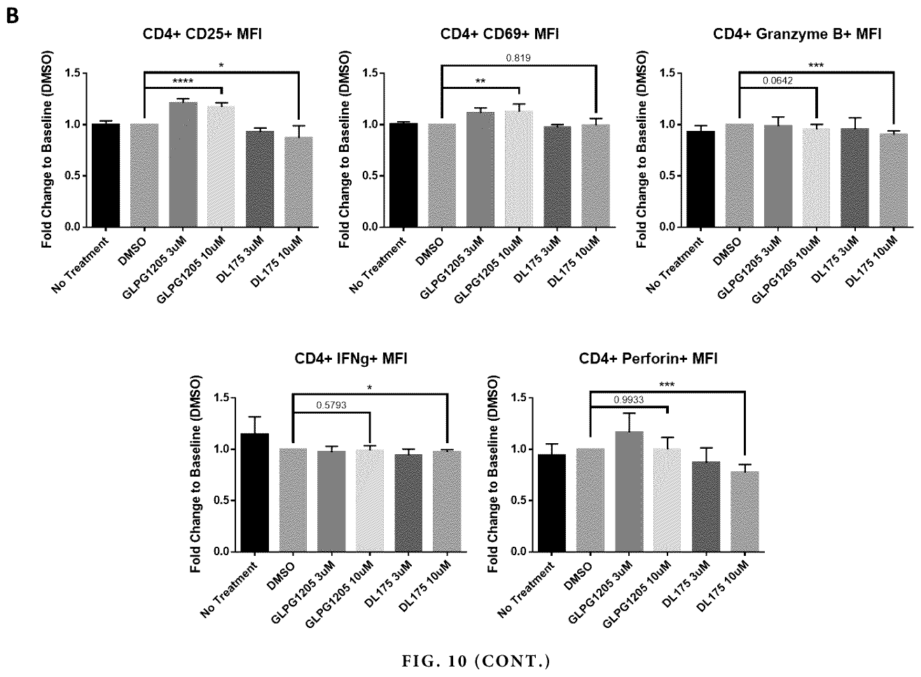

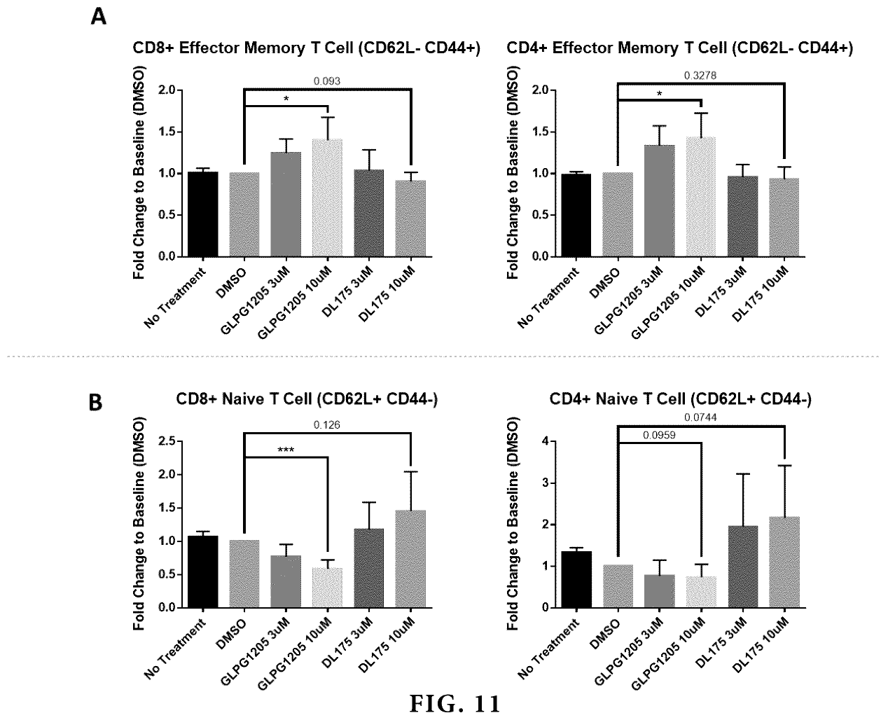

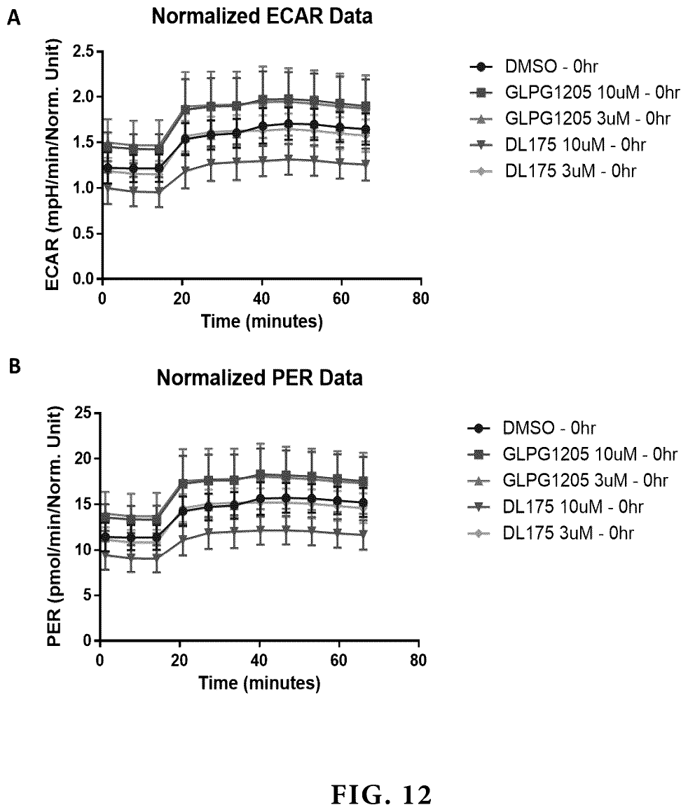

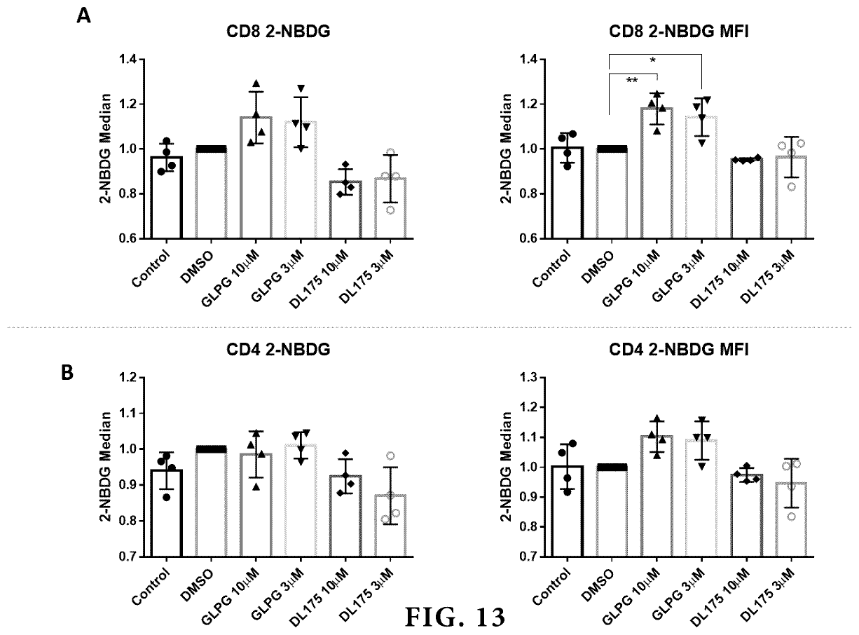

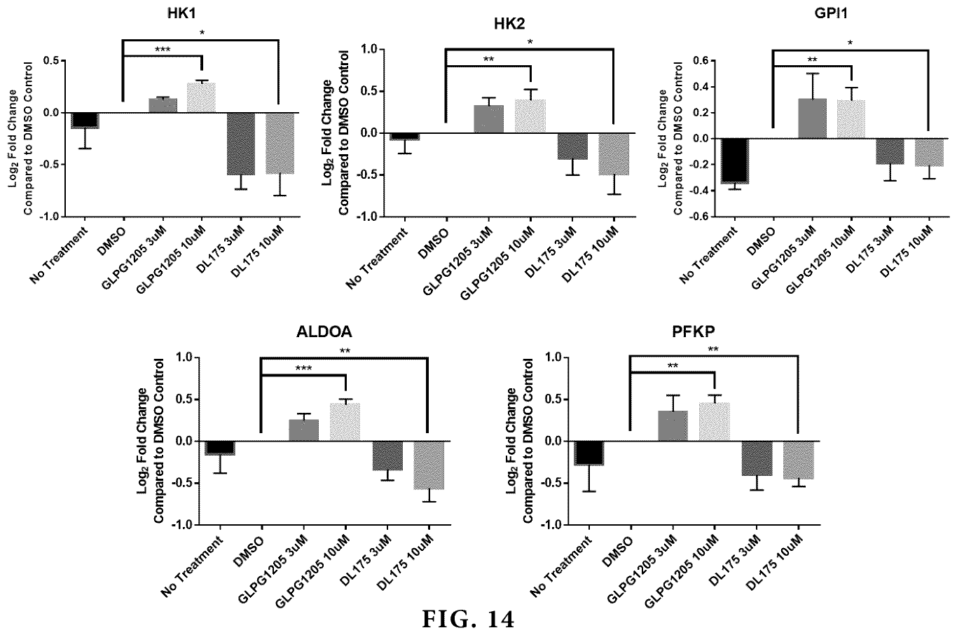

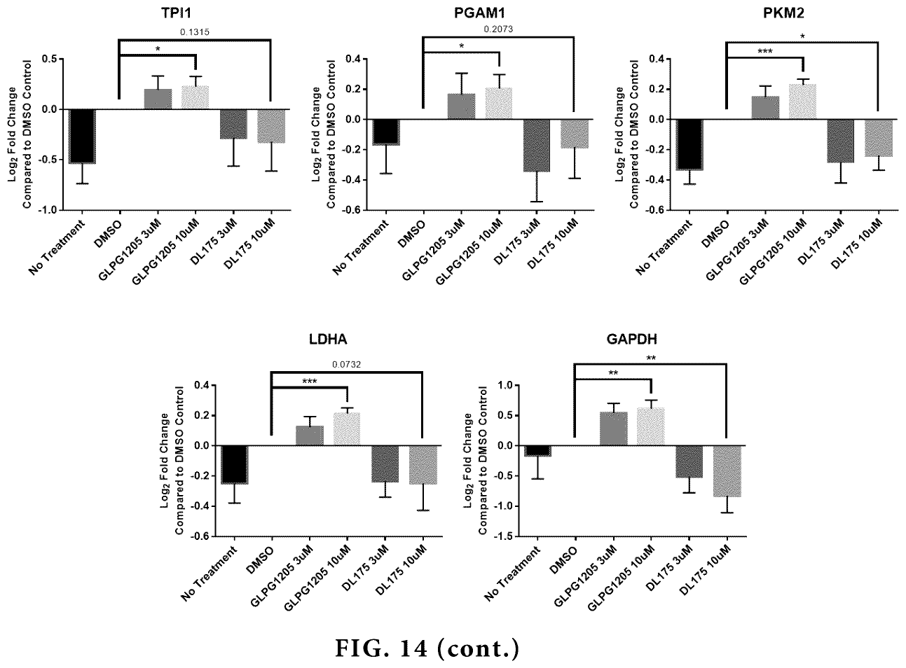

Beyond cytokines highly effective for T cell activation and proliferation, we investigated if any other inflammatory cytokines and chemokines were affected by GPR84 regulation. In the same multiplex panel, we found that a large number of additional cytokines were directly affected by GPR84 modulation. G-CSF, a cytokine which promotes granulocyte production significantly increased 16.64% when treated with 3 μM antagonist GLPG1205 and by 26.52% at the 10 μM dose compared to DMSO control. In contrast, there was no significant reduction in G-CSF at either DL175 agonist dose. (FIG. 20 Panel F) We next examined the effect of GPR84 modulation on GM-CSF, a cytokine which promotes both granulocyte and macrophage production. With this cytokine, we observed a significant 32.93% increase with 3 μM antagonist GLPG1205, and a significant 123.81% decrease with 10 μM agonist DL175. (FIG. 20 Panel G) IL-1P, a pyrogenic cytokine involved in proliferation and differentiation, significantly increased by 20.06% in the 3 μM GLPG1205 treated group. This was in contrast to the significant reductions of 12.99% at 3 M and 35.76% at 10 μM DL175 agonist treatment. (FIG. 20 Panel H) We next examined the cytokine IP-10, or CXCL10, a chemokine involved in promoting various immune cell migration and adhesion. We found that both GLPG1205 antagonist concentrations significantly increased secretion compared to DMSO control (3 M, 28.52%; 10 μM, 73.65%). In opposite we found that IP-10 significantly reduced secretion by 19.05% in the 10 μM DL175 agonist treatment group. (FIG. 20 Panel I) Subsequently we studied the effect of IL-6, a pleiotropic cytokine which can elicit pyrogenic effects, induce the maturation of pro-inflammatory immune cells, while also inducing the differentiation of immunosuppressive MDSCs. We found that CD3 T cells treated with 3 μM and 10 μM GLPG1205 significantly increased IL-6 by 30.71% and 28.38% respectively. Surprisingly, we did not notice a significant effect after treatment with agonist DL175. (FIG. 9J) We next studied GPR84 modulation on Eotaxin secretion. Eotaxin, or CCL11, is a eosinophil-specific chemokine. We found that treatment with GLPG1205 significantly increased Eotaxin levels compared to DMSO control (3 M, 19.87%; 10 μM, 16.57%). In contrast, Eoxtain levels significantly reduced by 22.49% at the 10 μM DL175 agonist dose. (FIG. 20 Panel K) We subsequently looked at the effect on LIF production, a cytokine which induces hematopoietic differentiation, and found that GPR84 modulation significantly changes production at all doses measured. When CD3 T cells were treated with GPR84 antagonist GLPG1205, LIF significantly increased by 41.71% at 3 M and 52.21% at 10 μM. In opposition, treatment with agonist DL175 significantly decreased LIF levels by 42.55% at 3 M and 98.85% at 10 μM concentrations. (FIG. 20 Panel L) The next cytokine studied was LIX, or CXCL5, a chemokine involved in neutrophil chemotaxis and recruitment. For this cytokine, only 10 M GLPG1205 was found to elicit a significant increase of 20.94%. (FIG. 20 Panel M) We subsequently examined IL-10, a cytokine known to have both inflammatory and anti-inflammatory properties. Known to induce immunosuppression through reduced Th1 cytokine secretion and NFκB activity, this cytokine also promotes B cell proliferation and survival. We found that in all conditions, IL-10 was significantly changed compared to the DMSO control. 3 μM and 10 μM GLPG1205 antagonist increased IL-10 by 52.24% and 58.46% respectively. In opposite 3 μM and 10 μM DL175 agonist reduced IL-10 by 51.73% and 175.14%. (FIG. 20 Panel N) Continuing, we studied GPR84 modulation on IL-4 secretion, a cytokine secreted by activated T cells. IL-4 acts as an immune regulator by dampening the effects of inflammatory cytokines and promoting tissue repair. At 3 μM GLPG1205, IL-4 secretion significantly increased by 26.55%, and did not elicit an effect at 10OM. With DL175 agonist treatment, secretion significantly reduced by 53.59% at 3 M and 121.42% at the 10 μM concentration. (FIG. 20 Panel O) The next cytokine examined was IL-3, a cytokine involved in proliferation and differentiation of immune cells. We found that modulation of GPR84 receptor significantly affected IL-3 at all tested doses. Treatment with 3 μM GLPG1205 increased cytokine secretion by 31.91%. This was increased to 38.29% in the 10 μM treatment group. IL-3 secretion subsequently reduced by 47.79% after 3 M and 130.6% after 10 M DL175 treatment. (FIG. 20 Panel P) Continuing, we next looked at how GPR84 modulation affected MCP-1 secretion. MCP-1, or CCL2, is a chemokine involved in monocytes and basophil chemotaxis and recruitment. For this cytokine we only observed a significant changed in the 10 μM GLPG1205 antagonist group, with an increase of 21.29% compared to DMSO control. (FIG. 20 Panel Q) Following MCP-1, we looked at GPR84's modulatory effects on VEGF, a cytokine associated with promoting neovascularization. For VEGF, we found that drug conditions elicited significant changes. Treatment with 3 μM GLPG1205 antagonist increased VEGF by 51.99% and this was further increased to 71.18% at the 10 μM treatment dose. In contrast, VEGF reduced by 71.22% with 3 μM DL175 agonist treatment, and further enhanced to 145.73% reduction at the higher 10 μM dose. (FIG. 20 Panel R) The next cytokine examined was KC, or CXCL1, a chemokine involved in neutrophil chemotaxis and recruitment. Here, we saw that T cells treated with 3 μM and 10 μM GLPG1205 significantly increased KC concentrations by 19.27% and 25.62%. We found no effect at 3 M DL175, but a significant 33.26% reduction at 10 μM. (FIG. 20 Panel S) Finally, the last chemokine to provide significant changes with GPR84 regulation was RANTES. RANTES, or CCL5, is a chemokine involved in monocytes, memory Th cells, and eosinophil chemotaxis and recruitment. For this cytokine, we observed a significant 29.8% increase after treatment with 10 μM antagonist GLPG1205. We observed no other significant changes in the other conditions. (FIG. 20 Panel T)

### 4.1.7 GPR84 Receptor Modulation Regulates CD8 T Cell Differentiation

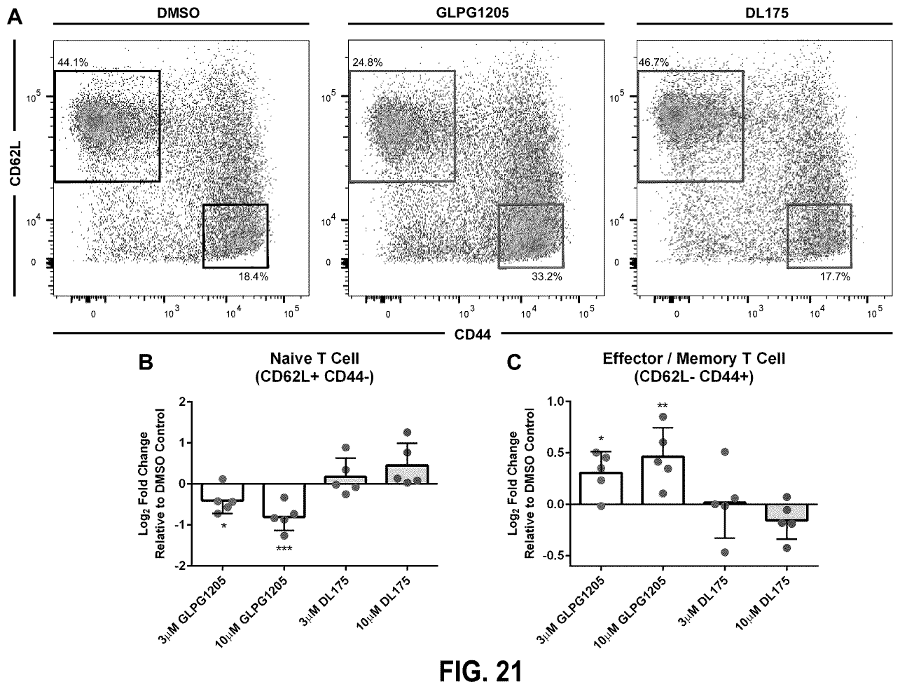

There is ample literature evidence to demonstrate that upregulated markers and cytokines secreted play an important role in T cells differentiation, activation, and proliferation. We showed that GPR84 regulation can affect activation marker expression and inflammatory cytokine production, leading to a functionally more cytotoxic CD8 T cell. However, the ability for GPR84 to modulate CD8 T cell differentiation remained unexplored. To address this, we differentiated naïve CD8 T cells with anti-CD3/anti-CD28 in the presence or absence of GPR84 receptor antagonist GLPG1205 or agonist DL175. We identified naïve T cells by CD62L+, CD44− and effector or memory CD8 T cells by CD62L−, CD44+. (FIG. 21 Panel A) We found that when T cells were treated with 3 μM or 10 μM GLPG1205, the naïve CD8 T cell compartment reduced by an average of 32.54% and 74.69% respectively compared to DMSO control. In contrast, treatment with 3 μM and 10 μM DL175 slightly increased the naïve compartment, but we did not find this change to be significant. (FIG. 21 Panel B) We next wanted to understand if this change in the naïve CD8 T cell compartment can subsequently lead to an increase or decrease in the effector/memory CD8 T cell compartment. As expected, when we differentiated naïve T cells in the presence of 3 μM or 10 μM GLPG1205, the effector/memory compartment significantly increased by an average 23.57% and 37.93% compared to DMSO control. Similarly, we found no significant reductions in the effector/memory CD8 T cell fraction at either DL175 concentrations. (FIG. 21 Panel C)

GPR84 receptor modulation regulates CD4 T cell differentiation

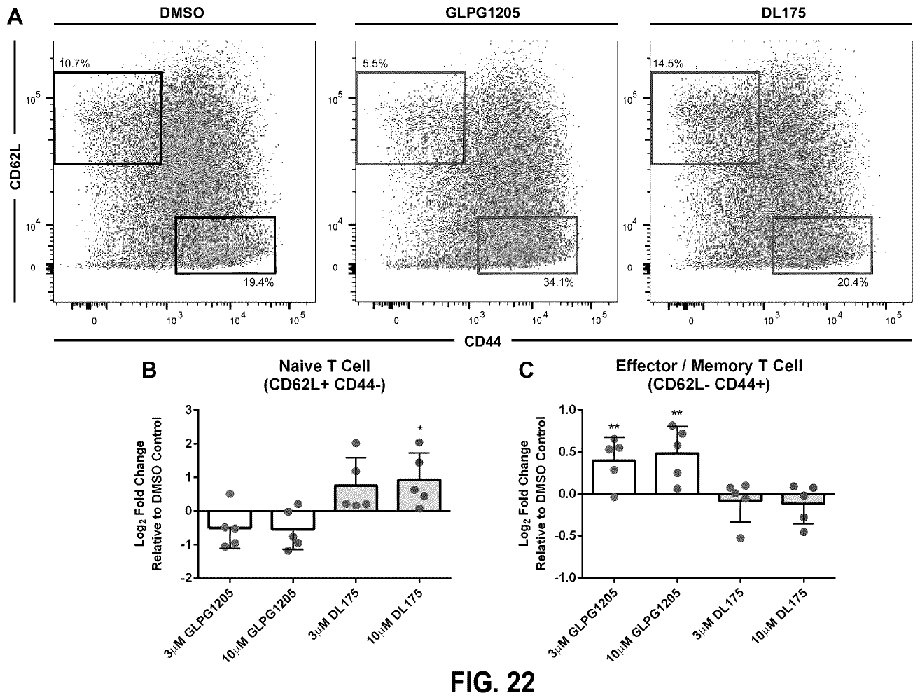

Like CD8, we determined how GPR84 modulation affected T cell differentiation in the CD4 T cell compartment. We identified naïve (CD62L+, CD44−) and effector/memory (CD62L−, CD44+) CD4 T cells in the same manner as the CD8 compartment. (FIG. 22 Panel A) When we examined the naïve CD4 T cell compartment after GLPG1205 antagonist treatment, we observed a reduction similar to the CD8 T cells, but this trend was not found to be significant (3 M, 41.57%; 10 μM, 45.61%). Similarly, we observed a trending, but non-significant 68.83% increase in the naïve CD4 compartment after 3 M DL175 agonist treatment. However, we did see a significant 89.97% increase in naïve CD4 T cell compartment at the higher 10 μM dose. (FIG. 22 Panel B) In turn, we wanted to determine if these trends in the naïve compartment can lead to significant changes in the effector/memory compartment. In these studies, we did see a significant increase in effector/memory T cells with both 3 μM and 10 μM GLPG1205 antagonist treatments (3 M, 31.55%; 10 μM, 39.79%) compared to DMSO control. Like the CD8 population, when we treated CD4 T cells with GPR84 agonist DL175, we did not observe a significant change in the effector/memory phenotype. (FIG. 22 Panel C)

GPR84 receptor knockout by CRISPR-Cas9 enhances CD8 T cell cytotoxic capacity

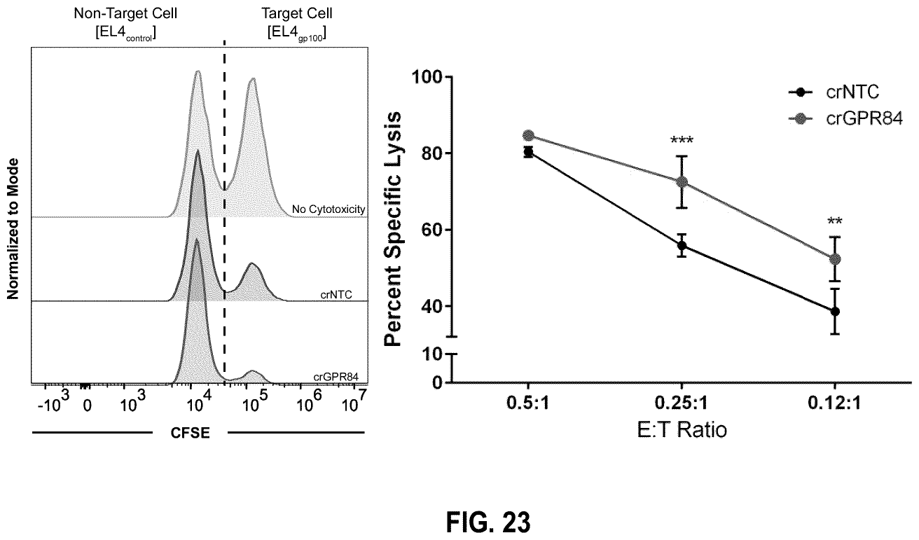

To test that our enhanced cytotoxic capacity with GPR84 receptor antagonism GLPG1205 was not due to a pharmacological off-target effect, we compared the cytotoxic capacity of CRISPR-Cas9 GPR84 knockout primary pmel-I CD8 T cells (crGPR84) compared to a negative no target control (crNTC). Using gp100 pulsed EL4 tumor cells as the target cell, removal of GPR84 by CRISPR-Cas9 significantly improved the cytotoxic capacity. Referencing the representative FIG. 23, we did not find a significant difference in cytotoxicity at the highest, 0.5:1 effector to target ratio. However, we found significant changes at the 0.25:1 ratio, with GPR84 knockout inducing a 72.5% specific lysis compared to 55.9% in the no target control. Furthermore, we observed preservation of the significant cytotoxic enhancement at the lower 0.12:1 effector to target ratio, with 52.3% specific lysis in the crGPR84 T cells compared to 38.6% in the crNTC CD8 T cells.

### The Effect of GPR84 Modulation on the Metabolic Profile of Immune Cells

### GPR84 Receptor Modulation Metabolically Reprograms CD8 T Cells

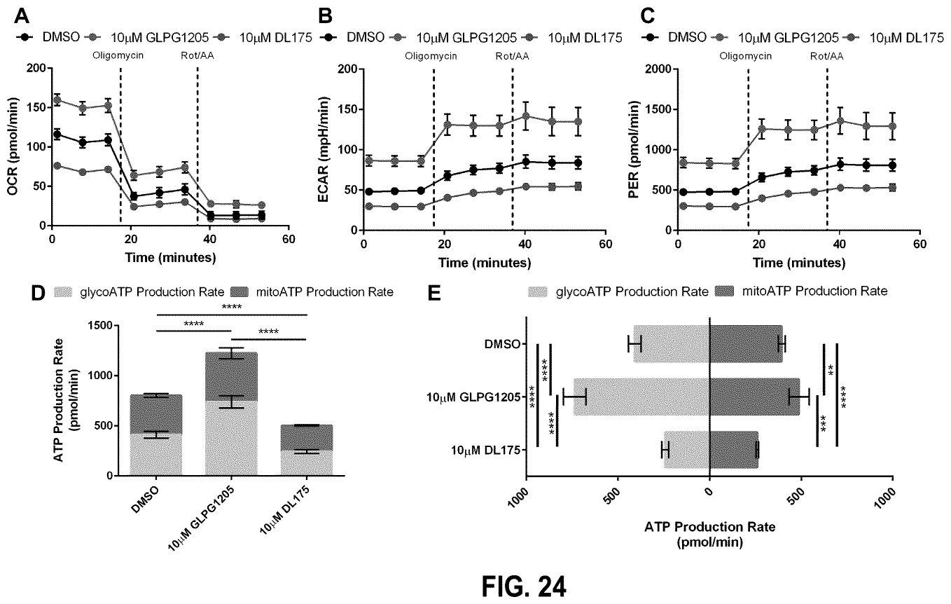

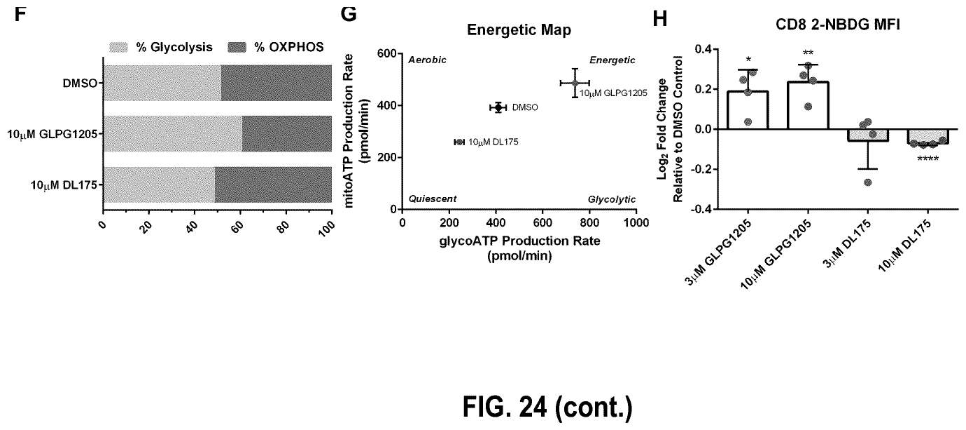

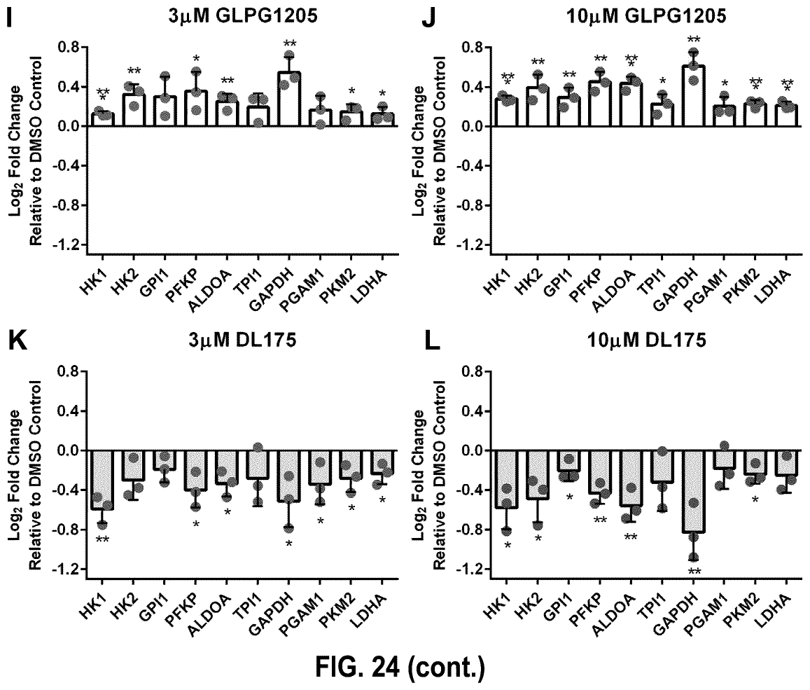

Using the research demonstrating GPR84's role in metabolic homeostasis as evidence, we explored if these changes in T cell activity and function can be a result from alterations in effector T cell metabolism. To do this, we used seahorse to measure the oxygen consumption rate (OCR), extracellular acidification rate (ECAR), and proton efflux rate (PER). We found that treated CD8 T cells with antagonist GLPG1205 raises the overall OCR, ECAR, and PER. In contrast, treatment with DL175 agonist had an opposite effect with overall reductions. (FIG. 24 Panels A-C) Furthermore, these readouts can provide the information to determine the overall ATP production rate, and the amount contributed by glycolysis or mitochondrial OXPHOS. Using this strategy, we had at least two goals, the first to understand how GPR84 modulation affected overall metabolic activity and second, to dissect the percent reliance on either glycolysis or OXPHOS for ATP generation. Referencing the representative FIG. 24 Panel D, we found that GPR84 modulation significantly affected the total ATP production rate. We found that DMSO control ATP production rate to be 816.19 pmol/min. When adding the antagonist GLGP1205, the production rate increased to 1224.27 pmol/min, and when adding the agonist DL175, this rate decreased to 502.64 pmol/min.

After determining that GPR84 did affect the total ATP production, the next step was to stratify the total ATP generation by either glycolysis or mitochondrial OXPHOS. After separating these values, we found that both ATP generating processes were significantly affected by GPR84 modulation. Use of the GPR84 antagonist GLPG1205 significantly increased ATP production through glycolysis compared to DMSO control (737.62 vs. 409.61 pmol/min). Additionally, a significant increase was also observed in ATP production through mitochondrial OXPHOS (486.75 vs. 392.74 pmol/min). The use of the GPR84 agonist DL175 had an opposite effect with a significant reduction in ATP rate through glycolysis (242.85 vs. 409.61 pmol/min) and mitochondrial OXPHOS (259.85 vs. 392.74 pmol/min) compared the DMSO baseline. (FIG. 24 Panel E) After determining that GPR84 modulation did change the rate of ATP production, we elucidated if modulation affected which ATP generating process the CD8 T cells more favorably relied on. To address this, the rate of ATP production was broken down by percent generated by glycolysis and OXPHOS. The split with DMSO control was found to be 51% glycolysis and 49% OXPHOS. Interestingly, by adding antagonist GLPG1205, this percentage changed to 60.3% glycolysis and 39.7% OXPHOS, indicating a stronger reliance on glycolysis for ATP production. Finally, we did not see dramatic changes after DL175 agonist addition, with ATP production relying 48.25% on glycolysis and 51.75% on OXPHOS. (FIG. 24 Panel F) After observing these changes in the ATP production landscape, we were interested if the changes can be associated with a change in an energetic phenotype. To do this, we used an energetic map, which generates an energetic phenotype based on the amount of ATP produced through either glycolysis or through the mitochondria. When mapped, DMSO plotted in the center of the map, indicating the baseline activity state. CD8 T cell treatment with antagonist GLPG1205 caused cells to exhibit an energetic phenotype, while CD8 T cells treated with agonist DL175 exhibited a quiescent phenotype. (FIG. 24 Panel G)

Now knowing that CD8 T cells after treatment with antagonist GLPG1205 relied on glycolysis as their mode of ATP generation, we wanted to see if there was an increase in the uptake of glucose from the surrounding environment. Using the fluorescent glucose analog, 2-NBDG, we found that T cells treated with the antagonist GLPG1205 led to a significant uptake at both the 3 μM (0.19 Log₂ fold change, or 13.92%) and 10 μM (0.24 Log₂ fold change, or 17.74%) concentrations compared to DMSO control. Use of the agonist DL175 at 3 μM did not yield a significant change, but at the higher 10 μM dose, 2-NBDG uptake was significantly reduced by −0.07 Log₂ fold change, or 5.01% compared to DMSO control. (FIG. 24 Panel H)

To link the increased uptake in glucose with the identified increase in glycolysis generated ATP, we looked at the relative gene expression fold-change of multiple enzymes in glycolytic pathway. We then compared the effect of GPR84 modulation on gene expression to the DMSO treated control. Referencing FIG. 24 Panels I-J, we found that 3 M and 10 M antagonist GLGP1205 treated CD8 T cells elicited a significant dose-dependent increase in the gene expression compared to DMSO control. In reverse, CD8 T cells treated with 3 μM and 10 μM DL175 agonist had a significant dose-dependent decrease in gene expression compared to DMSO control. (FIG. 24 Panels K-L)

### GPR84 Receptor Modulation Metabolically Reprograms BM-MDSCs

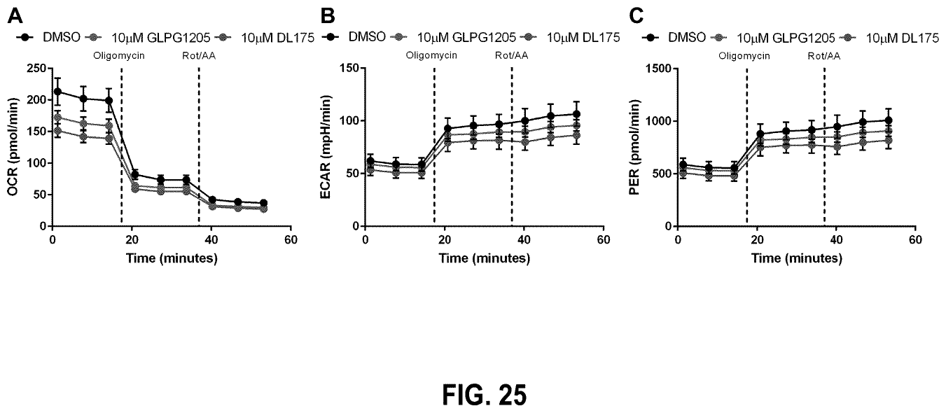

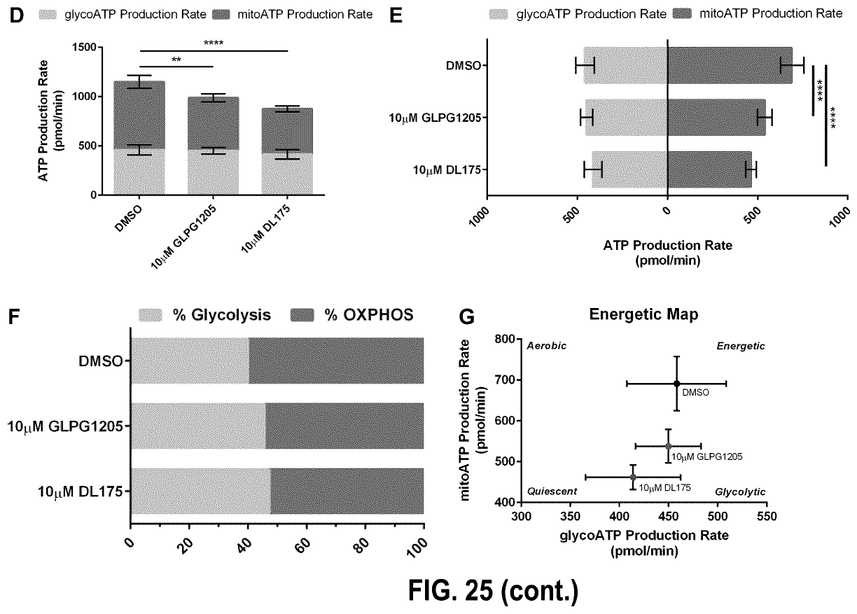

Weshow that GPR84 receptor can modulate CD8 T cell metabolism. Building on this finding, we also wanted to see if our observed changes in BM-MDSCs activity was also attributed to metabolic modulation. Like CD8 T cells, we addressed these questions using seahorse to measure the oxygen consumption rate (OCR), extracellular acidification rate (ECAR), and proton efflux rate (PER). Surprisingly, we found a different metabolic profile compared to the previously examined CD8 T cells. In this case, we found that both GLPG1205 and DL175 lead to an overall reduction in OCR. Subsequently, we did not observe any changes with either drug on ECAR and PER. (FIG. 25 Panels A-C) Using the OCR, ECAR, and PER values, we determined the overall ATP production rate, and the amount contributed by glycolysis or mitochondrial OXPHOS. Like the CD8 T cells, we used the ATP rate assay for at least two objectives, to first to understand how GPR84 modulation affected overall metabolic activity and second, to dissect the percent reliance on either glycolysis or OXPHOS for ATP generation. Referencing FIG. 25 PanelD, we found that both GLPG1205 and DL175 significantly affected the total BM-MDSC ATP production rate. We measured DMSO control ATP production rate to be 1149.1 pmol/min. When adding the antagonist GLPG1205, the production rate significantly decreased to 987.48 pmol/min, and when adding the agonist DL175, the production rate also significantly decreased to 875.55 pmol/min.

After determining that GPR84 did affect the total ATP production, we wanted to know the amount of ATP generated from either glycolysis or mitochondrial OXPHOS. To do this, we stratified the ATP production rate and interestingly found that the observed changes in ATP production were only due to an effect on mitochondrial OXPHOS. When examining the rate of ATP production through mitochondrial OXPHOS, we found significant reductions with GPR84 antagonist GLPG1205 (537.83 vs. 690.95 pmol/min) and GPR84 agonist DL175 (461.66 vs. 690.95 pmol/min) treatment compared to DMSO control. (FIG. 25 Panel E) Like with CD8s, after determining that GPR84 modulation did change the rate of ATP production, we wanted to elucidate if GPR84 modulation affected which ATP generating process BM-MDSCs more favorably relied on. To address this, we broke the rate of ATP production down by percent generated by glycolysis and by OXPHOS. We found that in BM-MDSCs treated with DMSO, the ATP generation was split by 39.85% glycolysis and 60.15% OXPHOS. When we added GLPG1205 to BM-MDSC, this percentage changed to 45.55% glycolysis, and 54.45% OXPHOS, suggesting a reduction in mitochondrial activity. Next, we saw similar changes when DL175 was added to BM-MDSCs, with ATP production relying 47.16% on glycolysis, and 52.84% on OXPHOS. (FIG. 25 Panel F)

We wanted to see if these changes in the ATP production landscape associated with a change in the energetic phenotype. Using an energetic map, we found that DMSO plotted in the upper right corner, indicating the baseline activity state. Treatment of BM-MDSCs with antagonist GLPG1205 and agonist DL175 exhibited movement from an aerobic-predominant energetic phenotype to a more glycolytic-predominant quiescent phenotype. (FIG. 25 Panel G)

GPR84 receptor modulation of CD8 T cells reveals differentiation, metabolic and effector genomic changes by single-cell RNA sequencing

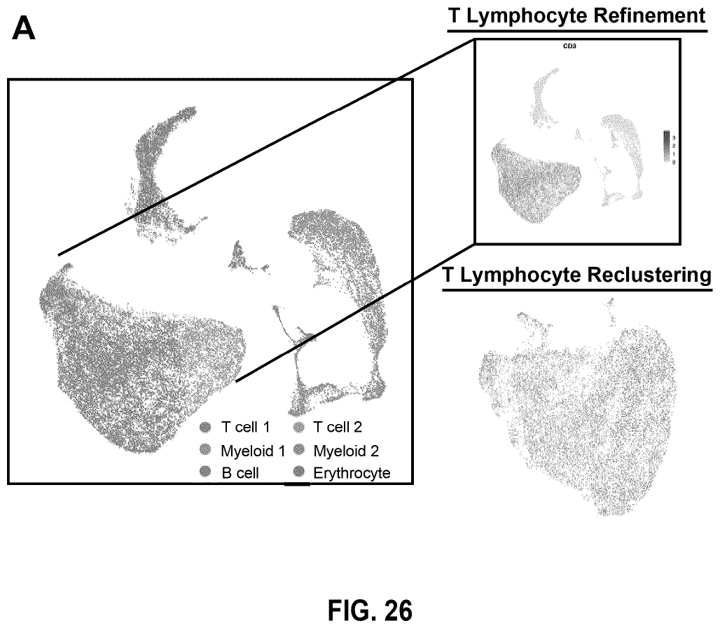

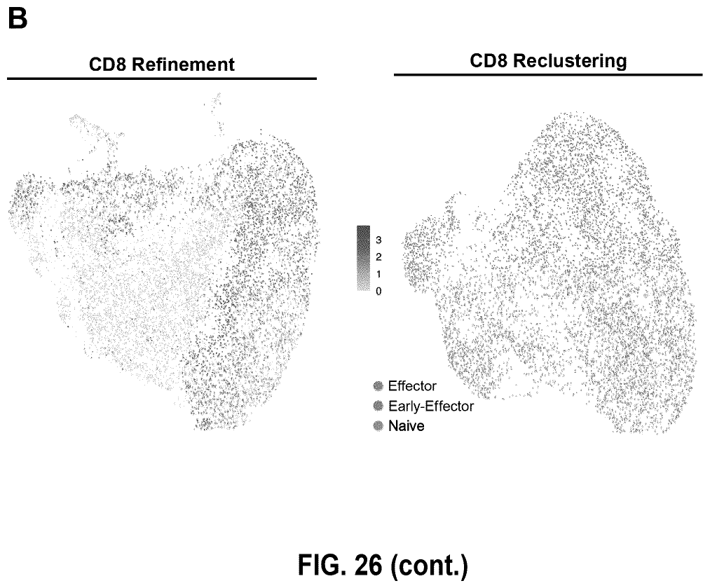

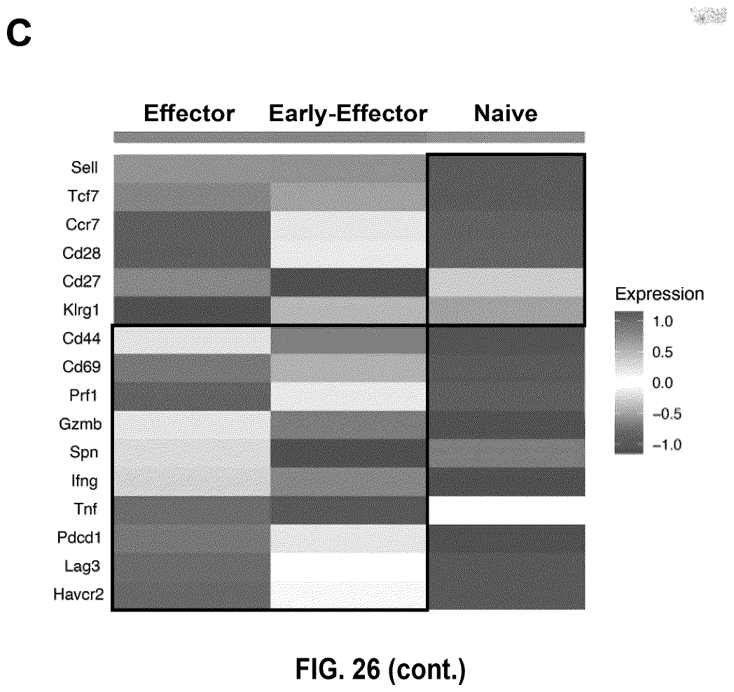

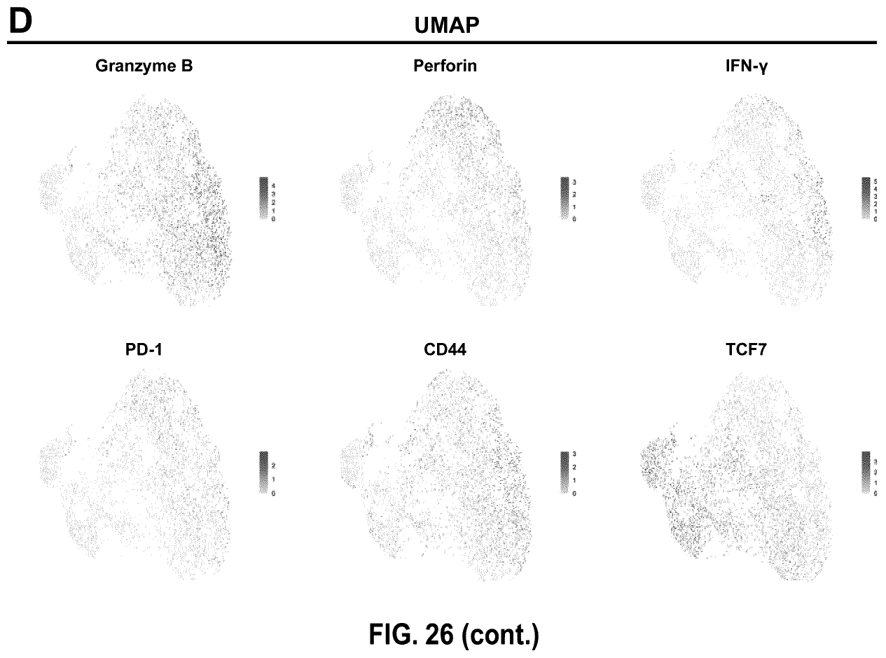

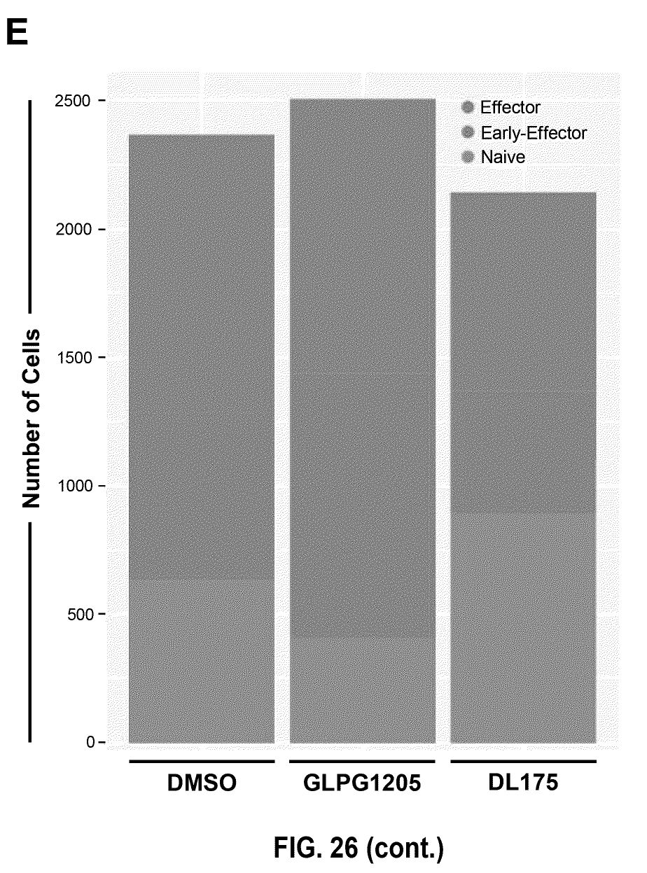

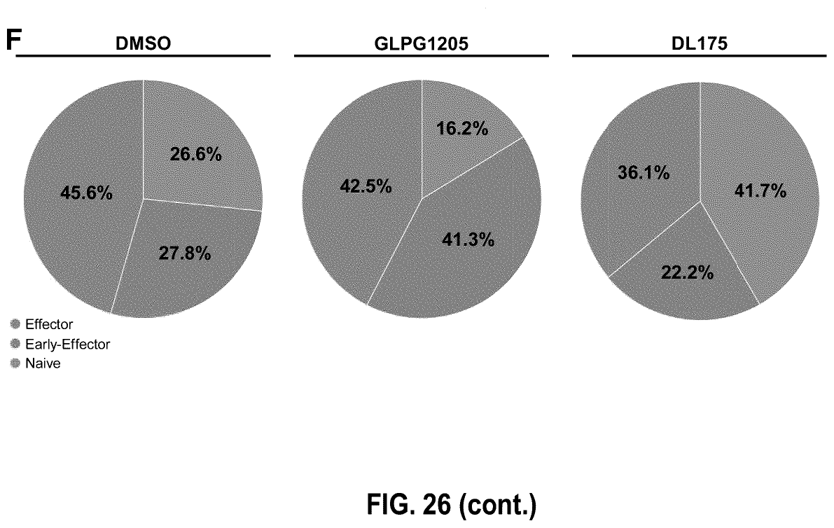

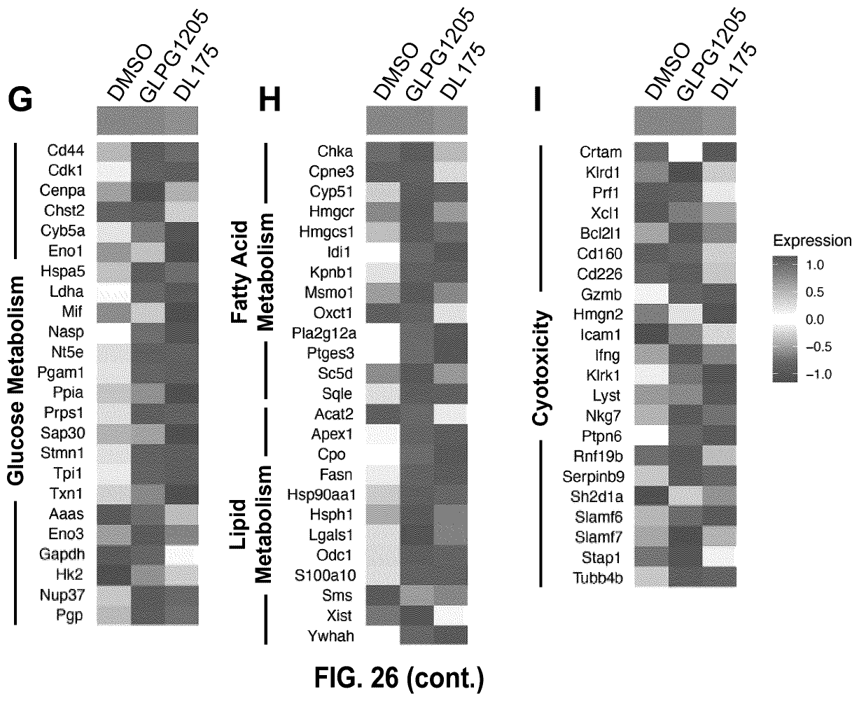

Building on the evidence that GPR84 regulation is a metabolic regulator of CD8 T cells, we wanted to have a understanding of how GPR84 receptor modulation alters the CD8 genomic landscape. To do this, we analyzed a heterogenous mixture of immune cells treated with GLPG1205 antagonist, DL175 agonist, or DMSO control by single-cell RNA sequencing (scRNA-seq). First, cells underwent an unsupervised clustering and graphed on a UMAP plot. (FIG. 26 Panel A) We then identified the cytotoxic CD8 T cell compartment by conducting a CD3 followed by a CD8 refinement and reclustering. (FIG. 26 Panel B) CD8 reclustering allowed us to identify three genetically unique compartments. We identified the first population as an effector CD8 T cell, comprising of high expression of associated cytotoxic genes, and low expression of naïve CD8 T cell genes. The second was characterized as an early-effector CD8 T cell, expressing moderate intensity in cytotoxic genes, and low expression in naïve genes. We identified the final population as a naïve CD8 T cell, comprising of a low cytotoxic and elevated naïve gene expression. (FIG. 26 Panel C). We created UMAP plots of granzyme B, perform, IFNγ, PD-1, and CD44 to demonstrate a convergence at the effector and early-effector CD8 clusters. As expected, TCF7, a gene associated with an immature CD8 phenotype, was found to converge in the identified naïve compartment. (FIG. 26 Panel D) We next plotted the total number of CD8 T cells by treatment. As expected, we saw an increase in total CD8 in the 10 μM GLPG1205 antagonist condition and reduced numbers in the 10 μM DL175 agonist condition compared to the DMSO control. (FIG. 26 Panel E) Interestingly, when we subdivided the total CD8 by our three identified subclustered, we found stark differences between groups. In the DMSO treatment, we identified 45.6% as effector, 27.8% as early-effector, and 26.6% as naïve T cells. When CD8 T cells were treated with 10 μM of the antagonist GLPG1205, these fractions changed to 42.5% effector, 41.3% early-effector, and 16.2% naïve. Lastly, after the addition of 10 M agonist DL175, the fractions changed to 36.1% effector, 22.2% early-effector, and 41.7% naïve. (FIG. 26 Panel F)

In addition, we wanted to see if the genetic profile of treated CD8 T cells can expand on the metabolic and cytotoxic changes elucidated. To do this, we generated a curated list of genes associated with glucose metabolism, fatty acid metabolism, lipid metabolism, and cytotoxicity. We then plotted the average gene expression as a heatmap. In all curated gene lists we found similar gene expression changes. The was a dramatic increase in gene expression in the 10 μM GLPG1205 antagonist group, and a subsequent reduction in the 10 μM DL175 agonist group. (FIG. 26 Panels G-I)

The effect of GPR84 modulation on anti-tumor immunity

Pre-treatment of adoptively transferred CD8 T cells with GPR84 antagonist GLPG1205 confers an enhanced in vivo anti-tumor response

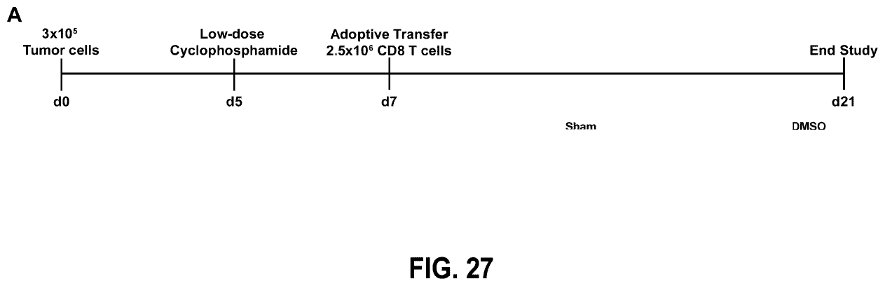

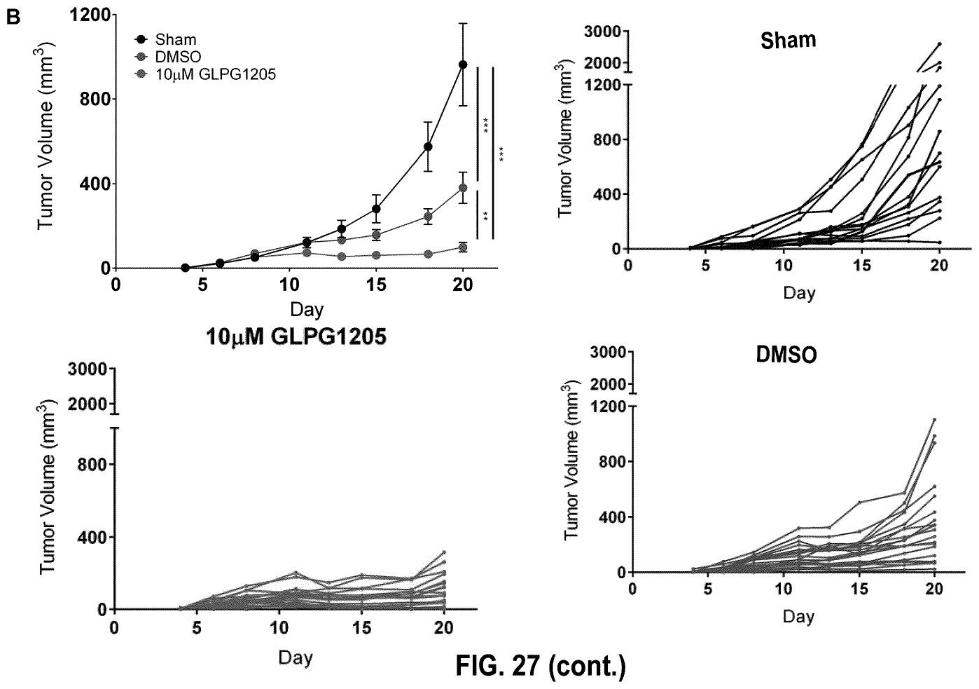

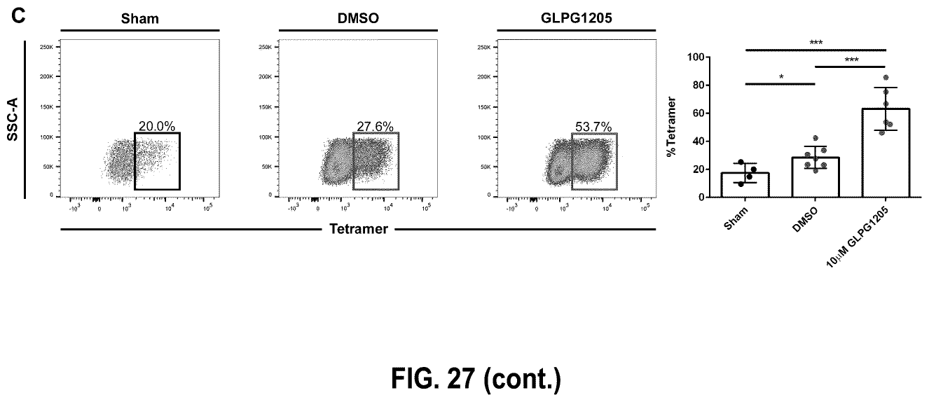

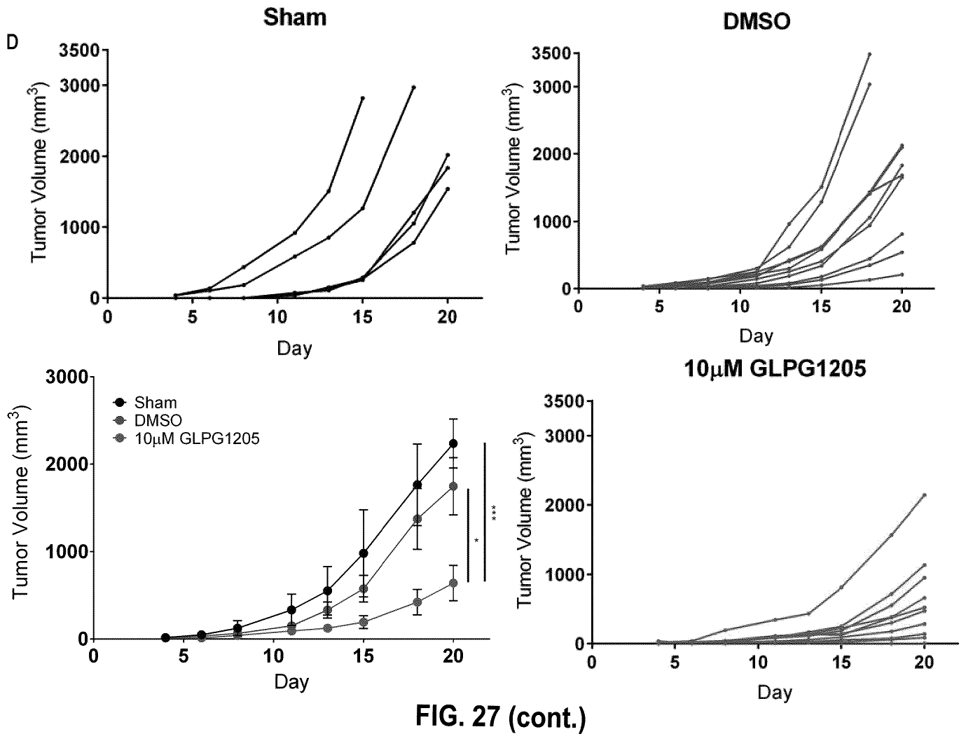

After determining the increased activity in CD8 T cells after GPR84 antagonism with GLPG1205, we wanted to see if these changes can have any anti-tumor implications. To examine this, we used the OT-I and pmel-I adoptive immunotherapy models. Mice were inoculated in the flank with B16-OVA for the OT-I, or B16 for the pmel-I transfers. We then lymphodepleted mice using low-dose cyclophosphamide and then adoptively transferred activated CD8 T cells pre-treated with either 10 μM GLPG1205 or DMSO. Starting from day four, tumor volumes were measured for the study duration. (FIG. 27 Panel A) Using the OT-I system, we found that adoptive transferring DMSO pre-treated OT-I elicited a significant anti-tumor response compared to a sham adoptive transfer control. Interestingly, activating OT-I CD8 T cells in the presence of 10 μM GLPG1205 significantly enhanced this anti-tumor effect. (FIG. 27 Panel B) We also wanted to determine the OVA-specific fraction of CD8 T cells entering into the tumor microenvironment. Using flow cytometry, in the sham group we found 20% of intratumoral CD8 T cells stained positive for SIINFEKL tetramer (SEQ ID NO: 1). This significantly increased to 27.6% in the DMSO pre-treated group. When we pre-treated T cells with 10 μM GLPG1205, the recovered tetramer positive CD8 T cells was 53.7%, significantly higher than the sham and DMSO treatment. (FIG. 27 Panel C) Finally, we wanted to demonstrate that this effect can also be observed in a different adoptive immunotherapy model. In this experiment, pmel-I CD8 T cells pre-treated with DMSO did not elicit a significant anti-tumor response compared to the sham adoptive transfer group. However, when pmel-I CD8 T cells were pre-treated with 10 μM GLPG1205, we once again observed a significant reduction in the tumor growth rate compared to both the sham and DMSO pre-treated groups. (FIG. 27 Panel D)

Pre-treatment of adoptively transferred CD8 T cells with GPR84 agonist DL175 confers an enhanced in vivo pro-tumor response

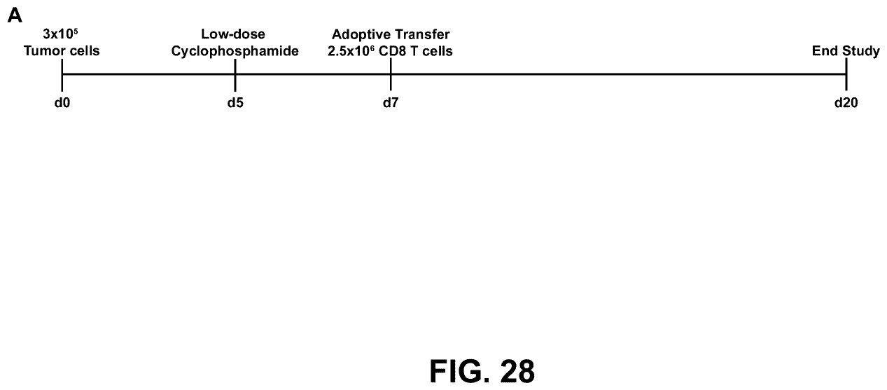

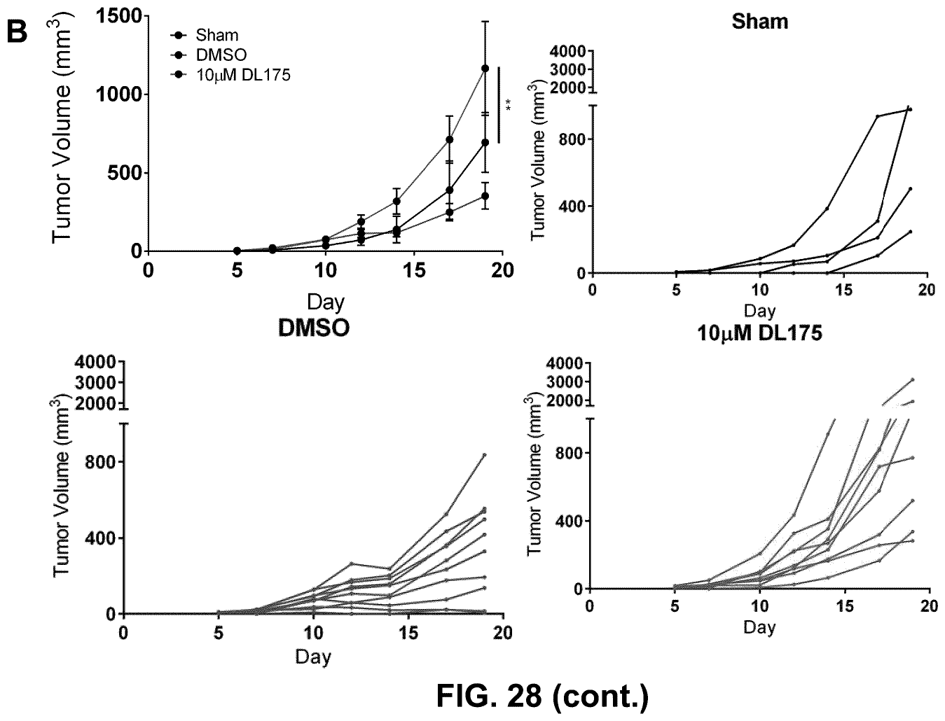

By determining the beneficial anti-tumor effects of pre-treating CD8 T cells with GPR84 antagonist GLPG1205, we were interested in understanding the effect on tumor growth after pre-treatment with GPR84 agonist DL175. Using the same experimental design, agonist pre-treated CD8 OT-I T cells were adoptively transferred into B16-OVA tumor bearing mice. (FIG. 17A). We found that there was a non-significant decline in anti-tumor effect between the sham and DMSO treated control. However, interestingly we found that DL175 agonist treatment worsened tumor progression and enhanced tumor growth significantly beyond the sham adoptive transfer control. (FIG. 28 Panel B)

In vivo titration of SIINFEKL (SEQ ID NO: 1) to achieve a suboptimal vaccination response

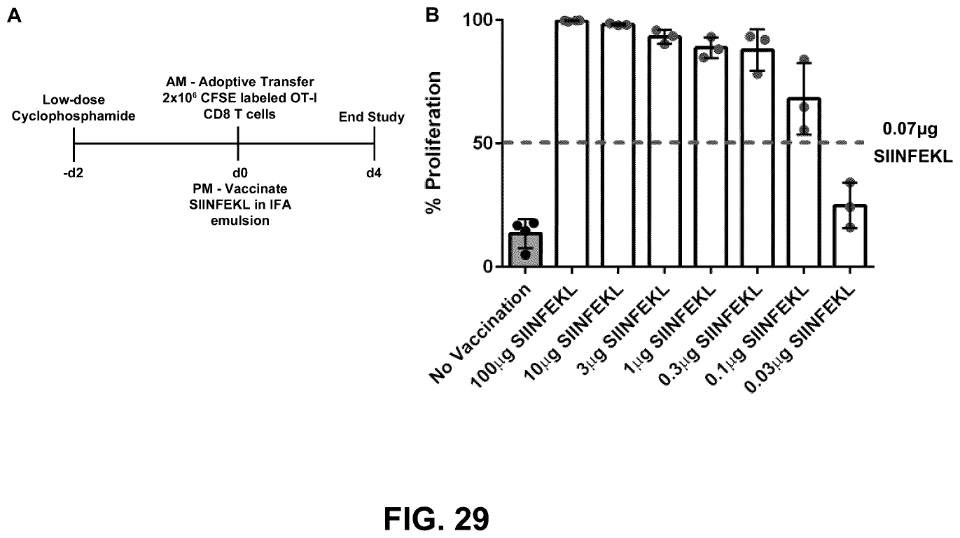

After we successfully determined that GLPG1205 can elicit an enhanced anti-tumor response through adoptive cellular transfer, we wanted to see if we can adapt GLPG1205 treatment for use in other anti-tumor therapies. Using data as evidence, we determined that GLPG1205 promotes the differentiation, proliferation and survival of CD8 T cells. Due to the strong observed effect that GLPG1205 antagonism has on enhancing T cell differentiation into the effector state, we selected a therapy that can align with these characteristics. Using these criteria, we selected a vaccine model, as exposure to antigen also induces reactive T cells to move from a naïve to an effector phenotype. To test GLPG1205's ability to enhance a vaccine response, we first needed to select and use a model which elicited a sub-optimal response. For these reasons, we selected a sub-optimal SIINFEKL (SEQ ID NO: 1) vaccination system with naïve OT-I adoptive transfer. Using this system allowed us to have a consistent number of adoptively transferred naïve OT-I CD8 T cells while simultaneously capable of titrating a well-characterized and responsive antigen. To identify a sub-optimal response, we reduced the amount of SIINFEKL antigen (SEQ ID NO: 1) used in the vaccine until we achieved a 50% OT-I proliferation response. To test this and optimize this system, we first lymphodepleted wild-type mice 48 hours prior to adoptive transfer. On the day of transfer, we intravenously injected 2×10⁶ naïve CD8 CFSE labeled OT-I T cells. We subsequently vaccinated mice with titrated doses of SIINFEKL (SEQ ID NO: 1) in incomplete Freund's adjuvant (IFA) until we observed a sub-optimal response. We used IFA to promote an immune response and mimic the adjuvant used in traditional clinical vaccines. After vaccination mice were monitored for the full 96-hour study duration. (FIG. 29 Panel A) At the end of the study, we isolated lymph nodes from vaccinated mice and determined the percent OT-I proliferation by CFSE dilution. As expected, SIINFEKL (SEQ ID NO: 1) was an extremely reactive antigen. OT-I proliferation neared 100% for the first five concentrations. Only when we dosed SIINFEKL (SEQ ID NO: 1) at 0.1 g did we begin to observe a reduction in CD8 OT-I T cell proliferation. We found that further reducing to 0.03 g elicited a response below 50%. This allowed us to select 0.07 g SIINFEKL (SEQ ID NO: 1) as our target sub-optimal vaccine dose, predicted to elicit a 50% response. (FIG. 29 Panel B)

GPR84 antagonist treatment improves in vivo vaccination response

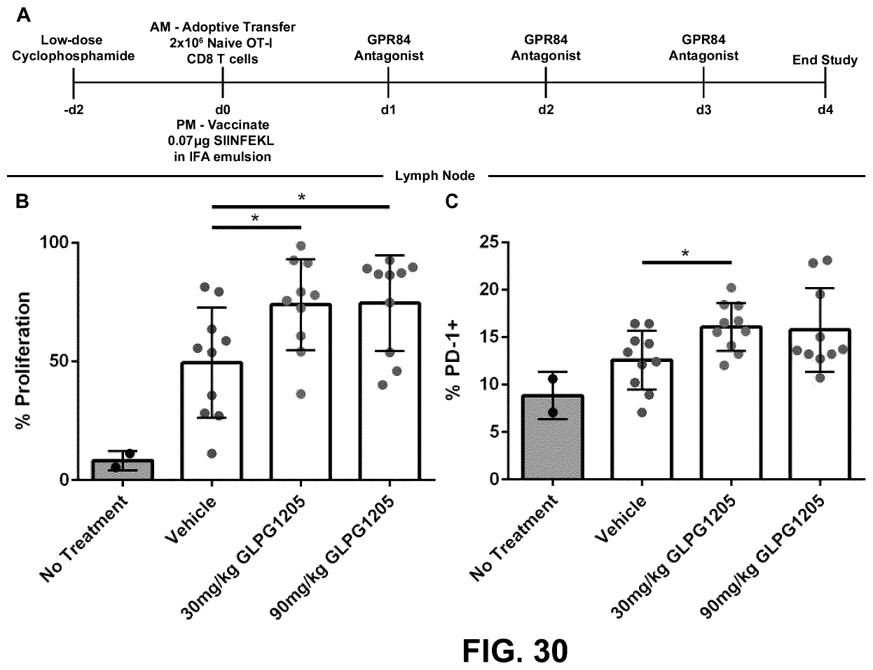

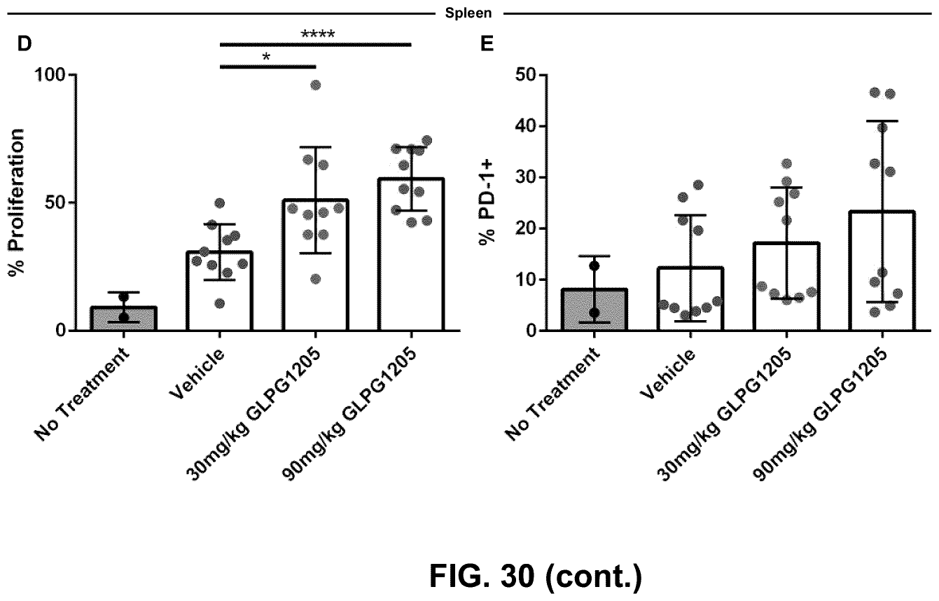

By finding the sup-optimal antigen dose for vaccination, we were able to test if we can improve the vaccination response with GPR84 antagonist GLPG1205. For these experiments, we lymphodepleted mice for 48 hours prior to adoptive transfer. We then adoptively transferred 2×10⁶ naïve CD8 CFSE labeled OT-I T cells followed by vaccination with 0.07 g SIINFEKL (SEQ ID NO: 1) in IFA. 24 hours after vaccination, we orally gavaged mice with either 30 mg/kg or 90 mg/kg GLPG1205 once daily for 3 days. We collected lymph node and spleens four days after transfer for analysis. (FIG. 30 Panel A) Our analysis found significant differences in adoptively transferred OT-I CD8 T cell proliferation and their levels of checkpoint protein PD-1 in lymph nodes and spleen of vaccinated and treated mice. In the lymph node compartment, we found that treating vaccinated mice with vehicle control methycellulose (MC) induced 49.37% OT-I CD8 T cell proliferation. When we treated vaccinated mice with 30 mg/kg GLPG1205, proliferation significantly increased to 73.84%. We observed a similar significant increase at the higher 90 mg/kg dose, with transferred T cells proliferating 74.56%. (FIG. 30 Panel B) Of these adoptively transferred cells, 12.58% expressed PD-1 in the MC vehicle control group. Like proliferation, PD-1 levels significantly increased to 16.05% after treatment with 30 mg/kg GLPG1205. However, increasing the dose to 90 mg/kg resulted in a non-significant increase to 15.75%. (FIG. 30 Panel C) Looking in the splenic compartment, we found a 30.68% basal level of proliferation after vaccination and MC treatment. Similar to the lymph nodes, mice treated with 30 mg/kg GLPG1205 induced a significant increase in proliferation to 50.99%. Likewise, we saw T cell proliferation significantly increased to 59.35% in mice treated with 90 mg/kg GLPG1205. (FIG. 30 Panel D) However, when looking at the checkpoint protein PD-1, there was no significant increase compared to MC vehicle in either the 30 mg/kg or 90 mg/kg GLPG1205 groups. (FIG. 30 Panel E)

GPR84 antagonist treatment enhances T cell differentiation after in vivo vaccination

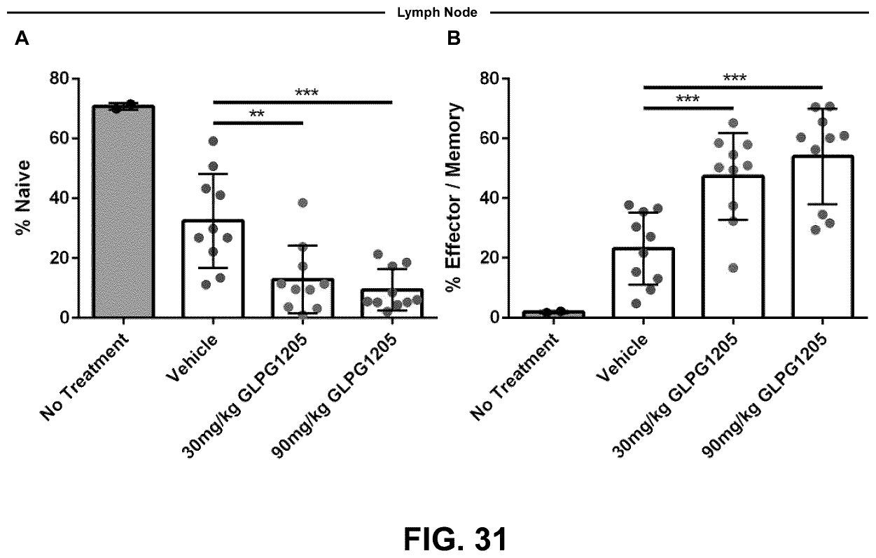

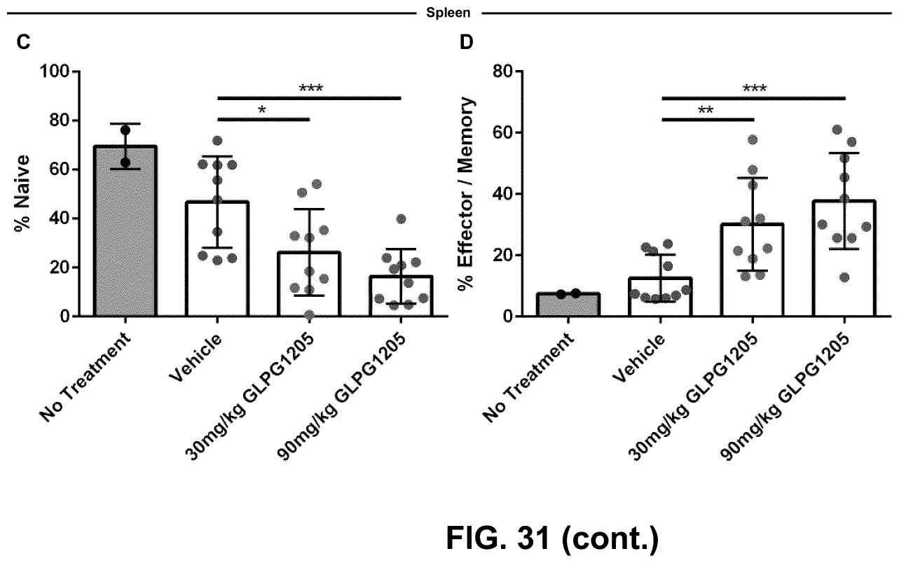

After we saw a similar proliferative effect in vivo that we previously observed in vitro, we wanted to know if we can also see similar changes in T cell differentiation. As before, we examined the naïve and effector/memory CD8 OT-I T cells in the secondary lymphoid organs. In the lymph node compartment, treatment with MC after adoptively transferred naïve OT-I T cells with sub-optimal vaccination kept 32.4% in the naïve state. This was in comparison to GLPG1205 treated mice, which exhibited significant reductions in naïve CD8 OT-I T cells at both oral doses (30 mg/kg, 12.87%; 90 mg/kg, 9.48%). (FIG. 31 Panel A) In turn, we wanted to see if these reductions translated into an increase in the effector/memory CD8 OT-I T cell phenotype. In the lymph node, mice vaccinated and treated with MC had 23.11% effector/memory CD8 T cells. As expected, we saw this compartment significantly increase to 47.38% in mice treated with 30 mg/kg GLPG1205 and further increased to 53.97% in mice treated with 90 mg/kg. (FIG. 31 Panel B) In continuity, we wanted to see if these changes were also observed in the splenic compartment. We found that vaccinated mice treated with MC maintained a 46.68% naïve CD8 OT-I T cell percentage. Similar to the lymph node compartment, we saw significant reductions in naïve CD8 OT-I T cells after oral GLPG1205 treatment. In the 30 mg/kg treatment group, we saw naïve CD8 OT-I T cells reduce to 26.16%, and were further reduced to 16.37% in the 90 mg/kg treated group. (FIG. 31 Panel C) Again, we wanted to determine if these reductions in naïve CD8 OT-I T cells correlated with significant increases in effector/memory CD8 T cells. In our control MC treated group, effector/memory CD8 OT-I T cells were 12.53%. As expected, 30 mg/kg oral GLPG1205 significantly increased this compartment to 30.12% and treatment at the higher 90 mg/kg dose further enhanced this to 37.72%. (FIG. 31 Panel D)

### GPR84 Antagonist Treatment Enhances In Vivo Anti-Tumor Therapeutic Vaccine Efficacy

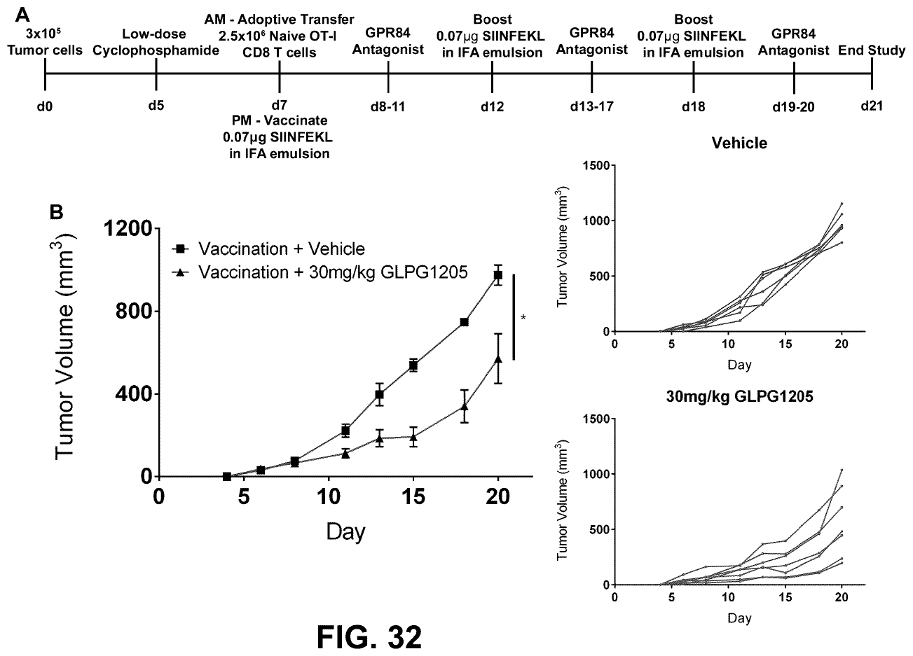

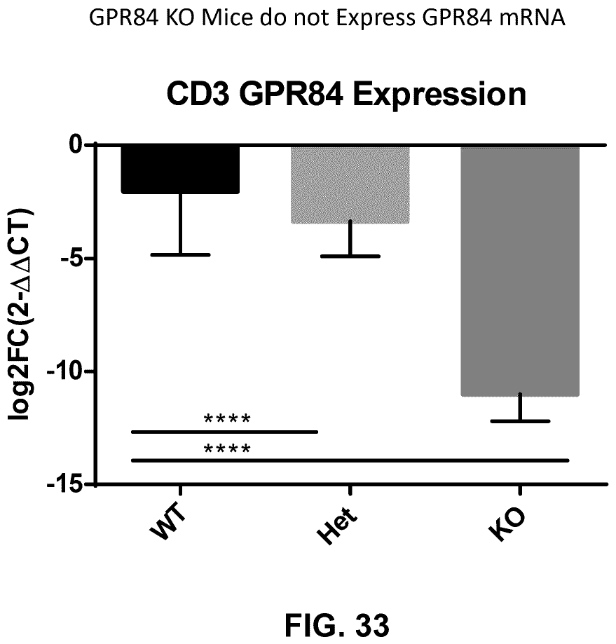

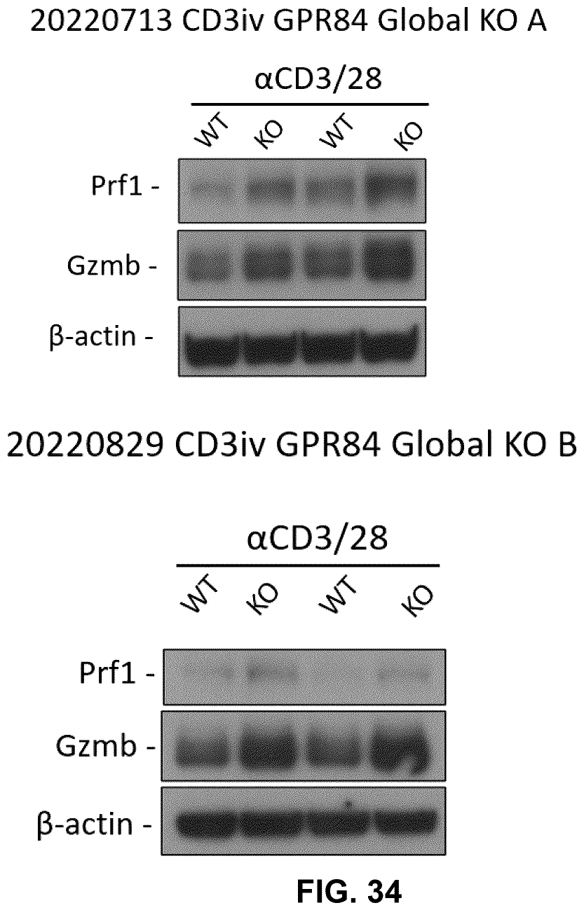

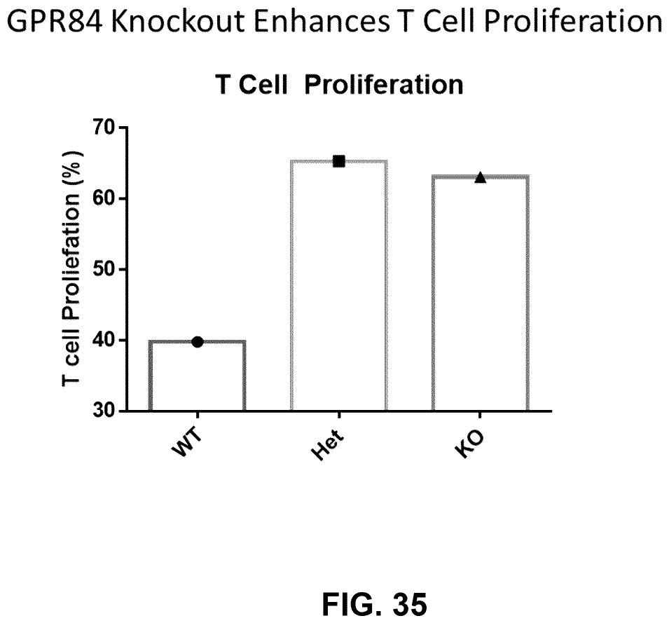

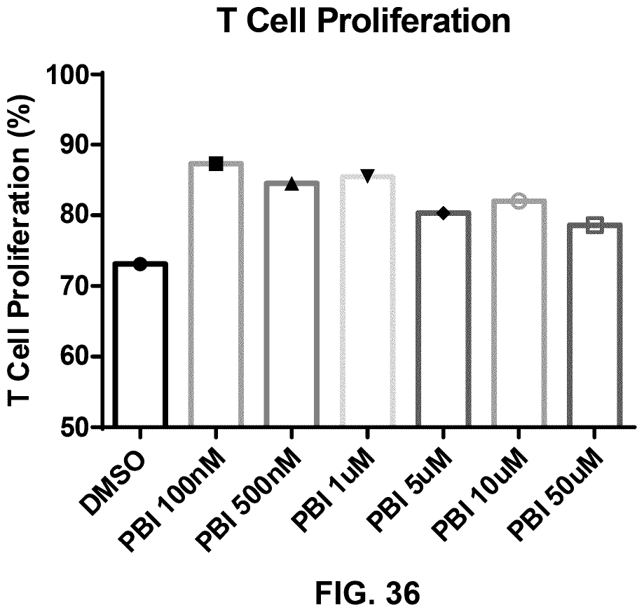

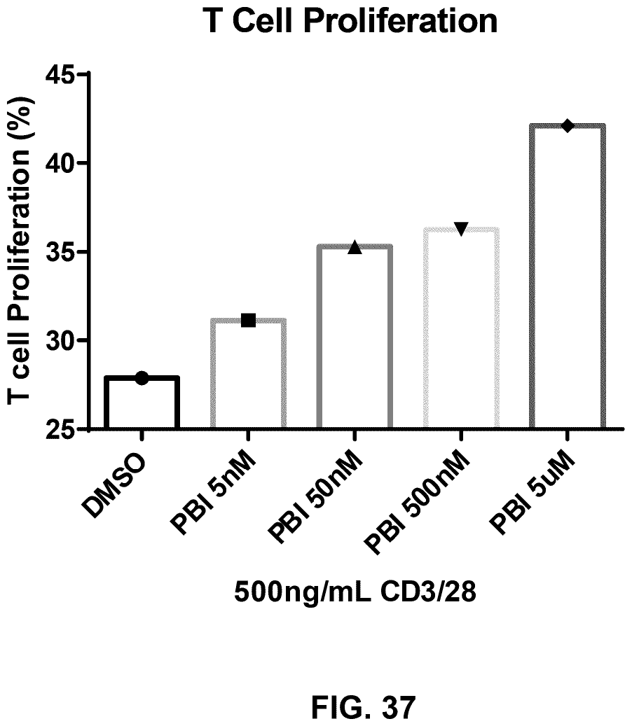

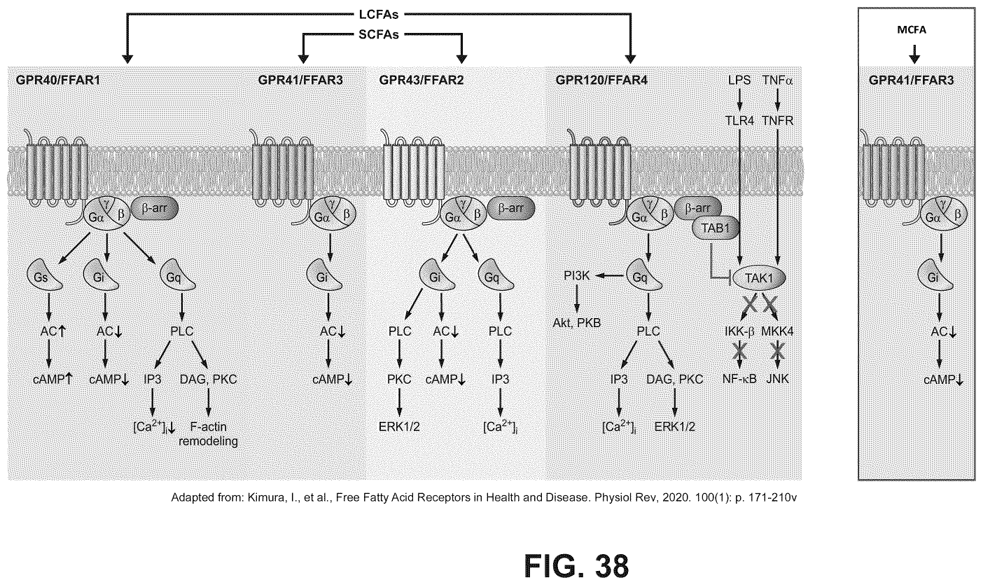

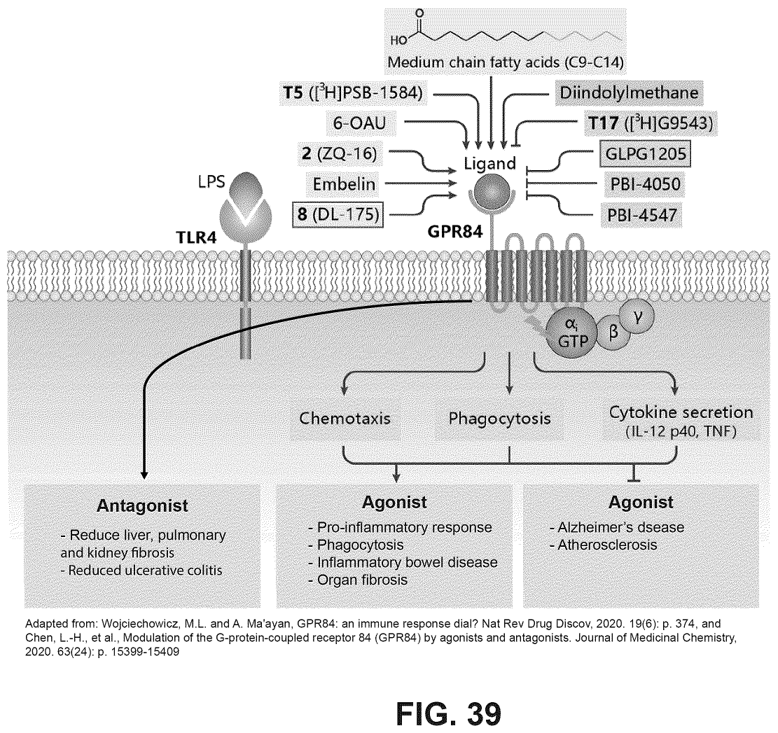

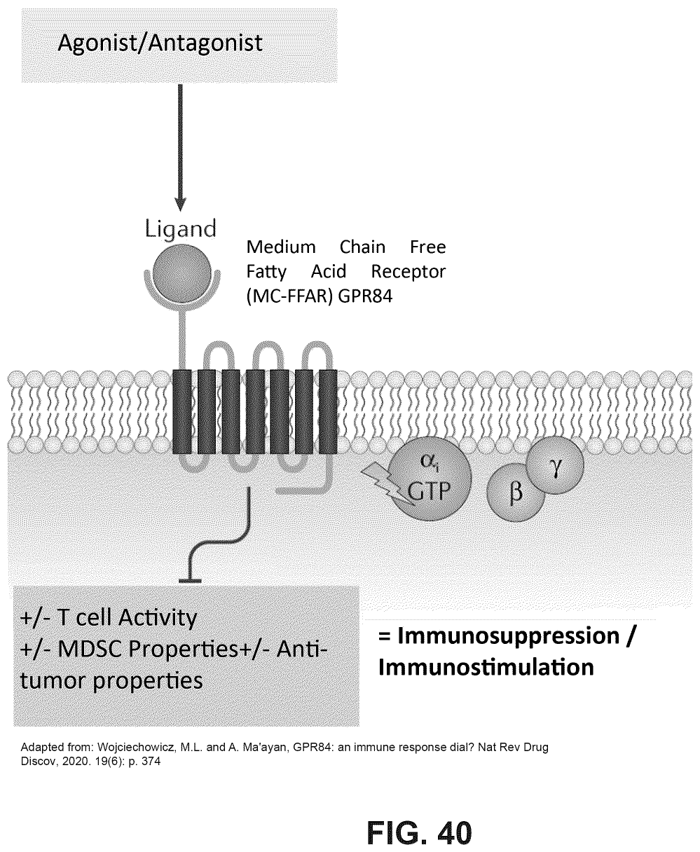

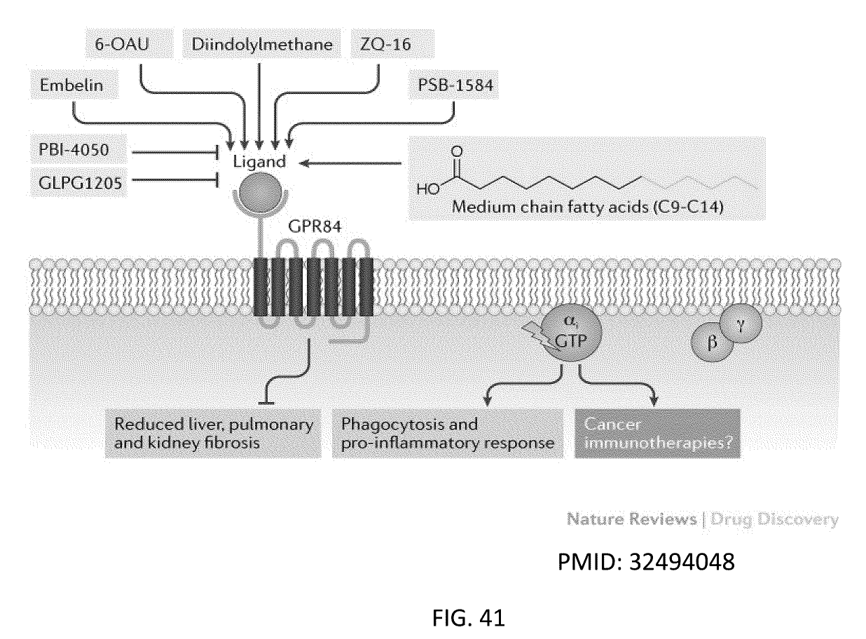

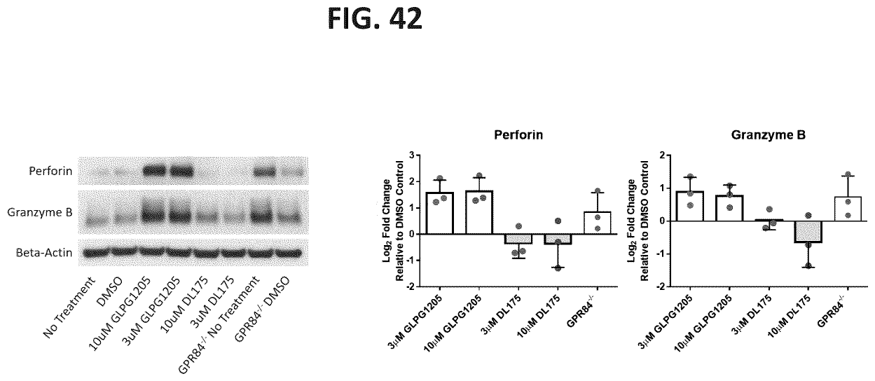

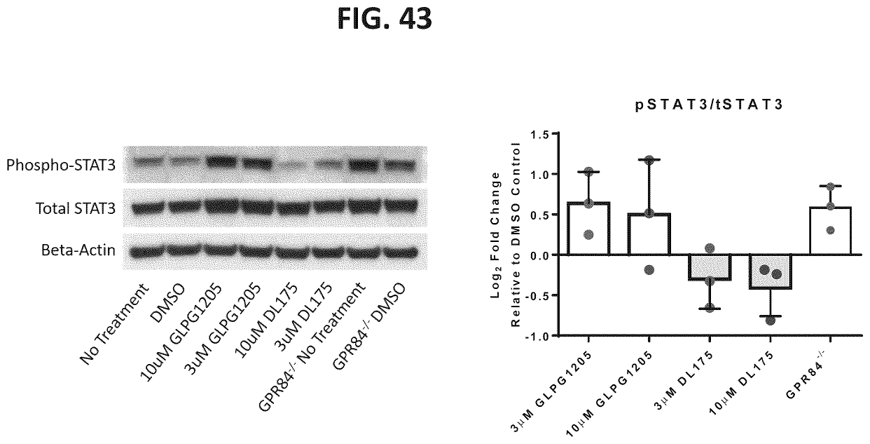

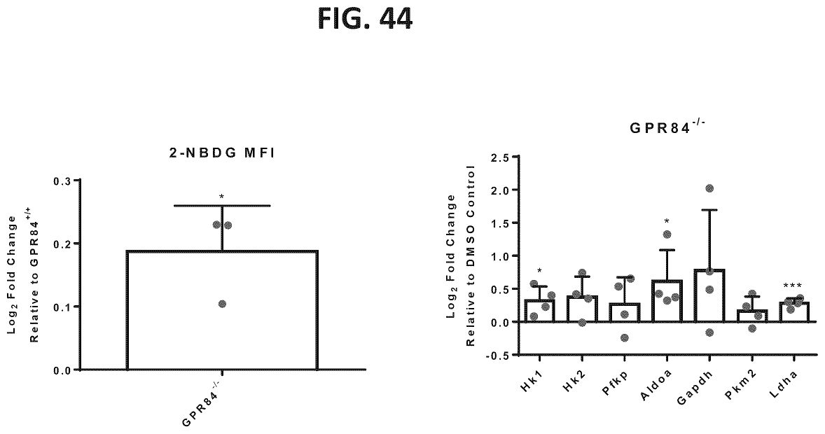

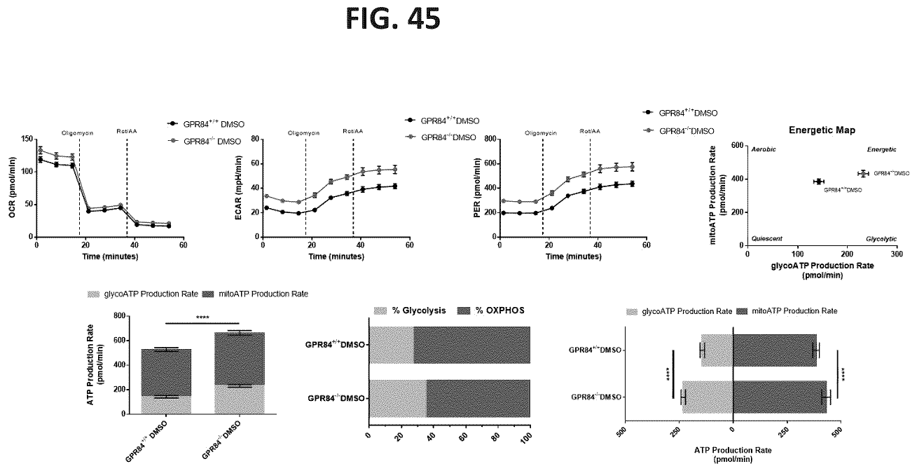

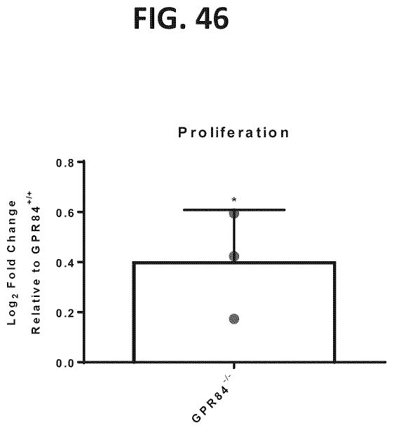

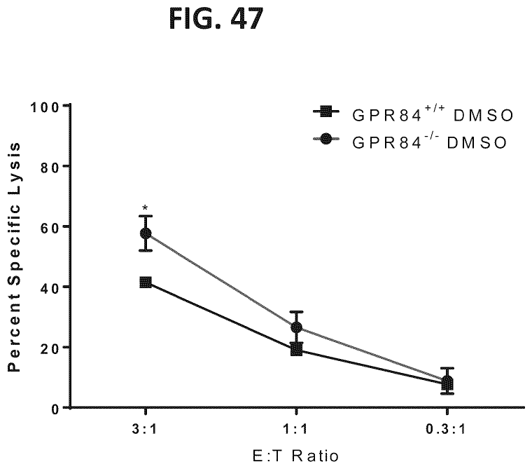

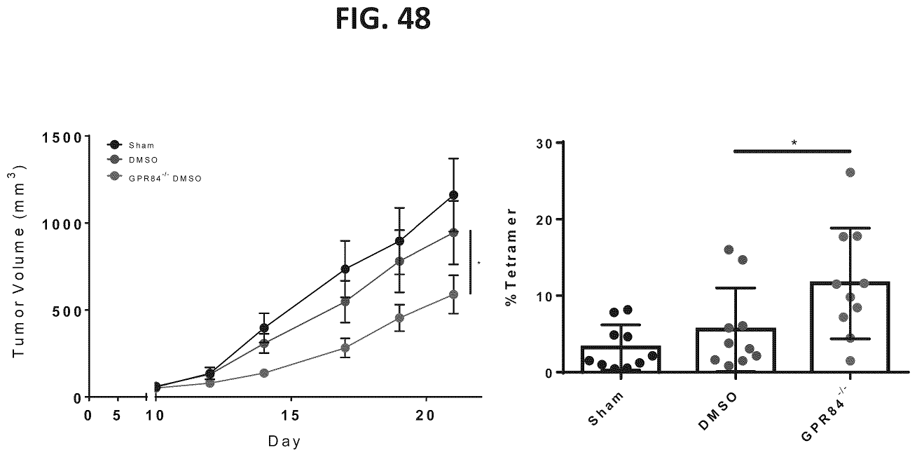

Since we demonstrated that orally administered GLPG1205 enhanced both the proliferation and differentiation of antigen specific T cells after vaccination, we wanted to see if these effects can have therapeutic efficacy. To test this, we set up a therapeutic anti-tumor vaccination model. We first injected mice with 3×10⁵ B16-OVA tumor cells into the flank of wild-type mice. Five days after tumor seeding, we injected mice with 200 g of cyclophosphamide to induce lymphodepletion. Two days after lymphodepletion we adoptively transferred 2.5×10⁶ naïve OT-I CD8 T cells and subsequently vaccinated them with 0.07 g SIINFEKL (SEQ ID NO: 1) in an IFA emulsion. We then orally administered control MC or 30 mg/kg GLPG1205 for three consecutive days. Following treatment, mice received two consecutive series of a boost containing 0.07 g SIINFEKL (SEQ ID NO: 1) in IFA emulsion and three days of treatment. (FIG. 32 Panel A) Measuring the tumor growth rates, we found that mice vaccinated and treated with 30 mg/kg GLPG1205 elicited a significantly stronger anti-tumor response compared to the MC control group. (FIG. 32 Panel B) This experiment supports our vaccination works and demonstrates that the effects on T cell expansion and differentiation after vaccination has therapeutic benefit in the treatment of cancer.

### REFERENCES CITED IN THIS EXAMPLE

1. Wang J, Wu X, Simonavicius N, Tian H, Ling L. Medium-chain fatty acids as ligands for orphan G protein-coupled receptor GPR84. J Biol Chem. Nov. 10 2006; 281(45):34457-64. doi:10.1074/jbc.M608019200
2. Yousefi S, Cooper P R, Potter S L, Mueck B, Jarai G. Cloning and expression analysis of a novel G-protein-coupled receptor selectively expressed on granulocytes. J Leukoc Biol. June 2001; 69(6):1045-52.
3. Recio C, Lucy D, Purvis GSD, et al. Activation of the Immune-Metabolic Receptor GPR84 Enhances Inflammation and Phagocytosis in Macrophages. Front Immunol. 2018; 9:1419. doi:10.3389/fimmu.2018.01419
4. Venkataraman C, Kuo F. The G-protein coupled receptor, GPR84 regulates I L-4 production by T lymphocytes in response to CD3 crosslinking. Immunology letters. November 15 2005; 101(2):144-53. doi:10.1016/j.imlet.2005.05.010
5. Zaibi M S, Kepczynska M A, Harikumar P, Alomar S Y, Trayhurn P. IL-33 stimulates expression of the GPR84 (EX33) fatty acid receptor gene and of cytokine and chemokine genes in human adipocytes. Cytokine. October 2018; 110:189-193. doi:10.1016/j.cyto.2018.05.008
6. Pillaiyar T, Kose M, Namasivayam V, et al. 6-(Ar)Alkylamino-Substituted Uracil Derivatives: Lipid Mimetics with Potent Activity at the Orphan G Protein-Coupled Receptor 84 (GPR84). ACS omega. Mar. 31, 2018; 3(3):3365-3383. doi:10.1021/acsomega.7b02092
7. Puengel T, De Vos S, Hundertmark J, et al. The Medium-Chain Fatty Acid Receptor GPR84 Mediates Myeloid Cell Infiltration Promoting Steatohepatitis and Fibrosis. Journal of clinical medicine. Apr. 16 2020; 9(4)doi:10.3390/jcm9041140
8. Gagnon L, Leduc M, Thibodeau J F, et al. A Newly Discovered Antifibrotic Pathway Regulated by Two Fatty Acid Receptors: GPR40 and GPR84. Am J Pathol. May 2018; 188(5):1132-1148. doi:10.1016/j.ajpath.2018.01.009
9. Zhang Q, Chen L H, Yang H, et al. GPR84 signaling promotes intestinal mucosal inflammation via enhancing NLRP3 inflammasome activation in macrophages. Acta Pharmacol Sin. Dec. 15, 2021; doi:10.1038/s41401-021-00825-y
10. Yin C, Cheng L, Pan J, et al. Regulatory role of Gpr84 in the switch of alveolar macrophages from CD11b(lo) to CD11b(hi) status during lung injury process. Mucosal Immunol. November 2020; 13(6):892-907. doi:10.1038/s41385-020-0321-7
11. Suzuki M, Takaishi S, Nagasaki M, et al. Medium-chain fatty acid-sensing receptor, GPR84, is a proinflammatory receptor. J Biol Chem. Apr. 12, 2013; 288(15):10684-91. doi:10.1074/jbc.M112.420042
12. Gaidarov I, Anthony T, Gatlin J, et al. Embelin and its derivatives unravel the signaling, proinflammatory and antiatherogenic properties of GPR84 receptor. Pharmacol Res. May 2018; 131:185-198. doi:10.1016/j.phrs.2018.02.021
13. Lucy D, Purvis GSD, Zeboudj L, et al. A Biased Agonist at Immunometabolic Receptor GPR84 Causes Distinct Functional Effects in Macrophages. ACS Chem Biol. Sep. 20 2019; 14(9):2055-2064. doi:10.1021/acschembio.9b00533
14. Li Y, Chung S, Li Z, et al. Fatty acid receptor modulator PBI-4050 inhibits kidney fibrosis and improves glycemic control. JCI Insight. May 17 2018; 3(10)doi:10.1172/jci.insight.120365
15. Simard J C, Thibodeau J F, Leduc M, et al. Fatty acid mimetic PBI-4547 restores metabolic homeostasis via GPR84 in mice with non-alcoholic fatty liver disease. Scientific reports. Jul. 29, 2020; 10(1):12778. doi:10.1038/s41598-020-69675-8
16. Müller M M, Lehmann R, Klassert T E, et al. Global analysis of glycoproteins identifies markers of endotoxin tolerant monocytes and GPR84 as a modulator of TNFα expression. Sci Rep. Apr. 12, 2017; 7(1):838. doi:10.1038/s41598-017-00828-y
17. Chang C H, Curtis J D, Maggi L B, Jr., et al. Posttranscriptional control of T cell effector function by aerobic glycolysis. Cell. Jun. 6, 2013; 153(6):1239-51. doi:10.1016/j.cell.2013.05.016
18. Sena L A, Li S, Jairaman A, et al. Mitochondria are required for antigen-specific T cell activation through reactive oxygen species signaling. Immunity. Feb. 21, 2013; 38(2):225-36. doi:10.1016/j.immuni.2012.10.020
19. Hossain F, Al-Khami A A, Wyczechowska D, et al. Inhibition of Fatty Acid Oxidation Modulates Immunosuppressive Functions of Myeloid-Derived Suppressor Cells and Enhances Cancer Therapies. Cancer Immunol Res. November 2015; 3(11):1236-47. doi:10.1158/2326-6066.Cir-15-003
20. Michalek R D, Gerriets V A, Jacobs S R, et al. Cutting edge: distinct glycolytic and lipid oxidative metabolic programs are essential for effector and regulatory CD4+ T cell subsets. J Immunol. Mar. 15, 2011; 186(6):3299-303. doi:10.4049/jimmunol.1003613
21. Health UDo, Services H. National stakeholder strategy for achieving health equity. US Department of Health and Human Services. 2011;
22. Foundation U H. America's Health Rankings analysis of America's Health Rankings composite measure. 2022. AmericasHealthRankings.org
23. Lauby-Secretan B, Scoccianti C, Loomis D, Grosse Y, Bianchini F, Straif K. Body Fatness and Cancer—Viewpoint of the IARC Working Group. N Engl J Med. Aug. 25 2016; 375(8):794-8. doi:10.1056/NEJMsr1606602
24. Renehan A G, Zwahlen M, Egger M. Adiposity and cancer risk: new mechanistic insights from epidemiology. Nat Rev Cancer. August 2015; 15(8):484-98. doi:10.1038/nrc3967
25. Christ A, Lauterbach M, Latz E. Western Diet and the Immune System: An Inflammatory Connection. Immunity. Nov. 19, 2019; 51(5):794-811. doi:10.1016/j.immuni.2019.09.020
26. Kimura I, Ichimura A, Ohue-Kitano R, Igarashi M. Free Fatty Acid Receptors in Health and Disease. Physiological reviews. Jan. 1, 2020; 100(1):171-210. doi:10.1152/physrev.00041.2018
27. Hara T, Kashihara D, Ichimura A, Kimura I, Tsujimoto G, Hirasawa A. Role of free fatty acid receptors in the regulation of energy metabolism. Biochimica et biophysica acta. September 2014; 1841(9):1292-300. doi:10.1016/j.bbalip.2014.06.002
28. Alvarez-Curto E, Milligan G. Metabolism meets immunity: The role of free fatty acid receptors in the immune system. Biochemical pharmacology. Aug. 15 2016; 114:3-13. doi:10.1016/j.bcp.2016.03.017
29. Hara T, Kimura I, Inoue D, Ichimura A, Hirasawa A. Free fatty acid receptors and their role in regulation of energy metabolism. Reviews of physiology, biochemistry and pharmacology. 2013; 164:77-116. doi:10.1007/112_2013_13
30. Huber A H, Kleinfeld A M. Unbound free fatty acid profiles in human plasma and the unexpected absence of unbound palmitoleate. J Lipid Res. March 2017; 58(3):578-585. doi:10.1194/jlr.M074260
31. Jump D B. Fatty acid regulation of gene transcription. Crit Rev Clin Lab Sci. 2004; 41(1):41-78. doi:10.1080/10408360490278341
32. Spector A A, Hoak J C. Letter: Fatty acids, platelets, and microcirculatory obstruction. Science. Oct. 31 1975; 190(4213):490-2. doi:10.1126/science.1166323
33. Grundmann M, Bender E, Schamberger J, Eitner F. Pharmacology of Free Fatty Acid Receptors and Their Allosteric Modulators. Int J Mol Sci. Feb. 10 2021; 22(4)doi:10.3390/ijms22041763
34. Latour M G, Alquier T, Oseid E, et al. GPR40 is necessary but not sufficient for fatty acid stimulation of insulin secretion in vivo. Diabetes. April 2007; 56(4):1087-94. doi:10.2337/db06-1532
35. Itoh Y, Kawamata Y, Harada M, et al. Free fatty acids regulate insulin secretion from pancreatic beta cells through GPR40. Nature. Mar. 13 2003; 422(6928):173-6. doi:10.1038/nature01478
36. Briscoe C P, Tadayyon M, Andrews J L, et al. The orphan G protein-coupled receptor GPR40 is activated by medium and long chain fatty acids. J Biol Chem. Mar. 28 2003; 278(13):11303-11. doi:10.1074/jbc.M211495200
37. Edfalk S, Steneberg P, Edlund H. Gpr40 is expressed in enteroendocrine cells and mediates free fatty acid stimulation of incretin secretion. Diabetes. September 2008; 57(9):2280-7. doi:10.2337/db08-0307
38. Liou A P, Lu X, Sei Y, et al. The G-protein-coupled receptor GPR40 directly mediates long-chain fatty acid-induced secretion of cholecystokinin. Gastroenterology. March 2011; 140(3):903-12. doi:10.1053/j.gastro.2010.10.012
39. Aizawa F, Nishinaka T, Yamashita T, et al. GPR40/FFAR1 deficient mice increase noradrenaline levels in the brain and exhibit abnormal behavior. J Pharmacol Sci. December 2016; 132(4):249-254. doi:10.1016/j.jphs.2016.09.007
40. Nakamoto K, Aizawa F, Miyagi K, et al. Dysfunctional GPR40/FFAR1 signaling exacerbates pain behavior in mice. PLoS One. 2017; 12(7):e0180610. doi:10.1371/joumal.pone.0180610
41. Mao X F, Wu H Y, Tang X Q, Ali U, Liu H, Wang Y X. Activation of GPR40 produces mechanical antiallodynia via the spinal glial interleukin-10/0-endorphin pathway. J Neuroinflammation. Apr. 13 2019; 16(1):84. doi:10.1186/s12974-019-1457-9
42. Mieczkowska A, Basle M F, Chappard D, Mabilleau G. Thiazolidinediones induce osteocyte apoptosis by a G protein-coupled receptor 40-dependent mechanism. J Biol Chem. Jul. 6 2012; 287(28):23517-26. doi:10.1074/jbc.M111.32481
43. Wauquier F, Philippe C, Leotoing L, et al. The free fatty acid receptor G protein-coupled receptor 40 (GPR40) protects from bone loss through inhibition of osteoclast differentiation. J Biol Chem. Mar. 1 2013; 288(9):6542-51. doi:10.1074/jbc.M112.429084
44. Tolhurst G, Heffron H, Lam Y S, et al. Short-chain fatty acids stimulate glucagon-like peptide-1 secretion via the G-protein-coupled receptor FFAR2. Diabetes. February 2012; 61(2):364-71. doi:10.2337/db11-1019
45. Tang C, Ahmed K, Gille A, et al. Loss of FFA2 and FFA3 increases insulin secretion and improves glucose tolerance in type 2 diabetes. Nat Med. February 2015; 21(2):173-7. doi:10.1038/nm.3779
46. McNelis J C, Lee Y S, Mayoral R, et al. GPR43 Potentiates 3-Cell Function in Obesity. Diabetes. September 2015; 64(9):3203-17. doi:10.2337/dbl4-1938
47. Priyadarshini M, Villa S R, Fuller M, et al. An Acetate-Specific GPCR, FFAR2, Regulates Insulin Secretion. Mol Endocrinol. July 2015; 29(7):1055-66. doi:10.1210/me.2015-1007
48. Priyadarshini M, Layden B T. FFAR3 modulates insulin secretion and global gene expression in mouse islets. Islets. 2015; 7(2):e1045182. doi:10.1080/19382014.2015.1045182
49. Veprik A, Laufer D, Weiss S, Rubins N, Walker M D. GPR41 modulates insulin secretion and gene expression in pancreatic β-cells and modifies metabolic homeostasis in fed and fasting states. Faseb j. November 2016; 30(11):3860-3869. doi:10.1096/fj.201500030R
50. Ge H, Li X, Weiszmann J, et al. Activation of G protein-coupled receptor 43 in adipocytes leads to inhibition of lipolysis and suppression of plasma free fatty acids. Endocrinology. September 2008; 149(9):4519-26. doi:10.1210/en.2008-0059
51. Horiuchi H, Kamikado K, Aoki R, et al. Bifidobacterium animalis subsp. lactis GCL2505 modulates host energy metabolism via the short-chain fatty acid receptor GPR43. Sci Rep. Mar. 5 2020; 10(1):4158. doi:10.1038/s41598-020-60984-6
52. Kimura I, Ozawa K, Inoue D, et al. The gut microbiota suppresses insulin-mediated fat accumulation via the short-chain fatty acid receptor GPR43. Nat Commun. 2013; 4:1829. doi:10.1038/ncomms2852
53. Miyamoto J, Ohue-Kitano R, Mukouyama H, et al. Ketone body receptor GPR43 regulates lipid metabolism under ketogenic conditions. Proc Natl Acad Sci USA. Nov. 19 2019; 116(47):23813-23821. doi:10.1073/pnas.191257311
54. Vinolo M A, Ferguson G J, Kulkami S, et al. SCFAs induce mouse neutrophil chemotaxis through the GPR43 receptor. PLoS One. 2011; 6(6):e21205. doi:10.1371/joumal.pone.0021205
55. Lind S, Holdfeldt A, Martensson J, et al. Functional selective ATP receptor signaling controlled by the free fatty acid receptor 2 through a novel allosteric modulation mechanism. Faseb j. June 2019; 33(6):6887-6903. doi:10.1096/fj.201802309R
56. Martensson J, Holdfeldt A, Sundqvist M, et al. Neutrophil priming that turns natural FFA2R agonists into potent activators of the superoxide generating NADPH-oxidase. JLeukoc Biol. December 2018; 104(6):1117-1132. doi:10.1002/jlb.2a0318-130rr
57. Fachi J L, Secca C, Rodrigues P B, et al. Acetate coordinates neutrophil and ILC3 responses against C. difficile through FFAR2. J Exp Med. Mar. 2 2020; 217(3)doi:10.1084/jem.20190489
58. Galvao I, Tavares L P, Correa R O, et al. The Metabolic Sensor GPR43 Receptor Plays a Role in the Control of Klebsiella pneumoniae Infection in the Lung. Front Immunol. 2018; 9:142. doi:10.3389/fimmu.2018.00142
59. Sencio V, Barthelemy A, Tavares L P, et al. Gut Dysbiosis during Influenza Contributes to Pulmonary Pneumococcal Superinfection through Altered Short-Chain Fatty Acid Production. Cell Rep. Mar. 3 2020; 30(9):2934-2947.e6. doi:10.1016/j.celrep.2020.02.013
60. Wu W, Sun M, Chen F, et al. Microbiota metabolite short-chain fatty acid acetate promotes intestinal IgA response to microbiota which is mediated by GPR43. Mucosal Immunol. July 2017; 10(4):946-956. doi:10.1038/mi.2016.114
61. Yang W, Xiao Y, Huang X, et al. Microbiota Metabolite Short-Chain Fatty Acids Facilitate Mucosal Adjuvant Activity of Cholera Toxin through GPR43. J Immunol. Jul. 1 2019; 203(1):282-292. doi:10.4049/jimmunol.1801068
62. Sina C, Gavrilova O, Forster M, et al. G protein-coupled receptor 43 is essential for neutrophil recruitment during intestinal inflammation. J Immunol. Dec. 1 2009; 183(11):7514-22. doi:10.4049/jimmunol.0900063
63. Vieira A T, Macia L, Galvao I, et al. A Role for Gut Microbiota and the Metabolite-Sensing Receptor GPR43 in a Murine Model of Gout. Arthritis Rheumatol. June 2015; 67(6):1646-56. doi:10.1002/art.39107
64. Smith P M, Howitt M R, Panikov N, et al. The microbial metabolites, short-chain fatty acids, regulate colonic Treg cell homeostasis. Science. Aug. 2 2013; 341(6145):569-73. doi: 10.1126/science.124116
65. Li Y J, Chen X, Kwan T K, et al. Dietary Fiber Protects against Diabetic Nephropathy through Short-Chain Fatty Acid-Mediated Activation of G Protein-Coupled Receptors GPR43 and GPR109A. J Am Soc Nephrol. June 2020; 31(6):1267-1281. doi:10.1681/asn.2019101029
66. Huang W, Man Y, Gao C, et al. Short-Chain Fatty Acids Ameliorate Diabetic Nephropathy via GPR43-Mediated Inhibition of Oxidative Stress and NF-κB Signaling. Oxid Med Cell Longev. 2020; 2020:4074832. doi:10.1155/2020/4074832
67. Kaye D M, Shihata W A, Jama H A, et al. Deficiency of Prebiotic Fiber and Insufficient Signaling Through Gut Metabolite-Sensing Receptors Leads to Cardiovascular Disease. Circulation. Apr. 28 2020; 141(17):1393-1403. doi:10.1161/circulationaha.119.043081
68. Maslowski K M, Vieira A T, Ng A, et al. Regulation of inflammatory responses by gut microbiota and chemoattractant receptor GPR43. Nature. Oct. 29 2009; 461(7268):1282-6. doi:10.1038/nature08530
69. Kobayashi M, Mikami D, Kimura H, et al. Short-chain fatty acids, GPR41 and GPR43 ligands, inhibit TNF-α-induced MCP-1 expression by modulating p38 and JNK signaling pathways in human renal cortical epithelial cells. Biochem Biophys Res Commun. Apr. 29 2017; 486(2):499-505. doi:10.1016/j.bbrc.2017.03.071
70. Hirasawa A, Tsumaya K, Awaji T, et al. Free fatty acids regulate gut incretin glucagon-like peptide-1 secretion through GPR120. Nat Med. January 2005; 11(1):90-4. doi:10.1038/nm1168
71. Oh D Y, Walenta E, Akiyama T E, et al. A Gpr120-selective agonist improves insulin resistance and chronic inflammation in obese mice. Nat Med. August 2014; 20(8):942-7. doi:10.1038/nm.3614
72. Gotoh C, Hong Y H, Iga T, et al. The regulation of adipogenesis through GPR120. Biochem Biophys Res Commun. Mar. 9 2007; 354(2):591-7. doi:10.1016/j.bbrc.2007.01.028
73. Gao B, Huang Q, Jie Q, et al. GPR120: A bi-potential mediator to modulate the osteogenic and adipogenic differentiation of BMMSCs. Sci Rep. Sep. 14 2015; 5:14080. doi:10.1038/srep14080
74. Lee K P, Park S J, Kang S, et al. ω-3 Polyunsaturated fatty acids accelerate airway repair by activating FFA4 in club cells. Am J Physiol Lung Cell Mol Physiol. Jun. 1 2017; 312(6):L835-1844. doi:10.1152/ajplung.00350.2016
75. Oh D Y, Talukdar S, Bae E J, et al. GPR120 is an omega-3 fatty acid receptor mediating potent anti-inflammatory and insulin-sensitizing effects. Cell. Sep. 3 2010; 142(5):687-98. doi:10.1016/j.cell.2010.07.041
76. Hudson B D, Shimpukade B, Mackenzie A E, et al. The pharmacology of TUG-891, a potent and selective agonist of the free fatty acid receptor 4 (FFA4/GPR120), demonstrates both potential opportunity and possible challenges to therapeutic agonism. Mol Pharmacol. November 2013; 84(5):710-25. doi:10.1124/mol.113.087783
77. Wittenberger T, Schaller H C, Hellebrand S. An expressed sequence tag (EST) data mining strategy succeeding in the discovery of new G-protein coupled receptors. Journal of molecular biology. Mar. 30 2001; 307(3):799-813. doi:10.1006/jmbi.2001.4520
78. Zhang Q, Yang H, Li J, Xie X. Discovery and Characterization of a Novel Small-Molecule Agonist for Medium-Chain Free Fatty Acid Receptor G Protein-Coupled Receptor 84. J Pharmacol Exp Ther. May 2016; 357(2):337-44. doi:10.1 124/jpet.116.232033
79. Peters A, Rabe P, Krumbholz P, et al. Natural biased signaling of hydroxycarboxylic acid receptor 3 and G protein-coupled receptor 84. Cell Commun Signal. Feb. 26 2020; 18(1):31. doi:10.1186/s12964-020-0516-2
80. Peters A, Rabe P, Liebing A D, et al. Hydroxycarboxylic acid receptor 3 and GPR84—Two metabolite-sensing G protein-coupled receptors with opposing functions in innate immune cells. Pharmacol Res. February 2022; 176:106047. doi:10.1016/j.phrs.2021.106047
81. Luscombe V B, Lucy D, Bataille C J R, Russell A J, Greaves D R. 20 Years an Orphan: Is GPR84 a Plausible Medium-Chain Fatty Acid-Sensing Receptor? DNA Cell Biol. November 2020; 39(11):1926-1937. doi: 10. 1089/dna.2020.5846
82. Shrestha R, Hui S-P, Imai H, et al. Plasma capric acid concentrations in healthy subjects determined by high-performance liquid chromatography. Annals of clinical biochemistry. 2015; 52(5):588-596.
83. Sills M A, Forsythe W I, Haidukewych D. Role of octanoic and decanoic acids in the control of seizures. Arch Dis Child. December 1986; 61(12):1173-7. doi:10.1136/adc.61.12.1173
84. Mohammad M A, Sunehag A L, Haymond M W. De novo synthesis of milk triglycerides in humans. Am J Physiol Endocrinol Metab. Apr. 1 2014; 306(7):E838-47. doi:10.1152/ajpendo.00605.2013
85. Sundqvist M, Christenson K, Holdfeldt A, et al. Similarities and differences between the responses induced in human phagocytes through activation of the medium chain fatty acid receptor GPR84 and the short chain fatty acid receptor FFA2R. Biochim Biophys Acta Mol Cell Res. May 2018; 1865(5):695-708. doi:10.1016/j.bbamcr.2018.02.008
86. Du Toit E, Browne L, Irving-Rodgers H, et al. Effect of GPR84 deletion on obesity and diabetes development in mice fed long chain or medium chain fatty acid rich diets. Eur JNutr. August 2018; 57(5):1737-1746. doi:10.1007/s00394-017-1456-5
87. Mahmud Z A, Jenkins L, Ulven T, et al. Three classes of ligands each bind to distinct sites on the orphan G protein-coupled receptor GPR84. Sci Rep. Dec. 20 2017; 7(1):17953. doi:10.1038/s41598-017-18159-3
88. Nagasaki H, Kondo T, Fuchigami M, et al. Inflammatory changes in adipose tissue enhance expression of GPR84, a medium-chain fatty acid receptor: TNFα enhances GPR84 expression in adipocytes. FEBS Lett. Feb. 17 2012; 586(4):368-72. doi:10.1016/j.febslet.2012.01.001
89. Saniere L, Marsais F, Jagerschmidt C, et al. Characterization of GLPG1205 in mouse fibrosis models: a potent and selective antagonist of GPR84 for treatment of idiopathic pulmonary fibrosis. A19 Less Idiopathic: Structural and Functional Abnormalities in IPF. American Thoracic Society; 2019:A1046-A1046.
90. Montgomery M K, Osborne B, Brandon A E, et al. Regulation of mitochondrial metabolism in murine skeletal muscle by the medium-chain fatty acid receptor Gpr84. Faseb j. November 2019; 33(11):12264-12276. doi:10.1096/fj.201900234R
91. Bouchard C, Page J, Bedard A, Tremblay P, Vallieres L. G protein-coupled receptor 84, a microglia-associated protein expressed in neuroinflammatory conditions. Glia. June 2007; 55(8):790-800. doi:10.1002/glia.20506
92. Huang Q, Feng D, Liu K, et al. A medium-chain fatty acid receptor Gpr84 in zebrafish: expression pattern and roles in immune regulation. Dev Comp Immunol. August 2014; 45(2):252-8. doi:10.1016/j.dci.2014.03.017
93. Kamber R A, Nishiga Y, Morton B, et al. Inter-cellular CRISPR screens reveal regulators of cancer cell phagocytosis. Nature. September 2021; 597(7877):549-554. doi:10.1038/s41586-021-03879-4
94. Wei L, Tokizane K, Konishi H, Yu H R, Kiyama H. Agonists for G-protein-coupled receptor 84 (GPR84) alter cellular morphology and motility but do not induce pro-inflammatory responses in microglia. J Neuroinflammation. Oct. 3 2017; 14(1):198. doi:10.1186/s12974-017-0970-y
95. Reyes A W B, Kim H, Huy T X N, et al. Immune-metabolic receptor GPR84 surrogate and endogenous agonists, 6-OAU and lauric acid, alter Brucella abortus 544 infection in both in vitro and in vivo systems. Microb Pathog. September 2021; 158:105079. doi:10.1016/j.micpath.2021.105079
96. Peng M, Huang B, Zhang Q, et al. Embelin inhibits pancreatic cancer progression by directly inducing cancer cell apoptosis and indirectly restricting IL-6 associated inflammatory and immune suppressive cells. Cancer Lett. Nov. 28 2014; 354(2):407-16. doi:10.1016/j.canlet.2014.08.011
97. Wu T, Dai Y, Wang W, et al. Macrophage targeting contributes to the inhibitory effects of embelin on colitis-associated cancer. Oncotarget. Apr. 12 2016; 7(15):19548-58. doi:10.18632/oncotarget.6969
98. Wu T, Wang C, Wang W, et al. Embelin impairs the accumulation and activation of MDSCs in colitis-associated tumorigenesis. Oncoimmunology. 2018; 7(11):e1498437. doi:10.1080/2162402x.2018.1498437
99. Pillaiyar T, Kose M, Sylvester K, et al. Diindolylmethane Derivatives: Potent Agonists of the Immunostimulatory Orphan G Protein-Coupled Receptor GPR84. J Med Chem. May 11 2017; 60(9):3636-3655. doi:10.1021/acs.jmedchem.6b01593
100. Nikaido Y, Koyama Y, Yoshikawa Y, Furuya T, Takeda S. Mutation analysis and molecular modeling for the investigation of ligand-binding modes of GPR84. J Biochem. May 2015; 157(5):311-20. doi:10.1093/jb/mvu075
101. Jeon E J, Davaatseren M, Hwang J T, et al. Effect of Oral Administration of 3,3′-Diindolylmethane on Dextran Sodium Sulfate-Induced Acute Colitis in Mice. J Agric Food Chem. Oct. 19 2016; 64(41):7702-7709. doi:10.1021/acs.jafc.6b02604
102. Yang S, Tan L, Chen Y, Liu A, Hong M, Peng Z. DIM mitigates the development of experimental autoimmune encephalomyelitis by maintaining the stability and suppressive function of regulatory T cells. Cell Immunol. December 2020; 358:104238. doi:10.1016/j.cellimm.2020.104238
103. Fong A M, Premont R T, Richardson R M, Yu Y R, Lefkowitz R J, Patel D D. Defective lymphocyte chemotaxis in beta-arrestin2- and GRK6-deficient mice. Proc Natl Acad Sci USA. May 28 2002; 99(11):7478-83. doi:10.1073/pnas.112198299
104. Groot-Kormelink P J, Fawcett L, Wright P D, Gosling M, Kent T C. Quantitative GPCR and ion channel transcriptomics in primary alveolar macrophages and macrophage surrogates. BMC Immunol. Oct. 26 2012; 13:57. doi:10.1186/1471-2172-13-57
105. Grouix B, Sarra-Bournet F, Leduc M, et al. PBI-4050 Reduces Stellate Cell Activation and Liver Fibrosis through Modulation of Intracellular ATP Levels and the Liver Kinase B1/AMP-Activated Protein Kinase/Mammalian Target of Rapamycin Pathway. J Pharmacol Exp Ther. October 2018; 367(1):71-81. doi:10.1124/jpet.118.250068
106. Thibodeau J F, Simard J C, Holterman C E, et al. PBI-4050 via GPR40 activation improves adenine-induced kidney injury in mice. Clin Sci (Lond). Jul. 31 2019; 133(14):1587-1602. doi:10.1042/cs20190479
107. Nguyen Q T, Nsaibia M J, Sirois M G, et al. PBI-4050 reduces pulmonary hypertension, lung fibrosis, and right ventricular dysfunction in heart failure. Cardiovasc Res. Jan. 1 2020; 116(1):171-182. doi:10.1093/cvr/cvz034
108. Khalil N, Manganas H, Ryerson C J, et al. Phase 2 clinical trial of PBI-4050 in patients with idiopathic pulmonary fibrosis. Eur Respir J. March 2019; 53(3)doi:10.1183/13993003.00663-2018
109. Labeguere F, Dupont S, Alvey L, et al. Discovery of 9-Cyclopropylethynyl-2-((S)-1-[1,4]dioxan-2-ylmethoxy)-6,7-dihydropyrimido[6,1-a]isoquinolin-4-one (GLPG1205), a Unique GPR84 Negative Allosteric Modulator Undergoing Evaluation in a Phase II Clinical Trial. J Med Chem. Nov. 25 2020; 63(22):13526-13545. doi:10.1021/acs.jmedchem.0c00272
110. Timmis H, Van Kaem T, Desrivot J, et al. GLPG1205, a GPR84 modulator: safety, pharmacokinetics, and pharmacodynamics in healthy subjects. Clinical Pharmacology in Drug Development. 2021;
111. Desrivot J, Van Kaem T, Allamassey L, Helmer E. Effect of GLPG1205, a GPR84 Modulator, on CYP2C9, CYP2C19, and CYP1A2 Enzymes: In Vitro and Phase 1 Studies. Clinical Pharmacology in Drug Development. 2021;
112. Vanhoutte F, Dupont S, Van Kaem T, et al. Human safety, pharmacokinetics and pharmacodynamics of the GPR84 antagonist GLPG1205, a potential new approach to treat IBD. J Crohns Colitisournal of Crohns & Colitis. 2015; 9(Suppl 1):5387.
113. Timmis H, Van Kaem T, Desrivot J, et al. GLPG1205, a GPR84 Modulator: Safety, Pharmacokinetics, and Pharmacodynamics in Healthy Subjects. Clin Pharmacol Drug Dev. September 2021; 10(9):994-1006. doi:10.1002/cpdd.955
114. Desrivot J, Van Kaem T, Allamassey L, Helmer E. Effect of GLPG1205, a GPR84 Modulator, on CYP2C9, CYP2C19, and CYP1A2 Enzymes: In Vitro and Phase 1 Studies. Clin Pharmacol Drug Dev. September 2021; 10(9):1007-1017. doi:10.1002/cpdd.956
115. Vermeire S, Reinisch W, Wasko-Czopnik D, et al. P610 efficacy and safety of GLPG1205, a GPR84 antagonist, in ulcerative colitis: multi-Centre proof-of-concept study. Journal of Crohn's and Colitis. 2017; 11(suppl_1):5390-5391.
116. Cottin V, Seemayer C A, Fagard L, et al. Results of a phase 2 study of GLPG1205 for idiopathic pulmonary fibrosis (PINTA). Eur Respiratory Soc; 2021.
117. Strambu I R, Fagard L, Ford P, et al. Idiopathic pulmonary fibrosis (IPF): observations from a Phase 2 trial of GLPG1205 (PINTA). Eur Respiratory Soc; 2020.
118. Dai Y, Jiao H, Teng G, et al. Embelin reduces colitis-associated tumorigenesis through limiting IL-6/STAT3 signaling. Mol Cancer Ther. May 2014; 13(5):1206-16. doi:10.1158/1535-7163.Mct-13-0378
119. Lee I S, Cho D H, Kim K S, et al. Anti-inflammatory effects of embelin in A549 cells and human asthmatic airway epithelial tissues. Immunopharmacol Immunotoxicol. February 2018; 40(1):83-90. doi:10.1080/08923973.2017.1414836
120. SreeHarsha N. Embelin impact on paraquat-induced lung injury through suppressing oxidative stress, inflammatory cascade, and MAPK/NF-κB signaling pathway. J Biochem Mol Toxicol. April 2020; 34(4):e22456. doi:10.1002/jbt.22456
121. Zhou X L, Huang L, Cao J. Embelin Reduces Systemic Inflammation and Ameliorates Organ Injuries in Septic Rats Through Downregulating STAT3 and NF-κB Pathways. Inflammation. August 2015; 38(4):1556-62. doi:10.1007/s10753-015-0130-4
122. Wu X, Liu J, Chen C, et al. 3,3′-Diindolylmethane alleviates acute atopic dermatitis by regulating T cell differentiation in a mouse model. Mol Immunol. February 2021; 130:104-112. doi:10.1016/j.molimm.2020.11.013
123. Rouse M, Singh N P, Nagarkatti P S, Nagarkatti M. Indoles mitigate the development of experimental autoimmune encephalomyelitis by induction of reciprocal differentiation of regulatory T cells and Th17 cells. Br J Pharmacol. July 2013; 169(6):1305-21. doi:10.1111/bph.12205
124. Singh N P, Singh U P, Rouse M, et al. Dietary Indoles Suppress Delayed-Type Hypersensitivity by Inducing a Switch from Proinflammatory Th17 Cells to Anti-Inflammatory Regulatory T Cells through Regulation of MicroRNA. J Immunol. Feb. 1 2016; 196(3):1108-22. doi:10.4049/jimmunol.1501727
125. Wojciechowicz M L, Ma'ayan A. GPR84: an immune response dial? Nat Rev Drug Discov. June 2020; 19(6):374. doi:10.1038/d41573-020-00029-9
126. Laurin P, Grouix B, Laverdure A, Zacharie B, Gagnon L. PBI-4050 Reduces Cardiovascular Biomarkers in Type I I Diabetic Patients With Metabolic Syndrome. Circulation. 2016; 134(suppl_1):A19469-A19469.
127. Gerriets V A, Rathmell J C. Metabolic pathways in T cell fate and function. Trends Immunol. April 2012; 33(4):168-73. doi:10.1016/j.it.2012.01.010
128. Zhang L, Romero P. Metabolic Control of CD8(+) T Cell Fate Decisions and Antitumor Immunity. Trends Mol Med. January 2018; 24(1):30-48. doi:10.1016/j.molmed.2017.11.00
129. Pearce E L, Walsh M C, Cejas P J, et al. Enhancing CD8 T-cell memory by modulating fatty acid metabolism. Nature. Jul. 2 2009; 460(7251):103-7. doi:10.1038/nature08097
130. Vitale I, Manic G, Coussens L M, Kroemer G, Galluzzi L. Macrophages and Metabolism in the Tumor Microenvironment. Cell Metab. Jul. 2 2019; 30(1):36-50. doi:10.1016/j.cmet.2019.06.001
131. Viola A, Munari F, Sanchez-Rodriguez R, Scolaro T, Castegna A. The Metabolic Signature of Macrophage Responses. Front Immunol. 2019; 10:1462. doi:10.3389/fimmu.2019.01462
132. Diskin C, Palsson-McDermott E M. Metabolic Modulation in Macrophage Effector Function. Front Immunol. 2018; 9:270. doi:10.3389/fimmu.2018.00270
133. Sukumar M, Liu J, Ji Y, et al. Inhibiting glycolytic metabolism enhances CD8+ T cell memory and antitumor function. J Clin Invest. October 2013; 123(10):4479-88. doi:10.1172/jci69589
134. Restifo N P, Dudley M E, Rosenberg S A. Adoptive immunotherapy for cancer: harnessing the T cell response. Nat Rev Immunol. Mar. 22 2012; 12(4):269-81. doi:10.1038/nri3191
135. van der Windt G J, Everts B, Chang C H, et al. Mitochondrial respiratory capacity is a critical regulator of CD8+ T cell memory development. Immunity. Jan. 27 2012; 36(1):68-78. doi:10.1016/j.immuni.2011.12.007
136. Hollingsworth R E, Jansen K. Turning the corner on therapeutic cancer vaccines. NPJ Vaccines. 2019; 4:7. doi:10.1038/s41541-019-0103-y
137. Overwijk W W. Cancer vaccines in the era of checkpoint blockade: the magic is in the adjuvant. Curr Opin Immunol. August 2017; 47:103-109. doi:10.1016/j.coi.2017.07.015
138. Kimura T, McKolanis J R, Dzubinski L A, et al. MUC1 vaccine for individuals with advanced adenoma of the colon: a cancer immunoprevention feasibility study. Cancer Prev Res (Phila). January 2013; 6(1):18-26. doi:10.1158/1940-6207.Capr-12-027
139. Finn O J. The dawn of vaccines for cancer prevention. Nat Rev Immunol. March 2018; 18(3):183-194. doi:10.1038/nri.2017.140

### Example 7

Described herein is the use of an agonist to the medium chain free fatty acid receptor (FFAR) GPR84 to modify the immune response in both T cells and myeloid cells, to provide clinical benefit such that it treats an existing disease and improves the survival and outcomes. The simultaneous immune modulation of T cell and myeloid cell function is surprising and as shown herein can have multiple therapeutic applications.

For example, the use of an agonist to GPR84 FFAR can simultaneously affect myeloid and T cells by increasing the immunosuppressive functions of myeloid cells (monocytes, macrophages, granulocytes—including eosinophils, and myeloid-derived suppressor cells) and decreasing the cytotoxic activity and cytokine production of T cells. The decrease in T cell function is represented by a decrease in the expression of perform or granzyme B in CD8 T cells and a decreased cytokine production inf CD4T cells. The decrease in immunosuppressive functions in myeloid cells is shown by a decrease in arginase 1 and iNOS. This type of immune modulation can be beneficial in diseases with inflammation (chronic or acute) including colitis, asthma, glomerulonephritis, hepatitis, myocarditis, transplant rejection and others where the immune system is attacking one's organs. In a similar way one can treat patients in a pre-diabetic stage when the immune response is attacking the beta-cells of the pancreas and eventually leads to the development of full-blown diabetes. The opposite effect occurs when using an antagonist to GPR84 FFAR. Immune cells show an increase the cytotoxic (increased in granzyme B and perform) in CD8 T cells and in helper function (cytokine production) in the CD4 T cells. The antagonist also causes a decrease the immunosuppressive function of myeloid cells by diminishing the expression of arginase 1 and iNOS. This type of immune modulation can be used to increase the response to a vaccine, to increase the response to an infectious agent such as COVID-19 or influenza, to improve the efficacy of an anti-cancer treatment, be it chemotherapy, radiation therapy or immunotherapy.

The combination of an agonist and an antagonist of the GPR84 FFAR can be used in the generation of T cells for immunotherapy purposes, such as CAR-T cells. For example, it has been shown that keeping CAR-T cells in a state of “stemness”. i.e. not fully mature and active, increases their survival and their therapeutic function when transferred in vivo into mice with tumors or in cancer patients. Without wishing to be bound by theory, the use of an agonist during the in vitro generation and culture of CAR-T cells can keep them in a state of “stemness”, while still being able to divide and increase in number. Once the CAR-T cells are harvested and infused into a patient with cancer, then one can treat the patient with an antagonist which can increase the maturation and cytotoxic function of the CAR-T cells as they reach the tumor. In addition, the antagonist can decrease the immunosuppressive function of the myeloid cells in the tumor, allowing the CAR-T cells to fully execute their anti-tumor effect.

There are a variety of energy sources that immune cells use to carry out their function. Two drivers of cellular energy are derived from fats and sugars. Fats which contain fatty acids and are used in fatty acid oxidation, while sugars are the substrate used in glycolysis. Fatty acids are made of various carbon lengths and degrees of saturation. These variations can change the way and where fatty acids are used in tissue. Currently, there are four families of fatty acids. These are short-chain fatty acids (<5 carbons), medium-chain fatty acids (MCFA) (6-12 carbons), long-chain fatty acids (13-21 carbons) and very-long-chain fatty acids (>22 carbons). Short-chain fatty acids are synthesized by gut intestinal fermentation of indigestible foods. Medium, long, and very-long chains can be synthesized from cells or obtained through the diet. Recently, FFAs have been more extensively studied as multiple reports have found that they play an important role in regulating metabolic disorders.

This regulatory role does not solely rely on fatty acids as a component of cellular energy, but also through their use as a signaling ligands. Free fatty acids (FFA) bind to a family of G-protein coupled receptors (GPCRs), entitled free fatty acid receptors (FFAR). FFARs are found on various cells in the body and ligation with these GPCRs commits an intracellular signaling cascade which can have global effects on the phenotype, physiology, and action of the cell. Without wishing to be bound by theory, the presence of FFARs can act as an energy sensor and contribute towards regulation of cellular metabolism. FFARs structures restrict the length of the FFA that can bind to them. Currently, there are five main FFARs. These are FFAR1, FFAR2, FFAR3, FFAR4, and GPR84. FFAR1 and FFAR4 only bind long-chain FFA. FFAR2 and FFAR3 can bind to short-chain FFA. The fifth, and least explored FFAR, GPR84, has greatest affinity for medium-chain FFAs. Herein, we will validate the role that the medium chain FFAR GPR84 has on the immune system.

GPR84 is a medium-chain fatty acid receptor. It was found through a data mining strategy searching for GPCRs. Still defined as an orphan receptor, its ligand, MCFAs, was not discovered until years later. Further examination found that GPR84 is Gi/o GPCR, and interaction with its ligand leads to an intracellular reduction of cyclic adenosine monophosphate (cAMP). GPR84 is distributed among various tissues in the body, such as adipocytes, but also, GPR84 is found to have high expression in immune cells. In studies of adipogenesis, GPR84 was upregulated in human adipocytes stimulated with inflammatory cytokines and mice fed with high-fat diet.

In the immune tissue, GPR84 is expressed in monocytes, macrophages, granulocytes, T cells, and B cells. These studies found that immune cells exposed to a pro-inflammatory stimulus induce high expression of GPR84. Other metabolically contributing factors which increase GPR84 expression in macrophages include hyperglycemia, hypercholesterolemia, and oxidized LDL. Studies examining the role of GPR84 in T cells found that GPR84 deficient mice increased IL-4, IL-5 and IL-13, but had no effect on IL-2 or IFN-gamma production.

Without wishing to be bound by theory, these studies indicate that GPR84 acted as a feed-forward mechanism during inflammation. In this mechanism, a pro-inflammatory stimulus increases the expression of GPR84, and further signaling through GPR84 exacerbates this inflammatory response. This pathway was shown in one study where immune cells exposed to LPS induced GPR84 and that supplementing with MCFA increased the production of IL-12p40. These feed-forward pro-inflammatory findings developed a therapeutic void and lead to the development of GPR84 agonists. The synthetic agonist, 6-OAU, was found the increase chemotaxis of human polymorphonuclear leukocytes and macrophages and increase proinflammatory cytokine production. In a separate study, GPR84 agonism under inflammatory conditions elevated pro-inflammatory cytokines and increased bacterial phagocytosis and this elevated response was abrogated in GPR84 deficient mice or in macrophages treated with a GPR84 antagonist. A separate natural GPR84 agonist, Embelin, was also examined. Here, studies found that treatment with Embelin lead to neutrophil chemotaxis and primed these cells for oxidative burst and that loss of GPR84 abrogated the pro-inflammatory cytokine secretion. Also, a synthesized GPR84 agonist, DL175, was also found to promote phagocytosis of in human myeloid cells. In addition, there have been studies which have examined the effect of GPR84 agonists in cancer cells. However, most were conducted by examining the direct effect of agonist on cancerous cell lines in vitro. The sole agonist used in vivo was conducted in nude mice, which lack the immune system and cannot provide any evidence that GPR84 agonists modulate the immune system. These controlled systems have raised skepticism that in these studies the agonists used cannot be acting through the GPR84 receptor at all.

These findings regarding myeloid chemotaxis and metabolic regulation lead researchers to examine GPR84's anti-inflammatory and metabolic regulation. In studies using antagonists, a goal was to prevent chemotaxis and to elicit and anti-inflammatory response. The first antagonist created, PBI-4050, was found to be both an agonist of GPR40 and an antagonist of GPR84. PHI-4050 was found to be both an agonist of GPR40 and an antagonist of GPR84. PBI-4050 was found to affect macrophages, fibroblasts, myofibroblasts, and epithelial cells. Antagonism reduced fibrotic capacity of these cells while also dampening macrophage activation and secretion of pro-inflammatory and pro-fibrotic molecules. In addition, macrophage recruitment was prevented, known contributors to fibrotic disease. In a similar approach, the use of GLPG1205, a GPR84 specific receptor antagonist was used in the reduction of inflammation and fibrosis. This study also found that antagonism of GPR84 led reductions in neutrophils, monocytes, and macrophage migration. When examined in three separate models of NASH, there was a significant decrease in macrophage accumulation as well as reductions in both inflammation and fibrosis. Both studies showed similar outcomes, and both indicated that the use of antagonisms as an anti-inflammatory and anti-fibrotic drug can be therapeutic.

Another study examined the role of GPR84 in metabolic deregulation. Supplementing previous GPR84 antagonist literature, this study examined PBI-4547, a GPR40 and PPAR gamma agonist with some GPR84 antagonist, was studied in Non-alcoholic fatty liver disease (NAFLD). A model of diet-induced obesity was used and antagonism of GPR84 with PBI-4547 improved metabolic dysregulation through reductions in hyperglycemia and hypertriglyceridemia. Interestingly, when examining the effects in GPR84 deficient mice, this ability to regulate the metabolism was absent suggesting GPR84 receptor can play a major role. The mechanistic ability to regulate metabolism was due to an ability to modulate glycolysis and fatty acid oxidation. Researchers found increases in glucose transporters and upregulations of enzymes which leading to improved glycolysis. In addition, increases in fatty acid oxidation led to improve metabolism of fatty acid oxidation. Both metabolic adjustments reduced overall metabolic dysregulation and returned the system back to physiological homeostasis. A separate study found that the use of a separate GPR84 modulator, PBI-4050, also improved glycemic control, suggesting that GPR84 can have an important role in metabolic regulation.

Our data indicates that by using an antagonist or an agonist of GPR84 FFAR one can manipulate the inflammatory response such that it can cause a therapeutic effect in diseases associated with inflammation. Inflammation can refer to the effect caused by T lymphocytes and myeloid cells (macrophages and myeloid-derived suppressor cells). For example, one can want to increase (strengthen) the protective response of the immune system in an infectious disease such as COVID-19 or in cancer by increasing the ability of T cells to kill cells infected by the virus or tumor cells. Data described herein indicate that the use of an antagonist to GPR84 FFAR causes an increase in the proteins granzyme Band perform which are used by T cells to kill infected cells or tumor cells. This happens while simultaneously inhibiting the myeloid cells that decrease the function of T cells. Furthermore, in a disease such as COVID-19, one of the mechanisms that causes severe pulmonary complications, is the invasion of the lungs by myeloid-derived suppressor cells producing high levels of arginase 1. Data described herein indicate that the use of a GPR84 FFAR antagonist decreases the expression and production of arginase 1 and decreases its ability to block T cell function.

When using a GPR84 FFAR agonist, stimulation of immune cells with a GPR84 agonist causes a decrease in T cell cytotoxic mechanisms and increases the immunosuppressive mechanisms (arginase 1 and nitric oxide synthase) in MDSC. This leads to a decrease in T cell mediated inflammation by the increased function of MDSC. This approach can be useful in diseases mediated by inflammation such as autoimmune diseases including colitis, rheumatoid arthritis, lupus, Crohn's disease and others. Furthermore, it can be used to prevent diseases where the immune system causes damage to important structures including, but not limited to, diabetes mellitus (diabetes Type I), myocarditis, and the rejection of transplanted organs including solid organs (kidneys, liver, lung, heart) or bone marrow transplants.

Modulating immune cells in inflammatory diseases can play a pivotal role in managing disease progression. The value of the GPR84 receptor is its duality of function. We found that the use of a GPR84 agonist exhibits an anti-inflammatory immune profile, while a GPR84 antagonist was found to be pro-inflammatory. This contradicts current literature which suggests that GPR84 agonists are pro-inflammatory and antagonists are anti-inflammatory. The benefit of this duality is that in diseases in which the immune system is over activated, such as ulcerative colitis, Crohn's disease, Rheumatoid arthritis, Lupus, having the ability to modulate the immune system towards an anti-inflammatory state with a GPR84 agonist can be therapeutic. In contrast, the use of a GPR84 antagonist in diseases where the immune response is underactive, like cancer, can be beneficial

Immunomodulatory treatments to block inflammation can be used more often in medicine than those that increase the inflammatory response. Treatments to block inflammation can make use of drugs or antibodies that block the production of inflammatory cytokines or block the binding of these cytokines to their receptors. Examples include monoclonal antibodies directed against TNF (infliximab, adalimumab, golimumab and certolizumab), inhibitors of ILS (etanercept), inhibitors of IL4/IL13 (Dupilumab). There are the use of potent overall inhibitors including corticosteroids such as prednisone, triamcinolone and many others, or even chemotherapeutic agents. However, the use of modulators that can simultaneously modulate the function of T cells and myeloid cells to achieve a therapeutic response have not been reported.

The use of FFARs in metabolism has been studied. The modulation of immune cells through FFARs can provide opportunities to modulate inflammatory diseases. While most of the therapies have been studied in the more well studied FFARI-4, pharmacologic approaches have been made against the MCFA receptor GPR84. Literature has indicated that the use GPR84 agonists contributes to a pro-inflammatory response, while antagonists are suggested to be anti-inflammatory. Our alternative mechanism is in which the agonism of the GPR84 receptor leads to a dampening and anti-inflammatory response, while antagonism leads to a proinflammatory response. Our mechanism is different because it describes the use of GPR84 opposite of what has been reported. This surprising ability to modulate the function of inflammatory cells can have broad implications in the treatment of inflammatory diseases. In the case of diseases associated with inflammation such as ulcerative colitis, the use of a GPR84 agonist to promote an anti-inflammatory response can be beneficial. On the other side, diseases or circumstances where inflammation is desired, such as boosting the immune response after vaccination with weak antigens, the use of a GPR84 antagonist can be beneficial. Such an ability to modulate the activity of the immune system can be an integral component in inflammatory based diseases.

There are no known GPR84 agonists which have successfully made it to clinical trials. There are explanations for this which and can require further research before clinical trials and an approved therapy is provided. DIM, which is debated as a known allosteric modulator versus a direct agonist has been studied in vivo. However, these studies have been conducted in nude mice which lack an immune system, and therefore its direct role on immune tissue is unknown. Additional studies have examined this phenomenon, and even with synthesized agonists of GPR84, off-target effects were still found, muddying the ability to conclude that therapeutic effects were directly due to agonism of the GPR84 receptor. More research can be required to identify and examine a stable direct GPR84 agonist pre-clinically in vivo before movement into clinical trials can be warranted. A study which examined how quickly a GPR84 specific agonists (DL-175) was metabolized found that when exposed to mouse hepatocytes, this pharmacological agent was rapidly degraded. Such a finding can pose limitations as direct specific agonists of GPR84 can be structurally prone to rapid metabolism and clearance and can prevent their use for in vivo/clinical studies.

Various GPR84 antagonists have been brought to clinical trials. PBI-4050 and GLPG1205, the two known GPR84 antagonists have both been tested in phase II clinical trials and have strong safety profiles. In regard to the safety profile, PBI-4050 is used as a treatment for idiopathic pulmonary fibrosis. Preliminary results found that the GPR84 antagonist was well tolerated both as a single agent and in combination with nintedanib or pirfenidone in a phase II clinical trial. Results found that the ability to increase forced vital capacity was unsuccessful in patients both as a single agent and in combination with nintedanib. More so, without wishing to be bound by theory, combination therapy of PBl-4050 with pirfenidone worsened forced vital capacity, which can indicate acceleration of disease. Arguments for a reduction in FVC and acceleration in disease can be due to a drug-drug interaction between PBI-4050 and pirfenidone. When further examining the effects of PBI-4050 on inflammatory and fibrotic biomarkers, results found that patients treated with PBI-4050 had increase in IL-9, IL-7, MIP-1beta, with increases in IL-1Ra, IL-13, and G-CSF. While the argument of reduction of inflammation occurs, some of these markers such as MIP-1beta and G-CSF are common markers of inflammation. Such inflammatory increases can support the position whereby a currently unknown pro-inflammatory pathway can be stimulated with GPR84 antagonism.

PBI-4050 has also been examined as a treatment in type II diabetic patients with metabolic syndrome. A phase II study found that treatment with PBl-4050 can reduce HbAlc in type II diabetic patients with metabolic syndrome. Such results can pose to be promising in our proposal of modulation the immune system, as immune cell activity is able to be manipulated by metabolic regulation.

The other GPR84 antagonist that has been used in clinical trials is GLPG1205. GLPG1205 is a negative allosteric modulator of GPR84 that has progressed through phase II clinical trials. Initial phase I studies found that GLPG1205 occupied the human GPR84 receptor, was tolerated and had a terminal half-life of 30.1 to 140 hours. Additionally, when studying the interaction of GLPG1205 with the cytochrome family of enzymes and were found to have strong safety profiles indicating that this drugs administration can be used concomitantly with other therapies.

GLPG1205 has also been studied in the treatment of interstitial pulmonary fibrosis. Results provided a non-significant reduction in fibrosis in all groups that were supplemented with GLPG1205 over the standard of care, which warranted further studies to determine if GLPG1205 does have an effect in this disease.

GLPG1205 use was also explored as a treatment for ulcerative colitis. Results from this study found that GLPG1205 was unable to improve the inflammatory response. In fact, GLPG1205 was found to lead to worsening of colitis in certain patients in the treatment study which led to the drug's discontinuation. Without wishing to be bound by theory, the antagonism of the GPR84 receptor can lead to a pro-inflammatory response.

We do not foresee limitations with the use of GPR84 antagonists for implementation. Both drugs studied in the clinic have been touted to influence the immune system and inflammation. In addition, GPR84 antagonists have been used in diseases of metabolic dysregulation which supports that these drugs can be used in diseases of inflammation and can be excellent candidates as immune modulators in inflammatory diseases.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method of treating cancer, preventing cancer, or the recurrence of cancer in a subject, the method comprising administering to the subject a therapeutically effective amount of a GPR84 antagonist.

2. (canceled)

3. (canceled)

4. A method of modulating the immune system of a subject afflicted with or at risk of cancer, the method comprising administering to the subject a therapeutically effective amount of a GPR84 antagonist.

5. The method of claim 4, wherein modulating comprises increasing the cytotoxicity of T-cells, decreasing immunosuppressive effects of myeloid-derived-suppressor cells, or both.

6. The method of claim 1, wherein the GPR84 antagonist comprises a compound according to:

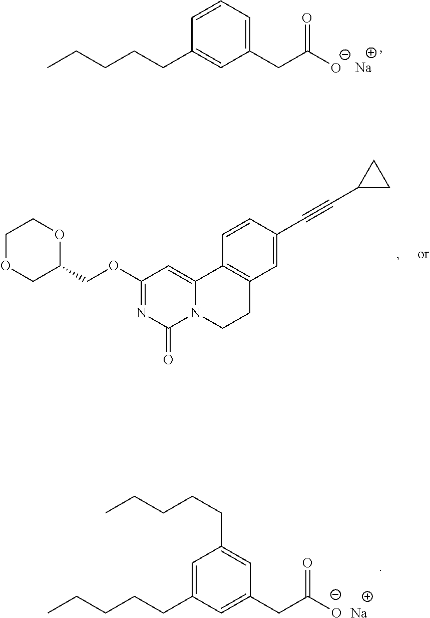 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

7. The method of claim 1, wherein the therapeutically effective amount comprises about 10 mg/kg to about 90 mg/kg.

8. The method of claim 1, wherein the GPR84 antagonist is administered daily, weekly, biweekly, or monthly.

9. The method of claim 1, wherein the method further comprises administering to the subject at least one additional anti-cancer agent.

10. The method of claim 9, wherein the at least one additional anti-cancer agent comprises an immunotherapeutic agent, a chemotherapeutic agent, radiation therapy, or any combination thereof.

11. The method of claim 10, wherein the immunotherapeutic agent comprises an immune checkpoint inhibitor, an adoptive T-cell therapy, a monoclonal antibody, an oncolytic virus therapy, a cancer vaccine, an immune system modulator, or any combination thereof.

12. The method of claim 11, wherein the monoclonal antibody comprises an immune checkpoint inhibitor.

13. The method of claim 11, wherein the monoclonal antibody comprises anti-OX-40, anti-PD-1, anti-PD-L1, anti-LAG3, or anti-CTLA-4.

14. The method of claim 13, wherein the anti-PD-1 antibody comprises a pembrolizumab, nivolumab, or cemiplimab.

15. The method of claim 13, wherein the anti-CTLA-4 antibody comprises Ipilimumab.

16. The method of claim 11, wherein the adoptive T-cell therapy comprises tumor-infiltrating lymphocyte (TIL) therapy, engineered T-cell receptor (TCR) therapy, CAR T-cell therapy, or natural killer (NK) cell therapy.

17. The method of claim 10, wherein the at least one chemotherapeutic agent comprises cyclophosphamide, gemcitabine, 5-fluorouracil, docetaxel, doxorubicin, oxaliplatin, mitoxantrone, melphalan, or anthracyclines.

18. The method of claim 1, wherein the cancer comprises breast cancer, prostate cancer, colorectal cancer, cervical cancer, lung cancer, lymphoma, leukemia, pancreatic cancer, liver cancer, brain cancer, or skin cancer.

19. The method of claim 1, wherein the subject has a familial history of cancer, a chronic inflammatory condition, or a genomic mutation.

20. The method of claim 19, wherein the inflammatory condition comprises colitis, chronic prostatitis.

21. The method of claim 19, wherein the genomic mutation comprises a mutation of BRCA1, BRCA2, MLH1, MSH2, MSH6, PMS2, EPCAM, APC, or MUTYH.

22. A method of activating a T cell, the method comprising contacting a T cell with a GPR84 antagonist, wherein the GPR84 antagonist activates the T cell.

23. The method of claim 22, further comprising the step of activating the T cell with an additional stimuli.

24. The method of claim 23, wherein the stimuli comprises anti-CD3, anti-CD28, PSA, Muc-1, MAGE, carcinoembryonic antigen (CEA) or a combination thereof.

25. The method of claim 22, wherein the method is an ex vivo method.

26. The method of claim 22, wherein the GPR84 antagonist comprises a compound according to:

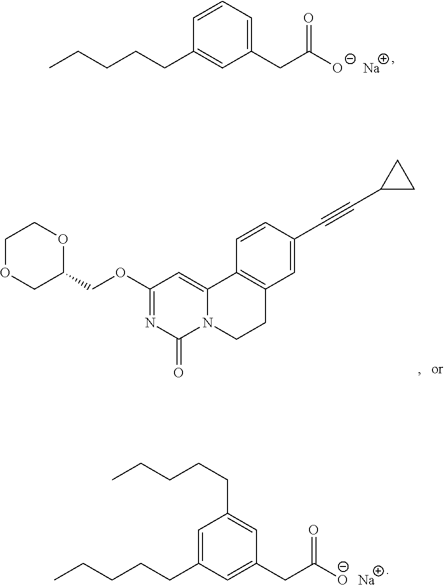 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

27. The method of claim 22, wherein the T cell comprises a CAR T cell.

28. The method of claim 22, further comprising the steps of obtaining a T cell from a subject, and culturing the T cell in a medium comprising a GPR84 antagonist.

29. The method of claim 22, further comprising the step of administering to a subject a therapeutically effective amount of the GPR84 antagonist.

30. The method of claim 22, wherein the T cell is dormant prior to culturing.

31. The method of claim 22, further comprising the step of administering the activated T cell to a subject.

32. A pharmaceutical composition comprising a GPR84 antagonist, at least one additional anti-cancer agent, and a pharmaceutically acceptable carrier, excipient, or diluent.

33. The pharmaceutical composition of claim 32, wherein the GPR84 antagonist comprises a compound according to:

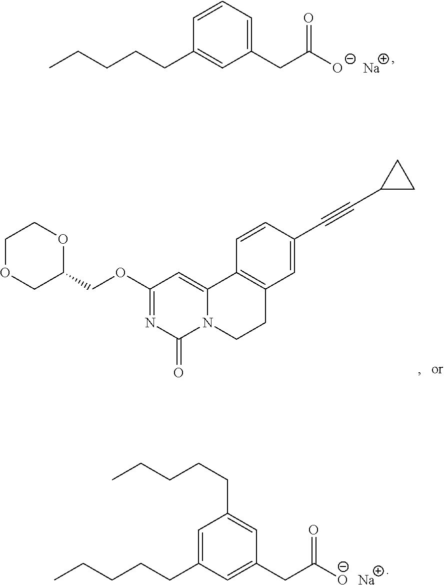 [SMILES: CCCCCc1cccc(CC(=O)[O-])c1 ; [Na+] ; O=c1nc(OC[C@@H]2COCCO2)cc2n1CCc1cc(C#CC3CC3)ccc1-2 ; CCCCCc1cc(CCCCC)cc(CC(=O)[O-])c1 ; [Na+]]

34. The pharmaceutical composition of claim 32, wherein the at least one additional anti-cancer agent comprises an immunotherapeutic agent or a chemotherapeutic agent.

35. The pharmaceutical composition of claim 34, wherein the immunotherapeutic agent comprises a CAR T-cell, an anti-cancer antibody, or both.

36. The pharmaceutical composition of claim 35, wherein the anti-cancer antibody comprises an anti-checkpoint inhibitor antibody.

37. The pharmaceutical composition of claim 34, wherein the at least one chemotherapeutic agent comprises cyclophosphamide, gemcitabine, 5-fluorouracil, docetaxel, doxorubicin, oxaliplatin, mitoxantrone, melphalan, or anthracyclines.
